(19) United States
(12) Patent Application Publication
(10) Pub. No.: US 2013/0178437 A1
(43) Pub. Date: Jul. 11, 2013

(54) CONJUGATES COMPRISING HYDROXYALKYL STARCH AND A CYTOTOXIC AGENT AND PROCESS FOR THEIR PREPARATION

(71) Applicants: Helmut Knoller, Friedberg (DE); Dominik Heckmann, Friedberg (DE); Frank Hacket, Altenstadt (DE); Norbert Zander, Bad Nauheim (DE)

(72) Inventors: Helmut Knoller, Friedberg (DE); Dominik Heckmann, Friedberg (DE); Frank Hacket, Altenstadt (DE); Norbert Zander, Bad Nauheim (DE)

(73) Assignee: FRESENIUS KABI DEUTSCHLAND GMBH, Bad Homburg (DE)

(21) Appl. No.: 13/809,069

(22) PCT Filed: Jul. 11, 2011
(86) PCT No.: PCT/EP2011/003460

§ 371 Date: Mar. 28, 2013

Related U.S. Application Data
(60) Provisional application No. 61/362,832, filed on Jul. 9, 2010.

(30) Foreign Application Priority Data
Jul. 9, 2010 (EP) 10007094.5

(51) Int. Cl. C07H 15/24

(52) U.S. Cl. CPC C07H 15/24
USPC 514/34; 536/16.8

(57) ABSTRACT

The present invention relates to hydroxyalkyl starch (HAS) conjugates and to a method for preparing the hydroxyalkyl starch (HAS) conjugates, the hydroxyalkyl starch (HAS) conjugates comprising a hydroxyalkyl starch derivative and a cytotoxic agent, said conjugate having a structure according the following formula HAS′(-M)ₙ wherein M is a residue of a cytotoxic agent, the cytotoxic agent comprising a carbonyl group, HAS′ is a residue of the hydroxyalkyl starch derivative comprising at least one functional group X, n is greater than or equal to 1, and wherein the cytotoxic agent is linked via the carbonyl function present in the cytotoxic agent to the functional group X comprised in the hydroxyalkyl starch derivative, wherein the linkage via the carbonyl function is a cleavable linkage, which is capable of being cleaved in vivo so as to release the cytotoxic agent.

The present invention relates to hydroxyalkyl starch (HAS) conjugates comprising a hydroxyalkyl starch derivative and a cytotoxic agent, said conjugate having a structure according the following formula

HAS′(-M)ₙ

wherein M is a residue of a cytotoxic agent, the cytotoxic agent comprising a carbonyl group,
HAS′ is a residue of the hydroxyalkyl starch derivative comprising at least one functional group X, n is greater than or equal to 1, preferably in the range of from 3 to 200, preferably in the range of from 3 to 100, and wherein the cytotoxic agent is linked via the carbonyl function present in the cytotoxic agent to the functional group X comprised in the hydroxyalkyl starch derivative, wherein the linkage via the carbonyl function is a cleavable linkage, which is capable of being cleaved in vivo so as to release the cytotoxic agent. Moreover, besides the conjugate, the invention relates to the method for preparing said conjugate and conjugates obtained or obtainable by said method. Further, the invention relates to the HAS cytotoxic agent conjugates for the treatment of cancer as well as to pharmaceutical compositions comprising these conjugates for the treatment of cancer.

Hydroxyalkyl starch (HAS), in particular hydroxyethyl starch (HES), is a substituted derivative of the naturally occurring carbohydrate polymer amylopectin, which is present in corn starch at a concentration of up to 95% by weight, and is degraded by other amylases in the body. HES in particular exhibits advantageous biological properties and is used as a blood volume replacement agent and in hemodilution therapy in clinics (Sommermeyer et al., 1987, Krankenhauspharmazie, 8(8): 271-278; Weidler et al., 1991, Arzneimittelforschung/Drug Research, 41: 494-498).

Cytotoxic agents are natural or synthetic substances which decrease the cell growth. A major drawback of many cytotoxic agents is their extreme low water solubility which renders the in vivo administration of the agent extremely complicated. Thus, this poor water solubility usually has to be overcome by complex formulation techniques including various excipients, wherein these excipients usually also show toxic side effects.

As an example, the emulsifier Cremophor EL and ethanol, which are used to formulate taxol-based agents in order to deliver the required dosis of these taxol-based agents in vivo, shows toxic effects such as vasodilation, dispnea, and hypotension. In particular, Cremophor EL has also been shown to cause severe anaphylactic hypersensitivity reactions, hyperlipidaemia, abnormal lipoprotein patterns, aggregation of erythrocytes and peripheral neuropathy (“Cremophor EL: the drawbacks and advantages of vehicle selection for drug formulation”, European Journal of Cancer”, Volume 31, Issue 13, Pages 1590-1598). In fact, the maximum dose of, for example paclitaxel, a taxol-based cytotoxic agent that can be administered to mice by injection, is dictated by the acute lethal toxicity of said Cremophor EL vehicle. Further, the low water solubility of antracyclines at neutral pH requires acidic formulations which may cause problems during intravenous injection.

This is one reason why the potential use of soluble prodrugs, in particular macromolecular prodrugs, as a means of administering biologically effective cytotoxic agents to mammals has been proposed. Such prodrugs include chemical derivatives of the cytotoxic agents which, upon administration, will eventually liberate the active parent compound in vivo.

Besides the low water solubility of many cytotoxic agents, cytotoxic agents as such often show high non-specific toxicity, i.e. toxicity which is not directed against the specific target, in particular the tumor, said non-specific toxicity having a limiting effect on the maximum dose which can be administered. Anthracyclines, for example, are known to show high unspecific cardiotoxicity.

Prodrugs have been proposed to provide an advantageous targeting and/or an enhancement of the stability of the therapeutic agent. Further, such prodrugs were suggested to prolong the circulation lifetime, to provide an extended duration of activity, or to achieve a reduction of side effects and drug toxicity.

Thus, the preparation of prodrugs of cytotoxic agents is of high interest in order to enhance the water solubility and/or modify the onset and/or duration of action of the cytotoxic agent in vivo while preferably minimizing any unspecific toxicity.

A typical example in the preparation of prodrugs of cytotoxic agents involves the conversion of alcohols or thioalcohols to either organic phosphates or esters (Remington's Pharmaceutical Science, 16^{th} ed., A. Ozols (ed.), 1980).

Numerous reviews have described the potential application of macromolecules as high molecular weight carriers for cytotoxic agents yielding in polymeric prodrugs of said agents. It was proposed that by coupling the cytotoxic agents to polymers, it is possible to increase the molecular weight and size of the prodrug so that the weight and size of the prodrugs are too high to be quickly removed by glomerular filtration in the kidney and that, as consequence, the plasma residence time can be drastically increased.

Most modifications to date have been carried out with polyethylene glycol, HPMA (N-(2-hydroxypropyl)methacrylamide) or similar polymers with polyethylene glycol (PEG) being generally preferred as polymer because of its easy availability and the possibility to give defined products upon reaction of limited available functional groups for coupling to a cytotoxic agent being present in PEG.

For example, WO 93/24476 discloses conjugates between taxane-based drugs, such as paclitaxel, to polyethylene glycol as macromolecule. In these conjugates, paclitaxel is linked to the polyethylene glycol using an ester linkage.

Similarly, U.S. Pat. No. 5,977,163 describes the conjugation of taxane-based drugs, such as paclitaxel or docetaxel, to similar water soluble polymers such as polyglutamic acid or polyaspartic acid.

Likewise, polyethylene glycol conjugates with cytotoxic agents, such as camptothecins, are disclosed in WO 98/07713. According to WO 98/07713, the polymer is linked via a linker to a hydroxyl function of the cytotoxic agent providing an ester linkage which allows for a rapid hydrolysis of the polymer drug linkage in vivo to generate the parent drug. This is achieved by using a linker comprising an electron-withdrawing group in close proximity to the ester bond. No polysaccharide-based conjugates were disclosed in WO 98/07713.

In a similar way, the influence of sterically demanding groups on the release rate of cytotoxic agents being incorporated into polyethylene glycol conjugates has been described in WO 01/146291A1.

U.S. Pat. No. 6,395,266 B1 discloses branched PEG polymers linked to various cytotoxic agents. The branched polymers are considered to be advantageous compared to linear PEG conjugates since a higher loading of parent drug per unit of polymer can be achieved. The actual activity of these conjugates in vivo for the treatment of cancer was, however, not shown.

Similar to U.S. Pat. No. 6,395,266 B 1, EP 1 496 076 A1 discloses Y-shaped branched hydrophilic polymer derivatives conjugated to cytotoxic agents such as camptothecin. Again, the actual activity of these conjugates in vivo was not shown.

In a similar way, the following patent and non-patent literature discloses PEG conjugates: Greenwald et al., J. Med. Chem., 1996, 39: 424-431 and U.S. Pat. No. 5,840,900.

Further, PEG doxorubicin conjugates were described in Rodrigues et al., Bioorganic Medicinial Chemistry 7 (1999) 2517-2524. In these conjugates, the doxorubicin is coupled to the polymer via hydrazide linkages. In a similar way, Rodrigues et al. described the coupling of daunorubicin via hydrazide linkages to PEG (Rodrigues et al., Bioorganic Medicinal Chemistry 14 (2006) 4110-4117).

PEG, however, is known to have unpleasant or hazardous side effects such as induction of antibodies against PEG (N. J. Ganson, S. J. Kelly et al., Arthritis Research & Therapie 2006, 8:R12) and nephrotoxicity (G. A. Laine, S. M. Hamid Hossain et al., The Annals of Pharmacotherapy, 1995 November, Volume 29) on use of such PEG or PEG-related conjugates. In addition, the biological activity of the active ingredients is most often greatly reduced in some cases after the PEG coupling. Moreover, the metabolism of the degradation products of PEG conjugates is still substantially unknown and possibly represents a health risk. Further, the functional groups available for coupling to cytotoxic agents are limited, so a high loading of the polymer with the respective drug is not possible.

Thus, there is still a need for physiologically well tolerated alternatives to such PEG conjugates with which the residence time of low molecular weight substances in the plasma can be increased and/or the efficacy of these drugs can be increased and/or non-specific toxicity can be decreased. Further, there is the need for macromolecular prodrugs which provide an advantageous targeting of the tumor and/or which, upon administration, will eventually liberate the active parent compound in vivo with improved pharmacodynamic properties.

It would be particularly desirable to provide prodrugs which take advantage of the so-called Enhanced Permeability and Retention (EPR) effect. This EPR effect describes the property by which certain sizes of molecules, such as macromolecules or liposomes, tend to accumulate in tumor tissue much more than they do in normal tissue (reference is made to respective passages of U.S. Pat. No. 6,624,142 B2; or to Vasey P. A., Kaye S. B., Morrison R., et al. (January 1999) “Phase I clinical and pharmacokinetic study of PK1 [N-(2-hydroxypropyl)methacrylamide copolymer doxorubicin]: first member of a new class of chemotherapeutic agents-drug-polymer conjugates. Cancer Research Campaign Phase I/II Committee”. Clinical Cancer Research 5 (1): 83-94). The general explanation for that effect is that tumor vessels are usually abnormal in form and architecture. This is due to the fact that, in order for tumor cells to grow quickly, they must stimulate the production of blood vessels.

Without wanting to be bound to any hypothesis, it is contemplated that the EPR effect allows for an enhanced or even substantially selective delivery of macromolecules to the tumor cells and as consequence, enrichment of the macromolecules in the tumor cells, when compared to the delivery of these molecules to normal tissue.

WO 03/074088 describes hydroxyalkyl starch conjugates with, for example, cytotoxic agents such as daunorubicin, wherein the cytotoxic agent is usually directly coupled via an amino group to the hydroxyalkyl starch yielding in 1:1 conjugates. No use of these conjugates in vivo was shown. Further, in WO 03/074088 no cleavable linkage between the cytotoxic agent and hydroxyalkyl starch was described, which, upon administration, would be suitable to readily liberate the active drug in vivo.

Thus, there is still the need to provide new prodrugs of cytotoxic agents being bound to advantageous polymers for the treatment of cancer in vivo.

Thus, it is an object of the present invention to provide novel conjugates comprising a polymer linked to a cytotoxic agent. Further, it is an object of the present invention to provide a method for preparing such conjugates. Additionally, it is an object of the present invention to provide pharmaceutical compositions comprising these novel conjugates as well as the use of the conjugates and the pharmaceutical composition, respectively, in the treatment of cancer.

Surprisingly, it was found that linking of a cytotoxic agent via a cleavable linkage to hydroxyalkyl starch derivatives may lead to conjugates showing at least one of the desired beneficial properties, such as improved drug solubility, and/or optimized drug residence time in vivo, and/or reduced toxicity, and/or high efficiency, and/or effective targeting of tumor tissue in vivo.

Without wanting to be bound to any theory, it is believed that the specific biodegradable hydroxyalkyl starch polymers exhibit a preferred chemical constitution and as a result prevent the elimination of the intact conjugate—comprised of the polymer and the cytotoxic agent—through the kidney prior to any release of the cytotoxic agent. Thus, rapid elimination of the cytotoxic agent through the kidney by filtration through pores may be avoided. Preferably, the specific biodegradable hydroxyalkyl starch polymers of the invention comprised in the conjugate additionally exhibit an optimized mean molecular weight MW and/or an optimized molar substitution MS, together with the above mentioned preferred overall chemical constitution, so as to allow for a degradability of the hydroxyalkyl starch polymer comprised in the conjugate and release of the cytotoxic agent in a favorable time range. Further, it is believed that in contrast to most of the polymers described in the prior art, such as polyethylene glycol and derivatives thereof, the polymer fragments obtained from degradation of the conjugate of the present invention can be removed from the bloodstream by the kidneys or degraded via the lysosomal pathway without leaving any unknown degradation products of the polymer in the body.

Without wanting to be bound to any theory as to how the conjugates of the invention might operate, it is further believed that at least some of the conjugates of the invention might be able to deliver the respective cytotoxic agent into extracellular tissue space, such as into tissue exhibiting an EPR effect. However, it has to be understood that it is not intended to limit the scope of the invention only to such conjugates which take advantage of the EPR effect; also conjugates which show, possibly additionally, different advantageous characteristics, such as advantageous activity and/or low toxicity in vivo due to alternative mechanisms, are encompassed by the present invention.

Thus, the present invention relates to a hydroxyalkyl starch (HAS) conjugate comprising a hydroxyalkyl starch derivative and a cytotoxic agent, said conjugate having a structure according to the following formula

HAS′(-M)ₙ

wherein M is a residue of a cytotoxic agent, the cytotoxic agent comprising a carbonyl group,
HAS′ is a residue of the hydroxyalkyl starch derivative comprising at least one functional group X, and n is greater than or equal to 1, preferably in the range of from 3 to 200, more preferably in the range of from 3 to 100, and wherein the cytotoxic agent is linked via the carbonyl function present in the cytotoxic agent to the functional group X comprised in the hydroxyalkyl starch derivative, wherein the linkage via the carbonyl function is a cleavable linkage, which is capable of being cleaved in vivo so as to release the cytotoxic agent, and wherein HAS′ preferably has a mean molecular weight MW above the renal threshold.

The term “linked via the carbonyl function” is denoted to mean, that the hydroxyalkyl starch is reacted with the carbonyl function of the cytotoxic agent, thereby forming a linkage between the residue of the hydroxyalkyl starch derivative and the carbonyl C atom of M.

The structure HAS′(-M)ₙ as used in the context of the present invention encompasses embodiments in which the residue of the cytotoxic agent is linked via a single bond to the hydroxyalkyl starch derivative as well as embodiments in which the residue of the cytotoxic agent M is linked via a double bond to the residue of the hydroxyalkyl starch derivate. In case the cytotoxic agent is linked via a double bond to the hydroxyalkyl starch derivative, the structure may also be written in the following way: HAS′(=M)ₙ.

Preferably, the hydroxyalkyl starch derivative is linked via a double bond to the former carbonyl C atom of the cytotoxic agent, said double bond being formed upon reaction of the functional group X with the carbonyl group of the cytotoxic agent.

In experiments conducted by Waitzinger et al. (Clin. Drug Invest. 1998; 16: 151-160) and reviewed by Jungheinrich et al. (Clin Pharmacokinet. 2006; 44(7): 681-699), the renal threshold for hydroxyethyl starch was determined to be between 45 and 60 kD.

Further, the present invention relates to a method for preparing a hydroxyalkyl starch (HAS) conjugate comprising a hydroxyalkyl starch derivative and a cytotoxic agent, said conjugate having a structure according to the following formula

HAS′(-M)ₙ

wherein M is a residue of a cytotoxic agent, wherein the cytotoxic agent comprises a carbonyl group,
HAS′ is a residue of the hydroxyalkyl starch derivative comprising at least one functional group X, n is greater than or equal to 1, preferably in the range of from 3 to 200, preferably in the range of from 3 to 100, and wherein the cytotoxic agent is linked via the carbonyl function present in the cytotoxic agent to a functional group X comprised in the hydroxyalkyl starch derivative, wherein the linkage via the carbonyl function is a cleavable linkage, which is capable of being cleaved in vivo so as to release the cytotoxic-agent, said method comprising
(a) providing a hydroxyalkyl starch (HAS) derivative, said HAS derivative comprising a functional group Z¹; and providing a cytotoxic agent comprising a carbonyl group;
(b) coupling the HAS derivative to the cytotoxic agent, wherein the functional group Z¹ comprised in the hydroxyalkyl starch derivative is coupled directly to the carbonyl group of the cytotoxic agent thereby forming the functional group —X—.

Moreover, the present invention relates to a hydroxyalkyl starch conjugate obtainable or obtained by the above-mentioned method. Further, the present invention relates to a pharmaceutical compound or composition comprising the hydroxyalkyl starch conjugate or the hydroxyalkyl starch conjugate obtainable or obtained by the above-mentioned method. In addition, the present invention relates to the hydroxyalkyl starch conjugate as described above, or the pharmaceutical composition as described above, for the use as a medicament, in particular for the treatment of cancer. Moreover, the present invention relates to the use of the hydroxyalkyl starch conjugate as described above, or the pharmaceutical composition as described above for the manufacture of a medicament for the treatment of cancer. Moreover, the present invention relates to a method of treating a patient suffering from cancer comprising administering a therapeutically effective amount of the hydroxyalkyl starch conjugate as described above, or the pharmaceutical composition as described above.

### The Hydroxyalkyl Starch

In the context of the present invention, the term “hydroxyalkyl starch” (HAS) refers to a starch derivative having a constitution according to the following formula (III)

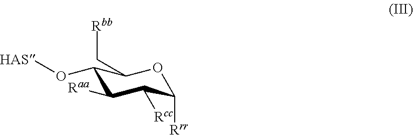

wherein the explicitly shown ring structure is either a terminal or a non-terminal saccharide unit of the HAS molecule and wherein HAS″ is a remainder, i.e. a residual portion of the hydroxyalkyl starch molecule, said residual portion forming, together with the explicitly shown ring structure containing the residues R^{aa}, R^{bb} and R^{cc} and R^{rr} the overall HAS molecule. In formula (III), R^{aa}, R^{bb} and R^{cc} are independently of each other —O-HAS″, hydroxyl or a linear or branched hydroxyalkyl group, in particular the group —O-HAS″ or —[O—(CR^{w}R^{x})—(CR^{y}R^{z})]ₓ—OH wherein R^{w}, R^{x}, R^{y} and R^{z} are independently of each other selected from the group consisting of hydrogen and alkyl, x is an integer in the range of from 0 to 20, preferably in the range of from 0 to 4. Preferably, R^{aa}, R^{bb} and R^{cc} are independently of each other selected from the group consisting of —[O—CH₂—CH₂], —OH with s being in the range of from 0 to 4, and the group —O-HAS″. In particular, R^{aa}, R^{bb} and R^{cc} are independently of each other —OH, O—CH₂—CH₂—OH (2-hydroxyethyl), and —O-HAS″. Residue R^{rr} is —O-HAS″ in case the explicitly shown ring structure is a non-terminal saccharide unit of the HAS molecule. In case the explicitly shown ring structure is a terminal saccharide unit of the HAS molecule, R^{rr} is —OH, and formula (III) shows this terminal saccharide unit in its hemiacetal form. This hemiacetal form, depending on e.g. the solvent, may be in equilibrium with the free aldehyde form as shown in the scheme below:

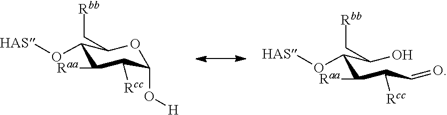

The term —O-HAS″ as used in the context of the residue R^{rr} as described above is, in addition to the remainder HAS″ shown at the left hand side of formula (III), a further remainder of the HAS molecule which is linked as residue R^{rr} to the explicitly shown ring structure of formula (III)

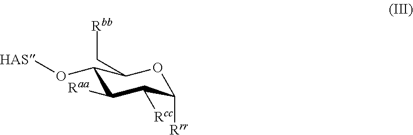

and forms, together with the residue HAS″ shown at the left hand side of formula (III) and the explicitly shown ring structure the overall HAS molecule.

Each remainder HAS″ discussed above comprises, preferably essentially consists of—apart from terminal saccharide units—one or more repeating units according to formula (IIIA)

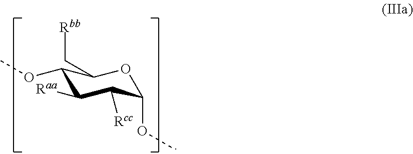

According to the present invention, the HAS molecule shown in formula (III) is either linear or comprises at least one branching point, depending on whether at least one of the residues R^{aa}, R^{bb} and R^{cc} of a given saccharide unit comprises yet a further remainder —O-HAS″. If none of the R^{aa}, R^{bb} and R^{cc} of a given saccharide unit comprises yet a further remainder —O-HAS″, apart from the HAS″ shown at the left hand side of formula (III), and optionally apart from HAS″ contained in R^{rr}, the HAS molecule is linear.

Hydroxyalkyl starch comprising two or more different hydroxyalkyl groups is also conceivable. The at least one hydroxyalkyl group comprised in the hydroxyalkyl starch may contain one or more, in particular two or more, hydroxyl groups. According to a preferred embodiment, the at least one hydroxyalkyl group contains only one hydroxyl group.

The term “hydroxyalkyl starch” as used in the context of the present invention also includes starch derivatives wherein the alkyl group is suitably mono- or polysubstituted. Such suitable substituents are preferably halogen, especially fluorine, and/or an aryl group. Yet further, instead of alkyl groups, HAS may comprise also linear or branched substituted or unsubstituted alkenyl groups.

Hydroxyalkyl starch may be an ether derivative of starch, as described above. However, besides of said ether derivatives, also other starch derivatives are comprised by the present invention, for example derivatives which comprise esterified hydroxyl groups. These derivatives may be, for example, derivatives of unsubstituted mono- or dicarboxylic acids with preferably 2 to 12 carbon atoms or of substituted derivatives thereof. Especially useful are derivatives of unsubstituted monocarboxylic acids with 2 to 6 carbon atoms, especially derivatives of acetic acid. In this context, acetyl starch, butyryl starch and propynyl starch are preferred.

Furthermore, derivatives of unsubstituted dicarboxylic acids with 2 to 6 carbon atoms are preferred. In the case of derivatives of dicarboxylic acids, it is useful that the second carboxy group of the dicarboxylic acid is also esterified. Furthermore, derivatives of monoalkyl esters of dicarboxylic acids are also suitable in the context of the present invention. For the substituted mono- or dicarboxylic acids, the substitute group may be preferably the same as mentioned above for substituted alkyl residues. Techniques for the esterification of starch are known in the art (cf. for example Klemm, D. et al., Comprehensive Cellulose Chemistry, vol. 2, 1998, Wiley VCH, Weinheim, N.Y., especially Chapter 4.4, Esterification of Cellulose (ISBN 3-527-29489-9)).

According to a preferred embodiment of the present invention, a hydroxyalkyl starch (HAS) according to the above-mentioned formula (III)

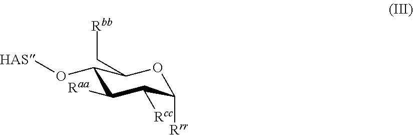

is employed. The saccharide units comprised in HAS″, apart from terminal saccharide units, may be the same or different, and preferably have the structure according to the formula (IIIa)

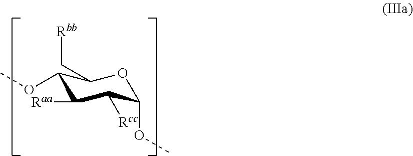

as shown above.

According to the invention, the term “hydroxyalkyl starch” is preferably a hydroxyethyl starch, hydroxypropyl starch or hydroxybutyl starch, wherein hydroxyethyl starch is particularly preferred.

Thus, according to the present invention, the hydroxyalkyl starch (HAS) is preferably a hydroxyethyl starch (HES), the hydroxyethyl starch preferably having a structure according to the following formula (III)

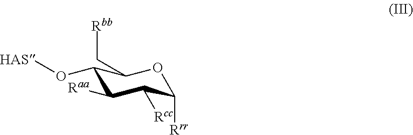

wherein R^{aa}, R^{bb} and R^{cc} are independently of each other selected from the group consisting of —O-HES″, and —[O—CH₂—CH₂]ₛ—OH, wherein s is in the range of from 0 to 4 and wherein HAS″ is, in case the hydroxyalkyl starch is hydroxyethyl starch, the remainder of the hydroxyethyl starch and could be abbreviated with HES″. Residue R^{rr} is either —O-HAS″ (which, in case the hydroxyalkyl starch is hydroxyethyl starch, could be abbreviated with —O-HES″) or, in case the formula (III) shows the terminal saccharide unit of HES, R^{rr} is —OH. For the sake of consistency, the abbreviation “HAS” is used throughout all formulas in the context of the present invention, and if HAS is concretized as HES, it is explicitly mentioned in the corresponding portion of the text.

### The Term “Hydroxyalkyl Starch Derivative”

In the context of the present invention, the term “hydroxyalkyl starch derivative” refers to a derivative of starch being functionalized with at least one functional group Z¹, said group being a functional group capable of being linked to (reacted with) a further compound, in particular to the carbonyl group of the cytotoxic agent, which in turn is comprised in above-defined conjugate having a structure according to the following formula

HAS′(-M)ₙ.

In accordance with the above-mentioned definition of HAS, the hydroxyalkyl starch derivative preferably comprises at least one structural unit according to the following formula (I)

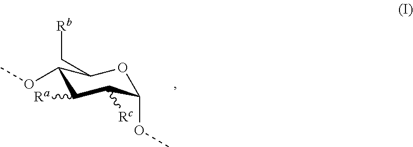

wherein at least one of R^{a}, R^{b} or R^{c} comprises the functional group Z¹ and wherein —R^{a}, —R^{b} and —R^{c} are, independently of each other, selected from the group consisting of —O-HAS″, —[O—(CR^{w}R^{x})—(CR^{y}R^{z})]ₓ—OH, —[O—(CR^{w}R^{x})—(CR^{y}R^{z})]_{y}—[F¹]ₚ-[L¹]_{q}—[F²]ᵣ-[L²]ᵥ-Z¹ wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, F¹ is a functional group, L¹ and L² are both linking moieties, F² is a functional group, and integers p, q, r and v are, independently of each other 0 or 1, with the proviso that in case p is 0, q is 0, and Z¹ is a functional group which is capable of being linked to a carbonyl group of a further compound, in particular to a carbonyl group comprised in the cytotoxic agent.

The term “capable of being linked to a carbonyl group” as used in the context of the present invention is denoted to mean a functional group which is reactive towards or may be reacted with a carbonyl group of a further compound, thereby forming a respective linkage with the carbonyl C atom of the further compound.

In particular, a hydroxyalkyl starch derivative which comprises at least one structural unit according to the following formula (I)

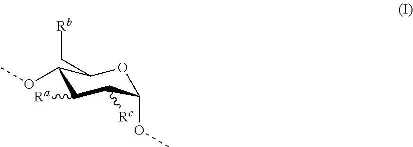

has preferably a structure according to the following formula (IV)

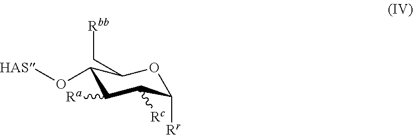

wherein R^{r} is —O-HAS″ or, in case the ring structure of formula (IV) shows the terminal saccharide unit of HAS, R^{r} is —OH, and wherein HAS″ is a remainder of the hydroxyalkyl starch derivative.

Analogously to the above-discussed definition of the term “HAS” in the context of the hydroxyalkyl starch as such, the term “remainder of the hydroxyalkyl starch derivative” is denoted to mean a linear or branched chain of the hydroxyalkyl starch derivative, being linked to the oxygen groups shown in formula (IV) or being comprised in the residues R^{a}, R^{b} or R^{c} of formula (I), wherein said linear or branched chains comprise at least one structural unit according to formula (I)

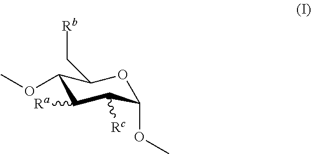

wherein at least one of R^{a}, R^{b} or R^{c} comprises the functional group Z¹ and/or one or more structural units of the formula (Ib)

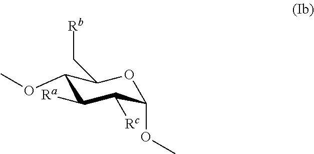

wherein R^{a}, R^{b} and R^{c} are, independently of each other, selected from the group consisting of —O-HAS″ and —[O—(CR^{w}R^{x})—(CR^{y}R^{z}]ₓ—OH, wherein R^{w}, R^{x}, R^{y}, R^{z} are as described above.

In case the hydroxyalkyl starch derivative has a linear starch backbone, none of R^{a}, R^{b} or R^{c} comprises a further group —O-HAS″. In case at least one of R^{a}, R^{b} or R^{c} is —O-HAS″, the hydroxyalkyl starch derivative comprises at least one branching point.

In particular, in case, the structural unit is the reducing sugar moiety of the hydroxyalkyl starch derivative, the terminal structural unit has a structure according to the following formula (Ia)

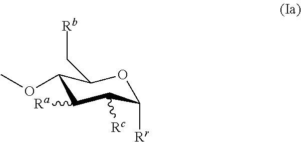

wherein R^{r} is preferably —OH. Besides, residue R^{r} may also comprise the functional group Z¹. In case, at least one of R^{a}, R^{b} or R^{c} of at least one structural unit according to the formula (I) is —[O—(CR^{w}R^{x})—(CR^{y}R^{z})]_{y}—[F¹]ₚ-[L¹]_{q}—[F²]ᵣ-[L²]ᵥ-Z¹, R^{r} of the corresponding reducing sugar moiety may have the structure: —[F¹]ₚ-[L¹]_{q}—[F²]ᵣ-[L²]ᵥ-Z¹.

In the above-mentioned formula (Ia), the bond “ [symbol] ” represents a bond with non-defined stereochemistry, i.e. this term represents a bond encompassing both possible stereochemistries. Preferably, the stereochemistry in most building blocks, preferably in all building blocks of the HAS derivative is defined according to the formulas (Ib) and (IVa)

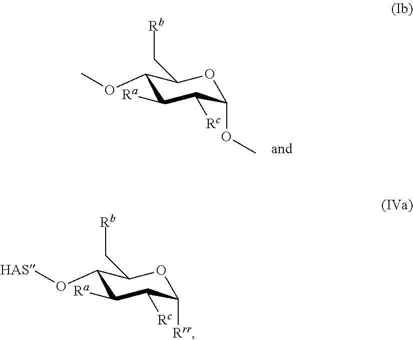

respectively.

According to a preferred embodiment of the present invention, the hydroxyalkyl starch (HAS) derivative is a hydroxyethyl starch (HES) derivative.

Therefore, the present invention also describes a hydroxyalkyl starch derivative as described above, and a method for preparing said hydroxyalkyl starch derivative, and a conjugate comprising said hydroxyalkyl starch derivative and a cytotoxic agent, and a conjugate obtained or obtainable by the above-mentioned method wherein the conjugate comprises said hydroxyalkyl starch derivative and a cytotoxic agent, wherein the hydroxyalkyl starch derivative is a hydroxyethyl starch derivative.

Accordingly, in case the hydroxyalkyl starch (HAS) is hydroxyethyl starch (HES), the HAS derivative preferably comprises at least one structural unit, preferably 3 to 200 structural units, preferably 3 to 100 structural units, according to the following formula (I)

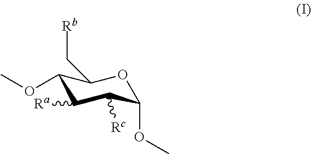

wherein R^{a}, R^{b} and R^{c} are independently of each other selected from the group consisting of —O-HAS″, —[O—CH₂—CH₂]ₛ—OH and —[O—CH₂—CH₂]ₜ—[F¹]ₚ-[L¹]_{q}—[F²]ᵣ-[L²]ᵥ-Z¹, wherein at least one R^{a}, R^{b} and R^{c} is —[O—CH₂—CH₂]ₜ—[F¹]ₚ-[L¹]_{q}—[F²]ᵣ-[L²]ᵥ-Z¹, wherein s in the range of from 0 to 4, wherein t is in the range of from 0 to 4.

### The Amount of Functional Groups Z¹ Present in the Hydroxyalkyl Starch Derivative

As regards the amount of functional groups Z¹ present in a given hydroxyalkyl starch derivative, preferably 0.3% to 4% of all residues R^{a}, R^{b} and R^{c} present in the hydroxyalkyl starch derivative contain the functional group Z¹.

More preferably, 0.4% to 3% of all residues R^{a}, R^{b} and R^{c} present in the hydroxyalkyl starch derivative have the structure —[O—(CR^{w}R^{x})—(CR^{y}R^{z})]_{y}—[F¹]ₚ-[L¹]_{q}—[F²]ᵣ-[L²]ᵥ-Z¹, preferably the structure —[O—CH₂—CH₂]ₜ—[F¹]ₚ-[L¹]_{q}—[F²]ᵣ-[L²]ᵥ-Z¹.

### The Term “Residue of the Hydroxyalkyl Starch Derivative”

The term “residue of the hydroxyalkyl starch derivative” (HAS′) refers to a hydroxyalkyl starch derivative being incorporated into a hydroxyalkyl starch conjugate. Within the meaning of the present invention the term “a conjugate comprising a hydroxyalkyl starch derivative” thus refers to a conjugate comprising a residue of a hydroxyalkyl starch derivative being incorporated into the conjugate and thus being linked to the residue of the cytotoxic agent M comprised in the conjugate, the conjugate having a structure according the following formula

HAS′(-M)ₙ.

Upon incorporation into the conjugate, the hydroxyalkyl starch derivative is coupled via at least one of its functional groups Z¹ to the cytotoxic agent, as described hereinabove and hereinunder, thereby forming a covalent linkage via a functional group X between the residue of the hydroxyalkyl starch derivative and the carbonyl carbon atom of M derived from the carbonyl group present in M.

In accordance with the above-mentioned definition of the hydroxyalkyl starch derivative, the residue of the hydroxyalkyl starch derivative preferably comprises at least one structural unit according to the following formula (I)

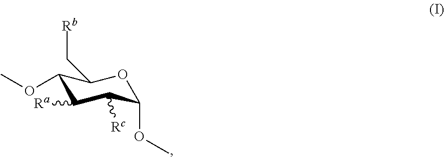

wherein —R^{a}, —R^{b} and —R^{c} are, independently of each other, selected from the group consisting of —O-HAS″, —[O—(CR^{w}R^{x})—(CR^{y}R^{z})]ₓ—OH and —[O—(CR^{w}R^{x})—(CR^{y}R^{z})]_{y}—[F¹]ₚ-[L¹]_{q}—[F²]ᵣ-[L²]ᵥ-X—, and wherein at least one of R^{a}, R^{b} or R^{c} comprises the functional group —[O—(CR^{w}R^{x})—(CR^{y}R^{z})]_{y}—[F¹]ₚ-[L¹]_{q}—[F²]ᵣ-[L²]ᵥ-X—, and wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, F¹ is a functional group, L¹ and L² are both linking moieties, F² is a functional group, and integers p, q, r and v are, independently of each other 0 or 1, with the proviso that in case p is 0, q is 0, and X is a functional group which is linked to the residue of the cytotoxic agent M, as described above.

Besides the at least one structural unit according to formula (I)

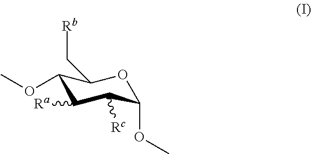

wherein at least one of R^{a}, R^{b} or R^{c} comprises the functional group —X—, preferably the structural unit —[O—(CR^{w}R^{x})—(CR^{y}R^{z})]_{y}—[F¹]ₚ-[L¹]_{q}—[F²]ᵣ-[L²]ᵥ-X—, the residue of the hydroxyalkyl starch preferably comprises one or more structural units of the formula (Ib)

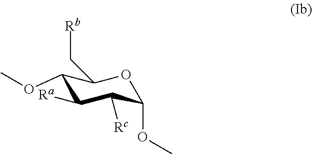

wherein R^{a}, R^{b} and R^{c} are, independently of each other, selected from the group consisting of —O-HAS″ and —[O—(CR^{w}R^{x})—(CR^{y}R^{z})]ₓ—OH.

As disclosed above, preferably 0.3% to 4% of all residues R^{a}, R^{b} and R^{c} present in the hydroxyalkyl starch derivative contain the functional group Z¹. Further, preferably all functional groups Z¹ being present in a given hydroxyalkyl starch derivative are coupled according to the coupling reaction of step (b) as defined hereinabove, thereby forming the covalent linkage via the functional group X to M. Consequently, preferably 0.3% to 4% of all residues R^{a}, R^{b} and R^{c} present in the residue of the hydroxyalkyl starch derivative being comprised in the conjugate of the invention contain the functional group X.

However, in case the hydroxyalkyl starch derivative comprises at least two functional groups Z¹, it may be possible that in step (b) not all of these functional groups Z¹ are coupled to the cytotoxic agent. Thus, embodiments are encompassed in which not all functional groups are coupled to the cytotoxic agent. The residue of the hydroxyalkyl starch derivative present in the conjugate of the invention may thus comprise at least one unreacted functional group Z¹. All conjugates mentioned hereinunder and above, may comprise such unreacted functional groups.

To avoid possible side effects due to the presence of such unreacted functional groups Z¹, the hydroxyalkyl starch conjugate may be further reacted with a suitable compound allowing for capping Z¹ with a reagent D*. However, preferably no such capping step is carried out.

As regards the amount of functional groups X being linked to M present in a given hydroxyalkyl starch conjugate, preferably at least 20%, more preferably at least 30%, more preferably at least 40%, most preferably at least 50%, of all functional groups X present in the conjugate of the invention are linked to M. Accordingly, preferably less than 80%, more preferably less than 70%, more preferably less than 60%, most preferably less than 50%, of all residues R^{a}, R^{b} and R^{c} present in a given hydroxyalkyl starch conjugate contain an unreacted group Z¹.

### Substitution Pattern: Molar Substitution (MS) and Degree of Substitution (DS)

HAS, in particular HES, is mainly characterized by the molecular weight distribution, the degree of substitution and the ratio of C₂:C₆ substitution. There are two possibilities of describing the substitution degree.

The degree of substitution (DS) of HAS is described relatively to the portion of substituted glucose monomers with respect to all glucose moieties.

The substitution pattern of HAS can also be described as the molar substitution (MS), wherein the number of hydroxyethyl groups per glucose moiety is counted.

In the context of the present invention, the substitution pattern of the hydroxyalkyl starch (HAS), preferably HES, is referred to as MS, as described above, wherein the number of hydroxyalkyl groups present per sugar moiety is counted (see also Sommermeyer et al., 1987, Krankenhauspharmazie, 8(8): 271-278, in particular page 273). The MS is determined by gaschromatography after total hydrolysis of the hydroxyalkyl starch molecule.

The MS values of the respective hydroxyalkyl starch, in particular hydroxyethyl starch starting materials, are given since it is assumed that the MS value is not affected during the derivatization procedures as well as during the coupling step of the present invention.

The MS value corresponds to the degradability of the hydroxyalkyl starch via alpha-amylase. The higher the MS value, the lower the degradability of the hydroxyalkyl starch. It was surprisingly found that the MS of the hydroxyalkyl starch derivative present in the conjugates according to the invention should preferably be in the range of from 0.6 to 1.5 to provide conjugates with advantageous properties. Without wanting to be bound to any theory, it is believed that a MS in the above mentioned range combined with the specific molecular weight range of the conjugates results in conjugates with an optimized enrichment of the cytotoxic agent in the tumor and/or residence time in the plasma allowing for a controlled release of the cytotoxic agent prior to the degradation of the polymer and the subsequent removal of polymer fragments through the kidney.

According to a preferred embodiment of the present invention, the molar substitution (MS) is in the range of from 0.70 to 1.45, more preferably in the range of 0.80 to 1.40, more preferably in the range of from 0.85 to 1.35, more preferably in the range of from 0.90 to 1.35, such as 0.90, 0.95, 1.0, 1.05, 1.1, 1.15, 1.2, 1.25, 1.3 or 1.35.

Thus, the present invention also relates to a method for preparing a conjugate comprising a hydroxyalkyl starch derivative and a cytotoxic agent, as described above, and a conjugate obtained or obtainable by said method, wherein the hydroxyalkyl starch derivative has a molar substitution MS in the range of from 0.70 to 1.45, more preferably of from 0.80 to 1.40, more preferably of from 0.85 to 1.35, more preferably of from 0.90 to 1.35. Likewise, the present invention also relates to a hydroxyalkyl starch (HAS) conjugate comprising a hydroxyalkyl starch derivative and a cytotoxic agent, as described above, wherein the hydroxyalkyl starch derivative has a molar substitution MS in the range of from 0.70 to 1.45, more preferably of from 0.80 to 1.40, more preferably of from 0.85 to 1.35, more preferably of from 0.90 to 1.35. Likewise, the present invention relates to a pharmaceutical composition comprising a hydroxyalkyl starch conjugate, as described above, or a hydroxyalkyl starch conjugate obtained or obtainable by the above described method, wherein the hydroxyalkyl starch derivative has a molar substitution MS in the range of from 0.70 to 1.45, more preferably of from 0.80 to 1.40, more preferably of from 0.85 to 1.35, more preferably of from 0.90 to 1.35.

As far as the ratio of C₂:C₆ substitution is concerned, i.e. the degree of substitution (DS) of HAS, said substitution is preferably in the range of from 2 to 20, more preferably in the range of from 2 to 15 and even more preferably in the range of from 3 to 12, with respect to the hydroxyalkyl groups.

### Mean Molecular Weight MW (or M_{w})

HAS and in particular HES compounds are present as polydisperse compositions, wherein each molecule differs from the other with respect to the polymerization degree, the number and pattern of branching sites, and the substitution pattern. HAS and in particular HES is therefore a mixture of compounds with different molecular weight. Consequently, a particular HAS and in particular a HES is determined by average molecular weight with the help of statistical means.

In this context the number average molecular weight is defined by equation 1:

$${\overline{M}}_{n} = \frac{{\sum }_{i}^{ } {n}_{i} \cdot {M}_{i}}{{\sum }_{i}^{ } {n}_{i}} \qquad ( 1 )$$

where nᵢ is the number of molecules of species i of molar mass Mᵢ. M̅ₙ indicates that the value is an average, but the line is normally omitted by convention.

M_{w} is the weight average molecular weight, defined by equation 2:

$${\overline{M}}_{w} = \frac{{\sum }_{i}^{ } {n}_{i} \cdot {M}_{i}^{2}}{{\sum }_{i}^{ } {n}_{i} {M}_{i}} \qquad ( 2 )$$

where nᵢ is the number of molecules of species i of molar mass Mᵢ and M̅_{w} indicates that the value is an average, but the line is normally omitted by convention.

Preferably, the hydroxyalkyl starch derivative, in particular the hydroxyethyl starch derivative comprised in the conjugate, as described above, has a mean molecular weight MW (weight mean) above the renal threshold.

The renal threshold is determined according to the method described by Waitzinger et al. (Clin. Drug Invest. 1998; 16: 151-160) and reviewed by Jungheinrich et al. (Clin. Pharmacokinet. 2006; 44(7): 681-699). Preferably, the renal threshold is denoted to mean a mean molecular weight MW above 40 kDa.

More preferably, the hydroxyalkyl starch derivative, in particular the hydroxyethyl starch derivative comprised in the conjugate, as described above, has a mean molecular weight MW above 45 kDa, more preferably above 50 kDa, more preferably above 60 kDa.

More preferably, the hydroxyalkyl starch derivative, in particular the hydroxyethyl starch derivative comprised in the conjugate, as described above, has a mean molecular weight MW above 60 kDa.

More preferably the hydroxyalkyl starch derivative, in particular the hydroxyethyl starch derivative, according to the invention, has a mean molecular weight MW (weight mean) in the range of from 80 to 1200 kDa, more preferably in the range of from 90 to 800 kDa.

The term “mean molecular weight” as used in the context of the present invention relates to the weight as determined according to MALLS (multiple angle laser light scattering) GPC method as described in example 1.9.

Therefore, the present invention also relates to a method as described above, for preparing a hydroxyalkyl starch derivative, as well as to a method for preparing a hydroxyalkyl starch conjugate, wherein the hydroxyalkyl starch derivative has a mean molecular weight MW above the renal threshold, preferably a MW greater than or equal to 60 kDa, more preferably in the range of from 80 to 1200 kDa, preferably in the range of from 90 to 800 kDa. Likewise, the present invention relates to a hydroxyalkyl starch conjugate, as described above, comprising a hydroxyalkyl starch derivative, as well as to a hydroxyalkyl starch conjugate obtained or obtainable by the above-mentioned method, wherein the hydroxyalkyl starch derivative has a mean molecular weight MW above the renal threshold, preferably a MW greater than or equal to 60 kDa, more preferably a mean molecular weight MW in the range of from 80 to 1200 kDa, more preferably in the range of from 90 to 800 kDa.

According to an especially preferred embodiment, the hydroxyalkyl starch derivative has a MS in the range of from 0.70 to 1.45 and a mean molecular weight MW in the range of from 80 to 1200 kDa, more preferably a molar substitution MS in the range of from 0.80 to 1.40 and a mean molecular weight MW in the range of from 90 to 800 kDa, more preferably a molar substitution in the range of from 0.85 to 1.35, more preferably a mean molecular weight MW in the range of from 90 to 800 kDa and a MS in the range of from 0.90 to 1.35.

As regards integer n, as described above and below, according to a preferred embodiment of the present invention, n is in the range of from 3 to 200, preferably of from 3 to 100.

### Drug Loading

The amount of M, present in the conjugates of the invention, can further be described by the drug loading (also: drug content). The “drug loading” as used in the context of the present invention is calculated as the mean molecular weight of the cytotoxic agent measured in mg drug, i.e. cytotoxic agent, per 1 g of the conjugate.

The drug loading is determined by measuring the absorbance of M (thus the cytotoxic agent bound to HAS) at a specific wavelength in a stock solution, and calculating the content using the following equation (Lambert Beer's law):

$${c}_{drug} [ μmol \text{/} {cm}^{3} ] = \frac{( A - {A}^{0} )}{ɛ * d}$$

where ε is the extinction coefficient of the cytotoxic agent at the specific wavelength, which is obtained from a calibration curve of the cytotoxic agent dissolved in the same solvent which is used as in the stock solution (given in cm²/μmol), at the specific wavelength, A is the absorption at this specific wavelength, measured in a UV-VIS spectrometer, A⁰ is the absorption of a blank sample and d the width of the cuvette (equals the slice of absorbing material in the path of the beam, usually 1 cm). The appropriate wavelength for the determination of drug loading is derived from a maximum in the UV-VIS-spectra, preferably at wavelengths above 230 nm.

With a known concentration of conjugate in the sample (c_{conjugate}) and the concentration of drug in the sample determined by Lambert Beer's law, the loading in micromol/g can be calculated according to the following equation:

$$Loading [ μmol \text{/} g ] = \frac{1000 * {c}_{drug} [ μmol \text{/} ml ]}{{c}_{conjugate} [ mg \text{/} ml ]}$$

The loading in mg/g may finally be determined taking into account the molecular weight of the cytotoxic agent as shown in the following equation:

Loading[mg/g]=Loading[μmol/g]*MW_{drug}[μg/μmol]1000

As regards the drug loading, according to a preferred embodiment of the present invention, the drug loading of the conjugates is preferably in the range of from 20 to 600 micromol/g, more preferably in the range of from 30 to 400 micromol/g, more preferably in the range of from 40 to 300 micromol/g, and most preferably in the range of from 55 to 240 micromol/g (-L-M).

### The Cytotoxic Agent

The term “cytotoxic agent” as used in the context of the present invention refers to natural or synthetic substances, which inhibit the cell growth or the cell division in vivo. The term is intended to include chemotherapeutic agents, antibiotics and toxins such as enzymatically active toxins of bacterial, fungal, plant or animal origin, or fragments thereof.

The term “residue of the cytotoxic agent M” as used in the context of the present invention refers to the cytotoxic agent being linked to the hydroxyalkyl starch derivative via a single or double bond, preferably a double bond, said group being derived from the reaction of X with the carbonyl group present in the cytotoxic agent.

Preferably, the term “cytotoxic agent” is a natural or synthetic substance which inhibits the cell growth or the cell division of a tumor in vivo. Most preferably, the cytotoxic agent is a chemotherapeutic agent. The therapeutic use of these preferred cytotoxic agents, most preferably of the chemotherapeutic agents, is based on the difference in the rate of cell division and cell growth of tumor cells compared to normal cells. Among others, tumor cells differ from normal cells in that tumor cells are no longer subject to physiological growth control and therefore have an increased rate of cell division. Since the toxic activity of cytotoxic agents is usually primarily directed against proliferating cells, such cytotoxic agents can be used for inhibiting a development or progression of a neoplasm in vivo, particularly a malignant (cancerous) lesion, such as a carcinoma, sarcoma, lymphoma, or leukemia. Inhibition of metastasis is frequently also a property of the cytotoxic agents encompassed by the present invention.

With respect to the chemistry used in the context of the present invention, any cytotoxic agent, preferably any chemotherapeutic agent, known to those skilled in the art can be incorporated into the conjugates according to the present invention provided that this cytotoxic agent, preferably the chemotherapeutic agent, comprises at least one carbonyl group.

Within the meaning of the present invention, the term “carbonyl group” is denoted to mean an aldehyde, keto or hemiacetal group.

Preferably the cytotoxic agent is an agent for the treatment of cancer.

Preferably the at least one carbonyl group containing cytotoxic agent is selected from the group consisting of tubulin interacting drugs, such as tubulin inhibitors or tubulin stabilizers (such as taxanes, members of the epothilone family (epothilone A-F, dehydelone, ixabepilone, sagopilone, KOS-862, BMS-310705) and taccalonolide, topoisomerase I inhibitors, topoisomerase II inhibitors, DNA intercalators (such as mitoxantrone), protein kinase inhibitors such as rapamycin and analogues (temsirolimus, everolimus), antimetabolites, mitotic inhibitors such as everolimus, DNA damaging agents, anthracyclines (such as doxorubicin, epirubicin, daunorubicin, idarubicin, valrubicin, esorubicin, caminomycin, 4-demethoxy-4′-O-methyl doxorubicin, 4′-O-tetrahydropyranyl-doxorubicin, 3′-deamino-3′-(3″-cyano-4″-morpholinyl)doxorubicin,aclacinomycin), hormone analogs such as prednisone and other compounds showing anti-cancer activities, e.g. didemnin B and aplidine.

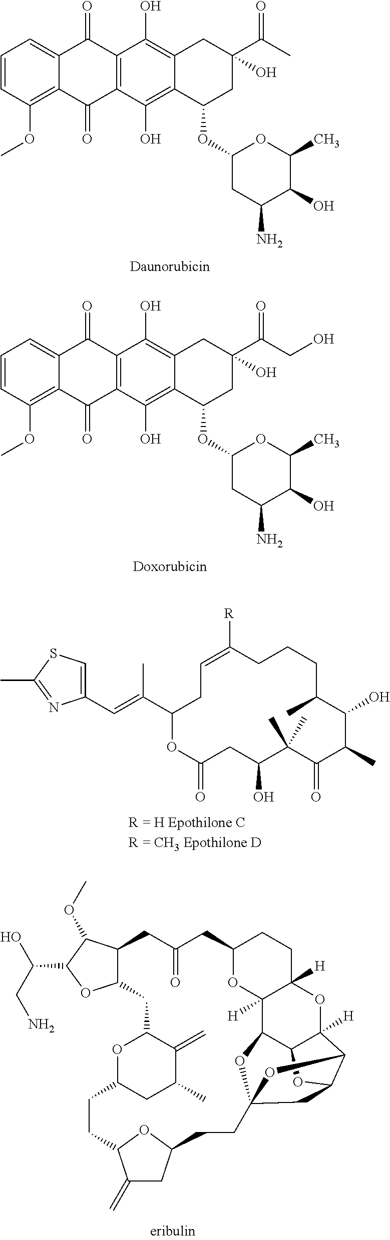

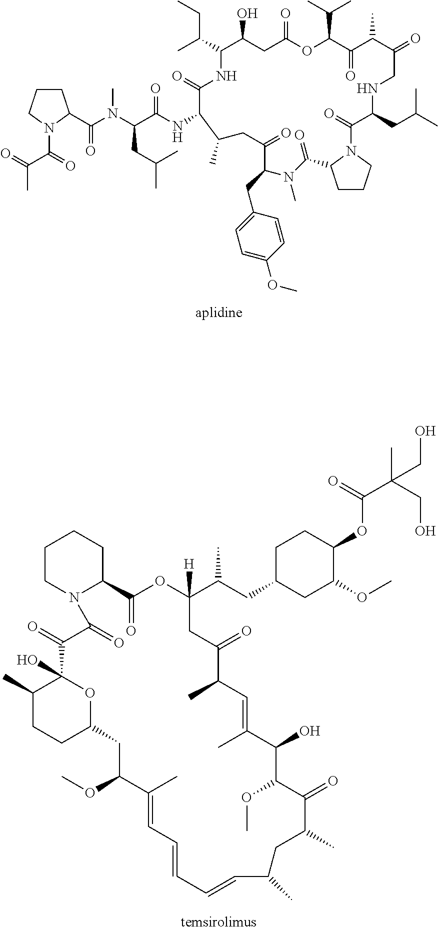

According to a preferred embodiment of the invention, the cytotoxic agent is an anthracycline.

Within the meaning of the present invention, the term “anthracycline” is denoted to encompass any cytotoxic agent comprising a tetracyclic quinoid ring structure:

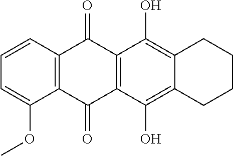 [SMILES: COc1cccc2c1C(=O)c1c(O)c3c(c(O)c1C2=O)CCCC3]

including but not limited to such agents in which the ring structure is coupled via a glycosidic linkage to a sugar moiety.

Thus, the term “anthracycline” encompasses, for example, agents such as daunorubicin, doxorubicin, epirubicin, idarubicin, valrubicin, esorubicin, caminomycin, 4-demethoxy-4′-O-methyl-doxorubicin, 4′-O-tetrahydropyranyl-doxorubicin, 3′-deamino-3′-(3″-cyano-4″-morpholinyl)doxorubicin, aclacinomycin and any cytotoxic analogs thereof.

Accordingly, the present invention preferably relates to a hydroxyalkyl starch conjugate as described above, as well as to a method for preparing a hydroxyalkyl starch conjugate and the respective conjugate obtained or obtainable by said method, the conjugate comprising a cytotoxic agent selected from the group consisting of daunorubicin, doxorubicin, epirubicin, idarubicin, valrubicin esorubicin, caminomycin, 4-demethoxy-4′-O-methyl doxorubicin, 4′-O-tetrahydropyranyl-doxorubicin, 3′-deamino-3′-(3″-cyano-4″-morpholinyl)doxorubicin, aclacinomycin and any cytotoxic analogs thereof, more preferably wherein the cytotoxic agent is selected from the group consisting of daunorubicin, doxorubicin, epirubicin, idarubicin, valrubicin, more preferably doxorubicin, epirubicin and idarubicin, most preferably doxorubicin and epirubicin.

More preferably, the cytotoxic agent is

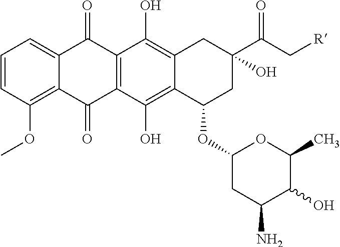

wherein R′ is preferably OH or CH₃.

Anthracyclines have been found to be effective anti-cancer agents. However, to date, their use is limited due to their low water solubility which requires acidic formulations, and/or their unspecific toxicity (especially cardiotoxicity) and/or their short residence time in the plasma. It is herein proposed that this drawback can be overcome by the conjugates according to the present invention, and thus, by linking the cytotoxic agents via a specific functional group to a hydroxyalkyl starch derivative, as described above.

Thus, preferably, the present invention also relates to a conjugate, as described above, as well as to a conjugate obtained or obtainable by a method, as described above, the conjugate having a structure according to the following formula

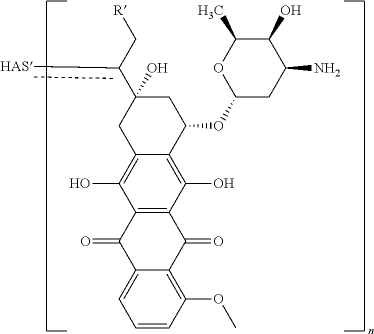

wherein R′ is —OH or —CH₃.

The following particularly preferred structure shall be mentioned:

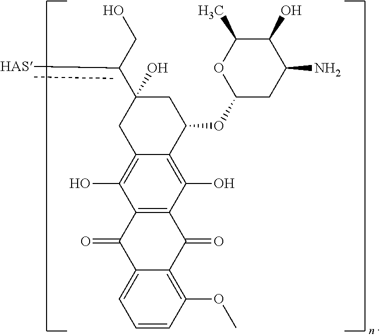

### Linking Group X

As described above, the residue of the hydroxyalkyl starch is linked via the functional group X to the carbon atom of at least one carbonyl group present in the cytotoxic agent, wherein the linkage is a cleavable linkage which is capable of being cleaved in vivo so as to release the cytotoxic agent.

The expression “cleavable linkage” refers to any linkage which can be cleaved physically or chemically and preferably releases the cytotoxic agent in unmodified form. Examples for physical cleavage may be cleavage by light, radioactive emission or heat, while examples for chemical cleavage include cleavage by redox reactions, hydrolysis, pH-dependent cleavage or cleavage by enzymes.

Especially the introduction of acid labile linkers is believed to be beneficial for drug targeting on the one hand since the pH of the extracellular regions of a tumor is generally lower than the physiological pH (L. E. Gerweck et al. Mol. Cancer. Ther. 2006; 5(5): 1275-9) and on the other hand since cellular uptake by endocytosis will release the drug in the acidic pH of the lysosomes.

According to a preferred embodiment of the present invention, the cleavable linker comprises one or more cleavable bonds, preferably pH dependent hydrolytically cleavable bonds, the cleavage, in particular the hydrolysis, of which releases the cytotoxic agent in vivo. Preferably, the bond between X and at least one carbon atom of M (which corresponds to the former carbonyl C atom of the respective cytotoxic agent) is cleaved in vivo.

Preferably the hydroxyalkyl starch derivative (HAS′) comprises at least one functional group X (also: the linking group X) being bound to the cytotoxic agent.

There are in principle no restrictions as to the nature of the functional group X provided that this group when linked with the carbonyl group of the cytotoxic agent forms a functional moiety capable of being cleaved in vivo, as described above.

Beside the functions which form functional groups comprising the structural unit —N═, this accounts, inter alia, also for groups which form together with the carbonyl group of M an acetal such as 1,2-diols or 1,3-diols. Such groups are also encompassed by the present invention.

Preferably, the at least one functional group X comprised in HAS has the structure -G′-NH—N═, and wherein G′ is selected from the group consisting of —Y^{G}—C(=G)-, —SO₂—, aryl, and heteroaryl, and wherein G is O or S, and wherein Y^{G} is —O—, —NH— or —NH—NH—.

Thus, in particular, the following structural units are described for —X—:

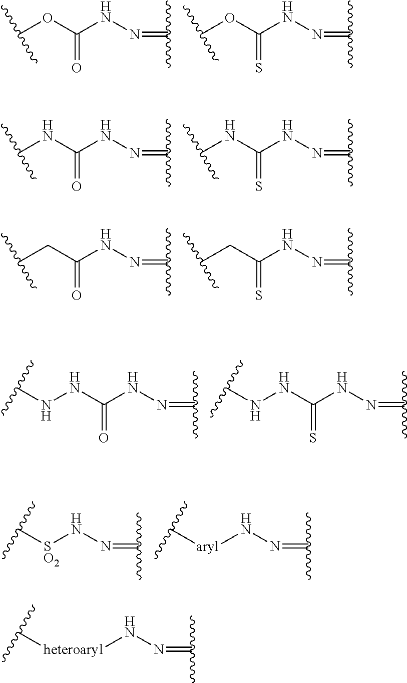

Thus, the present invention also relates to a conjugate as described above, as well as to a conjugate obtained or obtainable by the above described method, wherein the at least one functional group X comprised in HAS′ has the structure

-G′-NH—N═,

and wherein G′ is selected from the group consisting of —Y^{G}—C(=G)-, —SO₂—, aryl, and heteroaryl, wherein G is O or S, and wherein Y^{G} is —O—, —CH₂—, NH— or NH—NH—.

Within the meaning of the present invention a structure written in the following way -G′-NH—N═ may equally be written in the following way;

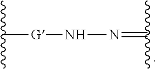

Within the meaning of the present invention, the term “aryl” refers to, but is not limited to, optionally suitably substituted 5- and 6-membered single-ring aromatic groups as well as optionally suitably substituted multicyclic groups, for example bicyclic or tricyclic aryl groups. The term “aryl” thus includes, for example, optionally suitably substituted phenyl groups or optionally suitably substituted naphthyl groups. Aryl groups can also be fused or bridged with alicyclic or heterocycloalkyl rings which are not aromatic so as to form a polycycle, e.g., benzodioxolyl or tetraline.

The term “heteroaryl” as used within the meaning of the present invention includes optionally suitably substituted 5- and 6-membered single-ring aromatic groups as well as substituted or unsubstituted multicyclic aryl groups, for example bicyclic or tricyclic aryl groups, comprising one or more, preferably from 1 to 4 such as 1, 2, 3 or 4, heteroatoms, wherein in case the aryl residue comprises more than 1 heteroatom, the heteroatoms may be the same or different. Such heteroaryl groups including from 1 to 4 heteroatoms are, for example, benzodioxolyl, pyrrolyl, furanyl, thiophenyl, thiazolyl, isothiazolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzimidazolyl, benzothiophenyl, methylenedioxyphenyl, napthyridinyl, quinolinyl, isoquinolinyl, indolyl, benzofuranyl, purinyl, deazapurinyl, or indolizinyl.

The term “substituted aryl” and the term “substituted heteroaryl” as used in the context of the present invention describes moieties having substituents replacing a hydrogen on one or more atoms, e.g. C or N, of an aryl or heteroaryl moiety. Again, there are in general no limitations as to the substituent. The substituents may be, for example, selected from the group consisting of alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, amino, acylamino, including alkylcarbonylamino, arylcarbonylamino, carbamoyl and ureido, amidino, nitro, imino, sulfhydryl, alkylthio, arylthio, thiocarboxylate, sulfate, alkylsulfinyl, sulfonate, sulfamoyl, sulfonamido, trifluoromethyl, cyano, azido, cycloalkyl such as e.g. cyclopentyl or cyclohexyl, heterocycloalkyl such as e.g. morpholino, piperazinyl or piperidinyl, alkylaryl, arylalkyl and heteroaryl. Preferred substituents of such organic residues are, for example, halogens, such as fluorine, chlorine, bromine or iodine, amino groups, hydroxyl groups, carbonyl groups, thiol groups and carboxyl groups.

In case X has the following structure

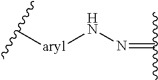

the aryl is preferably selected from the group consisting of phenyl, naphthyl or biphenyl. The respective residues may be further substituted as described above. Preferably the residues are unsubstituted.

In case X has the following structure

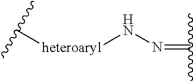

the heteroaryl is preferably selected from the group consisting of pyridyl, pyrimidyl and furanyl. The respective residues may be further substituted as described above. Preferably, the residues are unsubstituted.

According to a preferred embodiment, X is selected from the group consisting of

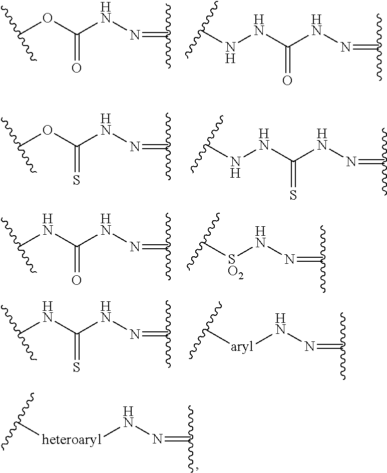

more preferably X is

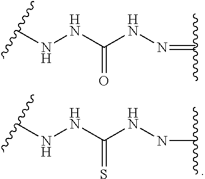 [SMILES: *NNC(=O)NN=* ; *NNC(=S)NN*]

Most preferably, the present invention relates to a conjugate as described above, as well as to a conjugate obtained or obtainable by the above described method, wherein the at least one functional group X comprised in HAS′ has the structure

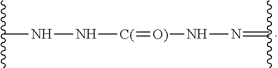

As described above, the hydroxyalkyl starch derivative comprising the functional group X preferably comprises at least one structural unit according to the following formula (I)

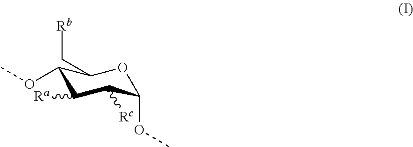

wherein at least one of R^{a}, R^{b} or R^{c} comprises the functional group X and wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}—[F¹]ₚ-[L¹]_{q}[F²]ᵣ-[L²]ᵥ-X—, and wherein at least one of R^{a}, R^{b} and R^{c} is —[O—(CR^{w}R^{x})—(CR^{y}R^{z})]_{y}—[F¹]ₚ-[L¹]_{q}—[F²]ᵣ-[L²]ᵥ-X—, wherein R^{w}, R^{x}, R^{y} and R^{z} are, independently of each other, selected from the group consisting of hydrogen and alkyl, y is an integer in the range of from 0 to 20, preferably in the range of from 0 to 4, x is an integer in the range of from 0 to 20, preferably in the range of from 0 to 4, wherein F¹ and F² are functional groups, L¹ is a linking moiety, L² is a linking moiety, and p, q, r and v are 0 or 1, with the proviso that in case p is 0, q is 0.

According to a preferred embodiment of the present invention, the hydroxyalkyl starch derivative is a hydroxyethyl starch derivative. Therefore, the present invention also describes a conjugate, comprising a residue of a hydroxyalkyl starch derivative, as described above, as well as a conjugate obtained or obtainable by the above-mentioned method, wherein the conjugate comprises a residue of a hydroxyethyl starch derivative and a cytotoxic agent, the residue of the HES derivative preferably comprises at least one structural unit according to the following formula (I)

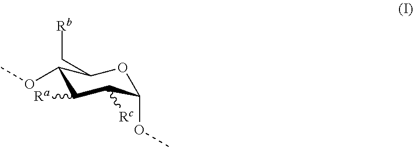

wherein R^{a}, R^{b} and R^{c} are independently of each other selected from the group consisting of —O-HAS″, —[O—CH₂—CH₂]ₛ—OH and —[O—CH₂—CH₂]ₜ—[F¹]ₚ-[L¹]_{q}—[F²]ᵣ-[L²]ᵥ-X—, and wherein s in the range of from 0 to 4, and wherein t is in the range of from 0 to 4, wherein at least one of R^{a}, R^{b} and R^{c} is —[O—CH₂—CH₂]ₜ—[F¹]ₚ-[L¹]_{q}—[F²]ᵣ-[L²]ᵥ-X—, and wherein HAS″ is a remainder of HAS (which means in this case the remainder of HES). According to a preferred embodiment of the present invention, this linkage X is obtained by coupling a hydroxyalkyl starch derivative being functionalized to comprise at least one functional group Z¹, as described above, to the cytotoxic agent, thereby forming the functional group X being linked to (the former carbonyl carbon atom of) M. Further preferred embodiments as to this method are described below.

As described above, according to a preferred embodiment of the present invention, at least one of R^{a}, R^{b} and R^{c} is —[F¹]ₚ-[L¹]_{q}—[F²]ᵣ-[L²]ᵥ-X—. Thus, the following structural units may be mentioned as preferred embodiments of the invention:

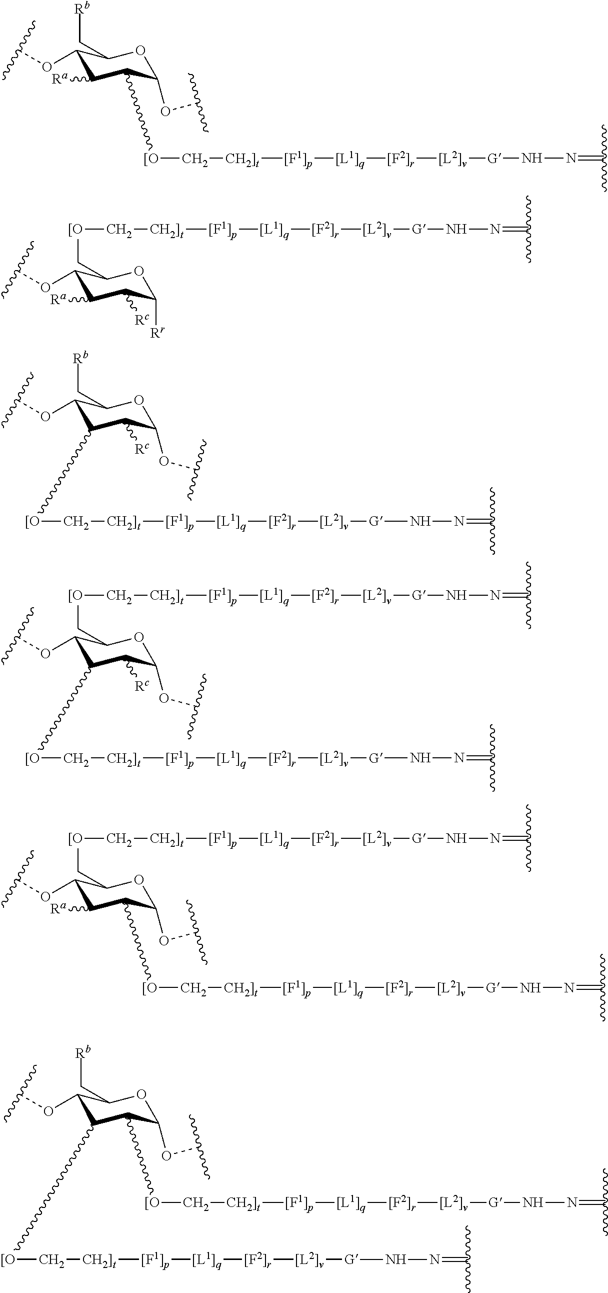

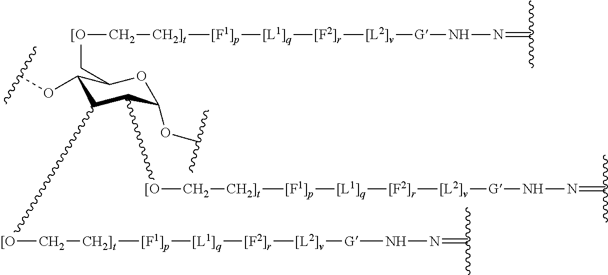

### The Functional Group F¹

F¹ is a functional group, which, if present, is selected from the group consisting of —O—, —S—, —NR^{Y7}— and —O—(C═Y⁶)—, wherein Y⁶ is selected from the group consisting of NR^{Y6}, O and S, more preferably Y⁶ is O, and wherein R^{Y6} is H or alkyl, preferably H, and wherein R^{Y7} is H or alkyl, preferably H, more preferably wherein F¹ is —O— or —O—(C═Y⁶)—, most preferably F¹, if present, is —O— or —O—C(═O)—.

Therefore, the present invention also describes a conjugate, comprising a hydroxyalkyl starch derivative, as described above, as well as a conjugate obtained or obtainable by the above-mentioned method, the hydroxyalkyl starch derivative preferably comprising at least one structural unit according to the following formula (I)

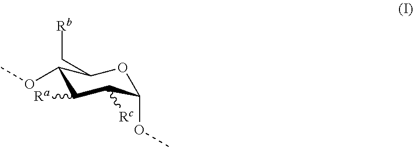

wherein at least one of R^{a}, R^{b} and R^{c} is —[O—(CR^{w}R^{x})—(CR^{y}R^{z})]_{y}—[O]ₚ-[L¹]_{q}—[F²]ᵣ-[L²]ᵥ-X— or —[O—(CR^{w}R^{x})—(CR^{y}R^{z})]_{y}—[O—C(═O)]ₚ-[L¹]_{q}—[F²]ᵣ-[L²]ᵥ-X—, preferably wherein at least one of R^{a}, R^{b} and R^{c} is —[O—CH₂—CH₂]ₜ—[O]ₚ-[L¹]_{q}—[F²]ᵣ-[L²]ᵥ-X— or —[O—CH₂—CH₂]ₜ-[—O—C(═O)]ₚ-[L¹]_{q}—[F²]ᵣ-[L²]ᵥ-X, more preferably wherein at least one of R^{a}, R^{b} and R^{c} is —[O—CH₂—CH₂]ₜ—[O]ₚ-[L¹]_{q}—[F²]ᵣ-[L²]ᵥ-G′-NH—N═ or —[O—CH₂—CH₂]ₜ—[—O—(C(═O)]ₚ-[L¹]_{q}[F²]ᵣ-[L²]ᵥ-G′-NH—N═.

### The Linking Moiety L¹

The term “linking moiety L¹” as used in this context of the present invention relates to any suitable chemical moiety bridging F¹ with the functional group F² or the linking moiety L² or the functional group X depending on whether r and/or v are 0 or 1.

In general, there are no particular restrictions as to the chemical nature of the spacer L¹ with the proviso that L¹ provides for a stable linkage between the functional group F¹ and the above mentioned building blocks. Preferably, L¹ is selected from the group consisting of alkyl, alkenyl, alkylaryl, arylalkyl, aryl, heteroaryl, alkylheteroaryl and heteroarylalkyl groups.

Within the meaning of the present invention, the term “alkyl” relates to non-branched alkyl residues, branched alkyl residues, cycloalkyl residues, as well as residues comprising one or more heteroatoms or functional groups, such as, by way of example, —O—, —S—, —NH—, —NH—C(═O)—, —C(═O)—NH—, and the like. The term also encompasses alkyl groups which are further substituted by one or more suitable substituent. The term “substituted alkyl” as used in this context of the present invention preferably refers to alkyl groups being substituted in any position by one or more substituents, preferably by 1, 2, 3, 4, 5 or 6 substituents, more preferably by 1, 2, or 3 substituents. If two or more substituents are present, each substituent may be the same or may be different from the at least one other substituent. There are in general no limitations as to the substituent. The substituents may be, for example, selected from the group consisting of aryl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, amino, acylamino, including alkylcarbonylamino, arylcarbonylamino, carbamoyl and ureido, amidino, nitro, imino, sulfhydryl, alkylthio, arylthio, thiocarboxylate, sulfate, alkylsulfinyl, sulfonate, sulfamoyl, sulfonamido, trifluoromethyl, cyano, azido, cycloalkyl, such as, e.g., cyclopentyl or cyclohexyl, heterocycloalkyl, such as, e.g., morpholino, piperazinyl or piperidinyl, alkylaryl, arylalkyl and heteroaryl. Preferred substituents of such organic residues are, for example, halogens, such as fluorine, chlorine, bromine or iodine, amino groups, hydroxyl groups, carbonyl groups, thiol groups and carboxyl groups.

The term “alkenyl” as used in the context of the present invention refers to unsaturated alkyl groups having at least one double bond. The term also encompasses alkenyl groups which are substituted by one or more suitable substituent.

The term “alkynyl” refers to unsaturated alkyl groups having at least one triple bond. The term also encompasses alkynyl groups which are substituted by one or more suitable substituent.

The term “alkylaryl” as used in the context of any linking moiety described in the present invention is denoted to mean a linking moiety having the structure alkyl-aryl-, thus being linked on one side via the alkyl group and on the other side via the aryl group, wherein this term is meant to also encompass linking moieties such as alkyl-aryl-alkyl- linking moieties. The term “alkylaryl group”, when used in the context of any substituent described hereinunder and above, is denoted to mean a residue being linked via the alkyl portion, said alkyl portion being further substituted with an aryl moiety.

The term “arylalkyl” as used in the context of any linking moiety described in the present invention is denoted to mean a linking moiety having the structure aryl-alkyl-, thus being linked on one side via the aryl group and on the other side via the alkyl group, wherein this term is meant to also encompass linking moieties such as aryl-alkyl-aryl- linking moieties. The term “arylalkyl group”, when used in the context of any substituent described hereinunder and above, is denoted to mean a residue being linked via the aryl portion, said aryl portion being further substituted with an alkyl moiety.

The term “alkylheteroaryl” as used in the context of any linking moiety described in the present invention is denoted to mean a linking moiety having the structure -alkyl-heteroaryl-, thus being linked on one side via the alkyl group and on the other side via the heteroaryl group, wherein this term is meant to also encompass linking moieties such as -alkyl-heteroaryl-alkyl- linking moieties. The term “alkylheteroaryl group”, when used in the context of any substituent described hereinunder and above, is denoted to mean a residue being linked via the alkyl portion, said alkyl portion being further substituted with a heteroaryl moiety.

The term “heteroarylalkyl” as used in the context of any linking moiety described in the present invention is denoted to mean a linking moiety having the structure -heteroaryl-alkyl-, thus being linked on one side via the heteroaryl group and on the other side via the alkyl group, wherein this term is meant to also encompass linking moieties such as -heteroaryl-alkyl-heteroaryl- linking moieties. The term “heteroarylalkyl group”, when used in the context of any substituent described hereinunder and above, is denoted to mean a residue being linked via the heteroaryl portion, said heteroaryl portion being further substituted with an alkyl moiety.

According to a preferred embodiment of the present invention, the linking moiety L¹ is a linking moiety comprising at least one structural unit according to the following formula —{[CR^{d}R^{f}]ₕ—[F⁴]ᵤ-[CR^{dd}R^{ff}]_{z}}ₐₗₚₕₐ-, wherein F⁴ is a functional group, preferably selected from the group consisting of —S—, —O— and —NH—, preferably wherein F⁴ is —O— or —S—, more preferably wherein F⁴ is —S—. The integer h is preferably in the range of from 1 to 20, more preferably of from 1 to 10, such as 1, 2, 3, 4, 5, 6, 7, 8, 9 or 10, more preferably of from 1 to 5, most preferably of from 1 to 3. Integer z is in the range of from 0 to 20, more preferably of from 0 to 10, such as 0, 1, 2, 3, 4, 5, 6, 7, 8, 9 or 10, more preferably of from 0 to 3, most preferably of from 0 to 2, such as 0, 1 or 2. Integer u is 0 or 1. Integer alpha is in the range of from 1 to 10, preferably of from 1 to 5, such as 1, 2, 3, 4, 5, more preferably 1 or 2. As regards residues R^{d}, R^{f}, R^{dd} and R^{ff}, these residues are, independently of each other, preferably selected from the group consisting of halogens, alkyl groups, H or hydroxyl groups. The repeating units of —[CR^{d}R^{f}]ₕ— may be the same or may be different. Likewise, the repeating units of —[CR^{dd}R^{ff}]_{z}— may be the same or may be different. Likewise in case integer alpha is greater than 1, the groups F⁴ in each repeating unit may be the same or may be different. Further, in case alpha is greater than 1, integer h in each repeating may be the same or may be different, integer z in each repeating may be the same or may be different and integer u in each repeating may be the same or may be different. Most preferably, R^{d}, R^{f}, R^{dd} and R^{ff} are, independently of each other, H, alkyl or hydroxyl.

According to one embodiment of the present invention, u and z are 0, the linking moiety L¹, thus, corresponds to the structural unit —[CR^{d}R^{f}]ₕ—.

According to an alternative embodiment of the present invention u is 1. According to this embodiment z is preferably greater than 0, preferably 1 or 2.

Thus, the following preferred structures for the linking moiety L¹ are mentioned by way of example: —{[CR^{d}R^{f}]ₕ—F⁴—[CR^{dd}R^{ff}]_{z}}ₐₗₚₕₐ- and —[CR^{d}R^{f}]ₕ—.

Thus, by way of the example, the following especially preferred linking moieties L¹ are mentioned:
—CH₂—,
—CH₂—CH₂—,
—CH₂—CH₂—CH₂—,
—CH₂—CH₂—CH₂—CH₂—,
—CH₂—CH₂—CH₂—CH₂—CH₂—,
—CH₂—CH₂—O—CH₂—CH₂—,
—CH₂—CH₂—O—CH₂—CH₂—O—CH₂—CH₂—,
—CH₂—CHOH—CH₂—,
—CH₂—CHOH—CH₂—O—CH₂—CHOH—CH₂—,
—CH₂—CH(CH₂OH)—
—CH₂—CH₂—CHOH—CH₂—,
—CH₂—CH₂—CH₂—CHOH—CH₂—.

According to one preferred embodiment, R^{d}, R^{f} and, if present, R^{dd} and R^{ff} are preferably H or hydroxyl, more preferably at least one of R^{d} and R^{f} of at least one repeating unit of —[CR^{d}R^{f}]ₕ— is —OH, wherein even more preferably, in this case, both R^{dd} and R^{ff} are H, if present. In particular, L¹ is selected from the group consisting of —CH₂—CHOH—CH₂—, —CH₂—CH(CH₂OH)—, —CH₂—CHOH—CH₂—O—CH₂—CHOH—CH₂—, —CH₂—CH₂—CHOH—CH₂—, —CH₂—CH₂—CH₂—CHOH—CH₂—, more preferably from the group consisting of —CH₂—CHOH—CH₂—, —CH₂—CH(CH₂OH)—, —CH₂—CHOH—CH₂—O—CH₂—CHOH—CH₂—, most preferably from the group consisting of —CH₂—CHOH—CH₂— and —CH₂—CH(CH₂OH)—.

Therefore, the present invention also describes a hydroxyalkyl starch derivative, comprised in a conjugate, as described above, as well as in a conjugate obtained or obtainable by the above-mentioned method, the hydroxyalkyl starch derivative comprising at least one structural unit according to the following formula (I)

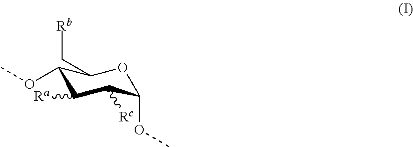

wherein at least one of R^{a}, R^{b} and R^{c} has a structure according to the following formula —[O—CH₂—CH₂]ₜ—[F¹]ₚ-[L¹]_{q}—[F²]ᵣ-[L²]ᵥ-X—, wherein L¹, if present, is selected from the group consisting of —CH₂—CHOH—CH₂—, —CH₂—CH(CH₂OH)—, —CH₂—CHOH—CH₂—O—CH₂—CHOH—CH₂—, —CH₂—CH₂—CHOH—CH₂—, —CH₂—CH₂—CH₂—CHOH—CH₂—, more preferably from the group consisting of —CH₂—CHOH—CH₂—, —CH₂—CH(CH₂OH)—, —CH₂—CHOH—CH₂—O—CH₂—CHOH—CH₂—, most preferably L¹ is —CH₂—CHOH—CH₂— or —CH₂—CH(CH₂OH)—.

### The Linking Moiety L²

In general, there are no particular restrictions as to the chemical nature of the linking moiety L². The term “linking moiety L²” as used in the context of the present application, relates to any suitable chemical moiety, if present, bridging F² and X, in case r is 1. Preferably, in case r is 0, L¹ is 0 as well.

L² is preferably a group selected from the group consisting of alkyl, alkenyl, alkylaryl, arylalkyl, aryl, heteroaryl, alkylheteroaryl and heteroarylalkyl. The respective residues may comprise one or more substituents as described above.

In particular, L² comprises at least one structural unit according to the following formula

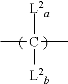

wherein L²ₐ and L²_{b} are independently of each other, H or an organic residue selected from the group consisting of alkyl, alkenyl, alkylaryl, arylalkyl, aryl, heteroaryl, alkylheteroaryl, heteroarylalkyl, hydroxyl and halogen, such as fluorine, chlorine, bromine, or iodine.

More preferably, L² has a structure according to the following formula

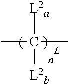

with L²ₐ and L²_{b} being selected from the group consisting of H, methyl or hydroxyl, with n^{L} being preferably in the range of from 1 to 8, more preferably of from 1 to 6, more preferably of from 1 to 4, more preferably of from 1 to 3, and most preferably of from 2 to 3. According to an even more preferred embodiment, the spacer L² consists of the structural unit according to the following formula

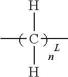

wherein integer n^{L} is in the range of from 1 to 8, more preferably of from 1 to 6, more preferably of from 1 to 4, more preferably of from 1 to 3, and most preferably of from 2 or 3. Therefore, according to a preferred embodiment of the present invention, L² has a structure selected from the group consisting of —CH₂—CH₂—CH₂—CH₂—CH₂—CH₂—, —CH₂—CH₂—CH₂—CH₂—CH₂—, —CH₂—CH₂—CH₂—CH₂—, —CH₂—CH₂—CH₂—, —CH₂—CH₂—, —CH₂—, —CH₂—(C₆H₄)—CH₂—, more preferably L² is selected from the group consisting of —CH₂—CH₂— and —CH₂—CH₂—CH₂—.

### The Functional Group F²

There are, in general, no particular restrictions as regards the chemical nature of the functional group F², provided that F² is capable of linking L² with L¹ or with F¹ or with the structural unit —[O—(CR^{w}R^{x})—(CR^{y}R^{z})]ₓ-, respectively.

Preferably, F² is a functional group selected from the group consisting of S—, —NH— and —NH—NH-T′-, wherein T′ is selected from the group consisting of —C(=T)-Y^{T}—, —SO₂—, aryl, and heteroaryl, and wherein T is O or S, and wherein Y^{T} is CH₂—, —O—, —NH— or —NH—NH—, preferably —O—, NH— or NH—NH—.

Thus, in particular, the following structural units are described for F²:

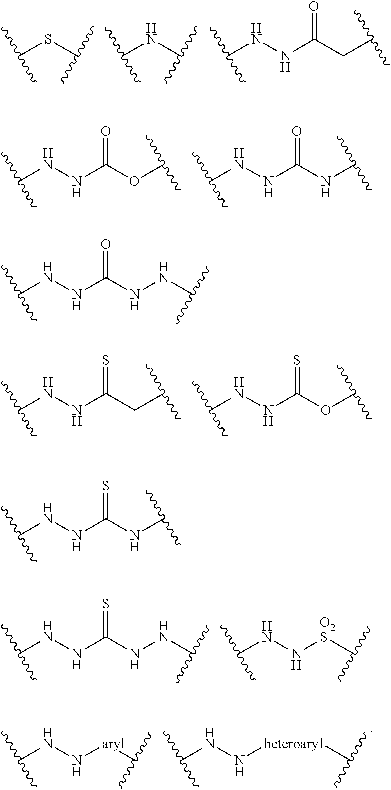

Thus, the present invention also describes a hydroxyalkyl starch derivative, comprised in a conjugate, as described above, as well as in a conjugate obtained or obtainable by the above mentioned method, the hydroxyalkyl starch derivative comprising at least one structural unit according to the following formula (I)

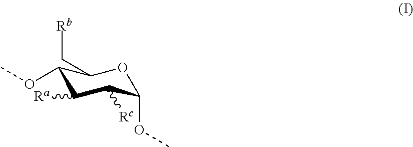

wherein at least one of R^{a}, R^{b} and R^{c} has a structure according to the following formula —[O—CH₂—CH₂]ₜ—[F¹]ₚ-[L¹]_{q}—[F²]ᵣ-[L²]ᵥ-X—, and wherein F² is a functional group selected from the group consisting of S—, —NH— and —NH—NH-T′-, wherein T′ is selected from the group consisting of —C(=T)-Y^{T}—, —SO₂—, aryl, and heteroaryl, and wherein T is O or S, and wherein Y^{T} is CH₂—, —O—, NH— or NH—NH—, preferably —O—, NH— or —NH—NH—.

In case F² has the following structure

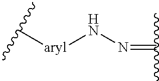

the aryl is preferably selected from the group consisting of phenyl, naphthyl or biphenyl. The respective residues may be further substituted as described above. Preferably, the residues are unsubstituted.

In case F² has the following structure

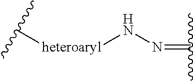

the heteroaryl is preferably selected from the group consisting of pyridyl, pyrimidyl and furanyl. The respective residues may be further substituted as described above. Preferably the residues are unsubstituted.

The functional group F² is suitably chosen, in particular depending on the functional group X being present in the hydroxyalkyl starch derivative.

Preferably, the functional group F² is selected from the group consisting of

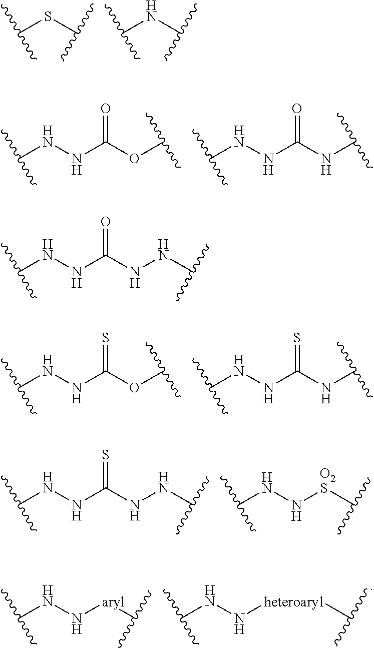

According to one preferred embodiment of the invention, F² is selected from the group consisting of

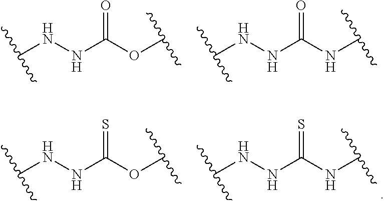 [SMILES: *NNC(=O)O* ; *NNC(=O)N* ; *NNC(=S)O* ; *NNC(=S)N*]

According to an alternative preferred embodiment, F² is —S—.

### Preferred Conjugates According to the Invention

According to a first embodiment, F¹ is —O—C(═O)—, thus, the hydroxyalkyl starch derivative comprised in the conjugate of the invention, comprises at least one structural unit according to formula (Ib),

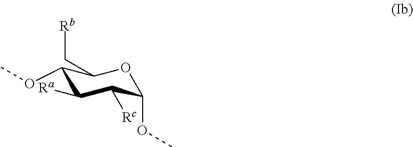

wherein R^{a}, R^{b} and R^{c} are independently of each other selected from the group consisting of —O—HAS″, —[O—CH₂—CH₂]ₛ—OH and —[O—CH₂—CH₂]ₜ—O—C(═O)-[L¹]_{q}—[F²]ᵣ-[L²]ᵥ-X—, and wherein s in the range of from 0 to 4, and wherein t is in the range of from 0 to 4, wherein at least one of R^{a}, R^{b} and R^{c} is —[O—CH₂—CH₂]ₜ—O—C(═O)-[L¹]_{q}—[F²]ᵣ-[L²]ᵥ-X—, and wherein HAS″ is a remainder of HAS′.

In this case, L¹ is preferably absent, i.e. q is 0. Thus, preferably at least one of R^{a}, R^{b} and R^{c} is —[O—CH₂—CH₂]ₜ—O—C(═O)—[F²]ᵣ-[L²]ᵥ-X—.

More preferably, in this case
(i) r and v are 0, and X has the structure

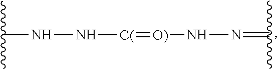

or
(ii) r and v are both 1, and X has the structure -G′—NH—N═, wherein G′ is selected from the group consisting of —Y^{G}—C(=G)-, —SO₂—, aryl, and heteroaryl, wherein G is O or S, and wherein Y^{G} is —O— or NH—,
   and F² has the structure —NH—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, NH—.

The hydroxyalkyl starch derivative being comprised in the above mentioned conjugate may comprise, in addition, the functional moiety —[F²]ᵣ-[L²]ᵥ-X— attached to the reducing end. In this case, n in the formula HAS′(-M)ₙ is at least 2 and the above mentioned terminal sugar has a structure according to the following formula (Ia)

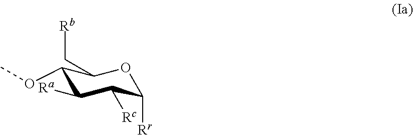

wherein R^{r} is —[F²]ᵣ-[L²]ᵥ-X—. This closed form, may be depending on e.g. the solvent, in equilibrium with the opened form as shown in the scheme below:

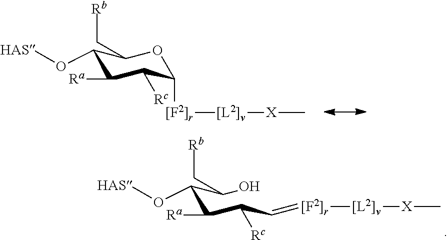

According to a second preferred embodiment, F¹ is —O—, i.e. the hydroxyalkyl starch derivative comprised in the conjugate of the invention, comprises at least one structural unit according to formula (Ib), wherein R^{a}, R^{b} and R^{c} are independently of each other selected from the group consisting of —O-HAS″, —[O—CH₂—CH₂]ₛ—OH and —[O—CH₂—CH₂]ₜ—O-[L¹]_{q}—[F²]ᵣ-[L²]ᵥ-X—, and wherein s in the range of from 0 to 4, and wherein t is in the range of from 0 to 4, wherein at least one of R^{a}, R^{b} and R^{c} is —[O—CH₂—CH₂]ₜ—O—[L¹]_{q}—[F²]ᵣ-[L²]ᵥ-X—, and wherein HAS″ is a remainder of HAS′. In this case, q is 1 and L¹ has a structure selected from the group consisting of —CH₂—CHOH—CH₂—, —CH₂—CH(CH₂OH)—, —CH₂—CHOH—CH₂—O—CH₂—CHOH—CH₂—, —CH₂—CH₂—CHOH—CH₂—, —CH₂—CH₂—CH₂—CHOH—CH₂—, more preferably from the group consisting of —CH₂—CHOH—CH₂—, —CH₂—CH(CH₂OH)—, —CH₂—CHOH—CH₂—O—CH₂—CHOH—CH₂—, more preferably from the group consisting of —CH₂—CHOH—CH₂— and —CH₂—CH(CH₂OH)—.

More preferably, in this case
(i) r and v are 0, and X has the structure

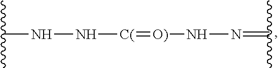

or
(ii) r and v are both 1, and X has the structure -G′-NH—N═, wherein G′ is selected from the group consisting of —Y^{G}—C(=G)-, —SO₂—, aryl, and heteroaryl, wherein G is O or S, and wherein Y^{G} is —O— or NH—,
   and F² has the structure —NH—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, or —NH—.

Again, also this hydroxyalkyl starch derivative being comprised in the above mentioned conjugate may comprise, in addition, the functional moiety —[F²]ᵣ-[L²]ᵥ-X— attached to the reducing end. In this case, n in the formula HAS′(-M)ₙ is at least 2 and the above mentioned terminal sugar has a structure according to the following formula (a)

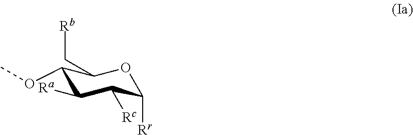

wherein R^{r} is —[F²]ᵣ-[L²]ᵥ-X—. This closed from, may be depending on e.g. the solvent, in equilibrium with the opened form as shown in the scheme below:

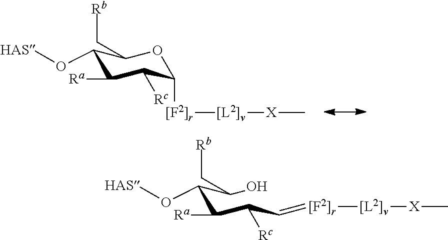

Moreover, the following preferred embodiment is described, wherein p and q are 0, and wherein the hydroxyalkyl starch derivative comprised in the conjugate of the invention, comprises at least one structural unit according to formula (I)

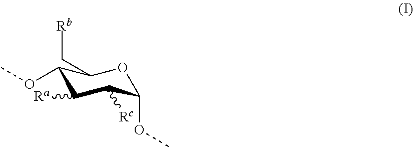

wherein R^{a}, R^{b} and R^{c} are independently of each other selected from the group consisting of —O—HAS″, —[O—CH₂—CH₂]ₛ—OH and —[O—CH₂—CH₂]ₜ—[F²]ᵣ-[L²]ᵥ-X—, and wherein s in the range of from 0 to 4, and wherein t is in the range of from 0 to 4, wherein at least one of R^{a}, R^{b} and R^{c} is —[O—CH₂—CH₂]ₜ—[F²]ᵣ-[L²]ᵥ-X—.

More preferably, in this case
(i) r and v are 0, and X has the structure

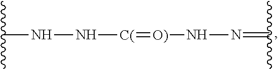

or
(ii) r and v are both 1, and X has the structure -G′—NH—N═, wherein G′ is selected from the group consisting of —Y^{G}—C(=G)-, —SO₂—, aryl, and heteroaryl, wherein G is O or S, and wherein Y^{G} is —O— or —NH—,
   and wherein F² has the structure —NH—NH-T′-, wherein T′ is selected from the group consisting of —C(=T)-Y^{T}—, —SO₂—, aryl, and heteroaryl, and wherein T is O or S, and wherein Y^{T} is —CH₂—, —O— or —NH—.

This especially preferred embodiment, may also comprise, in addition, the functional moiety —[F²]ᵣ-[L²]ᵥ-X— attached to the reducing end. In this case, n in the formula HAS′(-M)ₙ is at least 2 and the above mentioned terminal sugar has a structure according to the following formula (Ia):

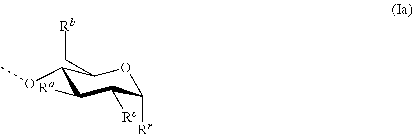

wherein R^{r} is —[F²]ᵣ-[L²]ᵥ-X—. This closed form, may be depending on, e.g., the solvent, in equilibrium with the opened form as shown in the scheme below:

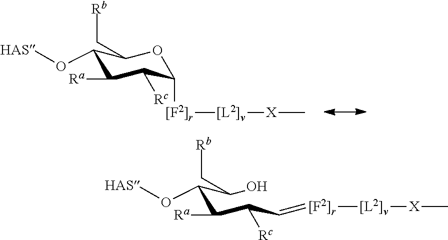

By way of example, without intention to limit the scope of the invention, in the following table, preferred conjugates of the invention are mentioned.

TABLE 1
Preferred conjugates according the invention
Structure: HAS′(—M)ₙ
wherein HAS′ comprises at least one structural unit according to formulas (I) or (Ib)
(I)

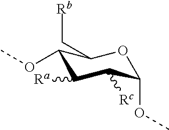

or
(Ib)

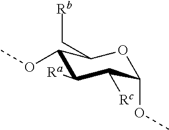

wherein at least one of R^{a}, R^{b} and R^{c} is —[O—CH₂—CH₂]ₜ—[F¹]ₚ—[L¹]_{q}—[F²]ᵣ—[L²]ᵥ—X—

| [F¹]ₚ | —[L¹]_{q} | —[F²]ᵣ | —[L²]ᵥ |
|---|---|---|---|
| —O— | —CH₂—CHOH—CH₂— | r is 0 | v is 0 |
| p is 1 | q is 1 | | |
| —O— | —CH₂—CH(CH₂OH)— | r is 0 | v is 0 |
| p is 1 | q is 1 | | |
| —O— | —CH₂—CHOH—CH₂— | —NH—NH—C(═O)— | —CH₂—CH₂—CH₂—CH₂—CH₂—CH₂—, |
| p is 1 | q is 1 | r is 1 | —CH₂—CH₂—CH₂—CH₂—CH₂—, |
| | | | —CH₂—CH₂—CH₂—CH₂—, |
| | | | —CH₂—CH₂—CH₂—, |
| | | | —CH₂—CH₂—, |
| | | | —CH₂— |
| | | | v is 1 |
| —O— | —CH₂—CH(CH₂OH)— | —NH—NH—C(═O)— | —CH₂—CH₂—CH₂—CH₂—CH₂—CH₂—, |
| p is 1 | q is 1 | r is 1 | —CH₂—CH₂—CH₂—CH₂—CH₂—, |
| | | | —CH₂—CH₂—CH₂—CH₂—, |
| | | | —CH₂—CH₂—CH₂—, |
| | | | —CH₂—CH₂—, |
| | | | —CH₂— |
| | | | v is 1 |
| p is 0 | q is 0 | r is 0 | v is 0 |
| p is 0 | q is 0 | —NH—NH—C(═O)— | —CH₂—CH₂—CH₂—CH₂—CH₂—CH₂—, |
| | | r is 1 | —CH₂—CH₂—CH₂—CH₂—CH₂—, |
| | | | —CH₂—CH₂—CH₂—CH₂—, |
| | | | —CH₂—CH₂—CH₂—, |
| | | | —CH₂—CH₂—, |
| | | | —CH₂— |
| | | | v is 1 |
| p is 0 | q is 0 | —S— | —CH₂—CH₂—CH₂—CH₂—CH₂—CH₂—, |
| | | r is 1 | —CH₂—CH₂—CH₂—CH₂—CH₂—, |
| | | | —CH₂—CH₂—CH₂—CH₂—, |
| | | | —CH₂—CH₂—CH₂—, |
| | | | —CH₂—CH₂—, |
| | | | —CH₂— |
| | | | v is 1 |
| —O—C(═O) | q is 0 | r is 0 | v is 0 |
| p is 1 | | | |

| | | Structural | |
|---|---|---|---|
| [F¹] ₚ | —X— | unit | M |
| —O— | —NH—NH—C(═O)—NH—N═ | Ib | daunorubicin, |
| p is 1 | | | doxorubicin, |
| | | | epirubicin, |
| | | | idarubicin, |
| | | | valrubicin |
| —O— | —NH—NH—C(═O)—NH—N═ | Ib | daunorubicin, |
| p is 1 | | | doxorubicin, |
| | | | epirubicin, |
| | | | idarubicin, |
| | | | valrubicin |
| —O— | —C(═O)—NH—N═ | Ib | doxorubicin |
| p is 1 | | | |
| —O— | —C(═O)—NH—N═ | Ib | doxorubicin |
| p is 1 | | | |
| p is 0 | —NH—NH—C(═O)—NH—N═ | I | doxorubicin |
| p is 0 | —C(═O)—NH—N═ | I | daunorubicin, |
| | | | doxorubicin, |
| | | | epirubicin, |
| | | | idarubicin, |
| | | | valrubicin |
| p is 0 | —C(═O)—NH—N═ | I | doxorubicin |
| —O—C(═O) | —NH—NH—C(═O)—NH—N═ | Ib | doxorubicin |
| p is 1 | | | |

### Synthesis of HAS Conjugates

As described above, the present invention also relates to a method for preparing a hydroxyalkyl starch (HAS) conjugate comprising a hydroxyalkyl starch derivative and a cytotoxic agent, said conjugate having a structure according to the following formula

HAS′(-M)ₙ

wherein M is a residue of a cytotoxic agent, wherein the cytotoxic agent comprises a carbonyl group, HAS′ is a residue of the hydroxyalkyl starch derivative comprising at least one functional group X, n is greater than or equal to 1, preferably in the range of from 3 to 200, preferably of from 3 to 100,
and wherein the cytotoxic agent is linked via the carbonyl function present in the cytotoxic agent to a functional group X comprised in the hydroxyalkyl starch derivative, wherein the linkage via the carbonyl function is a cleavable linkage, which is capable of being cleaved in vivo so as to release the cytotoxic agent,
said method comprising
(a) providing a hydroxyalkyl starch (HAS) derivative, said HAS derivative comprising a functional group Z¹; and providing a cytotoxic agent comprising a carbonyl group;
(b) coupling the HAS derivative to the cytotoxic agent, wherein the functional group Z¹ comprised in the hydroxyalkyl starch derivative is coupled directly to the carbonyl group of the cytotoxic agent thereby forming the functional group X (wherein this group is linked to the carbon atom of the former carbonyl group).

Hydroxyalkyl starches having the desired properties are preferably produced from waxy maize starch or potato starch by acidic hydrolysis and reaction with ethylene oxide and purification by ultrafiltration.

### The Functional Group Z¹

In the context of the present invention, Z¹ is the functional group of HAS′ capable of being reacted with the at least one carbonyl group present in the cytotoxic agent, wherein upon reaction of Z¹ with the carbonyl group, the functional group —X— is formed, with X being linked, preferably via a double bond, to the carbon atom of the at least one former carbonyl C atom present in M.

According to a preferred embodiment, Z¹ has the structure -G′-NH—NH₂, wherein G′ is selected from the group consisting of —Y^{G}—C(=G)-, —SO₂—, aryl, and heteroaryl, wherein G is O or S, and wherein Y^{G} is —CH₂—, —O—, —NH— or —NH—NH—. Thus, in particular, the following structural units are mentioned:

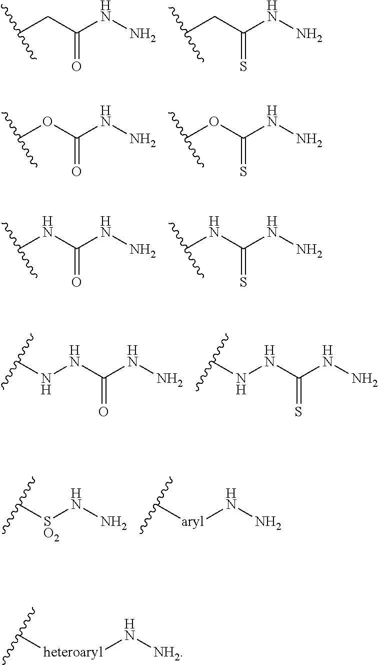

In case Z¹ has the following structure

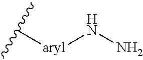

the aryl is preferably selected from the group consisting of phenyl, naphthyl or biphenyl. The respective residues may be further substituted as described above. Preferably, the residues are unsubstituted.

In case Z¹ has the following structure

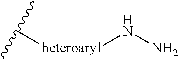

the heteroaryl is preferably selected from the group consisting of pyridyl, pyrimidyl and furanyl. The respective residues may be further substituted as described above. Preferably, the residues are unsubstituted.

According to a preferred embodiment, Z¹ is selected from the group consisting of

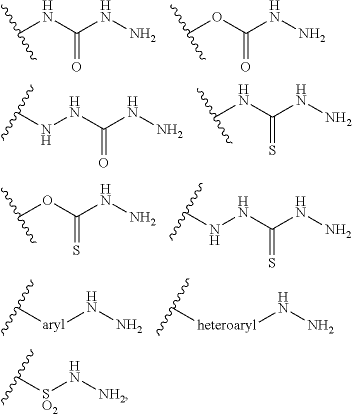

more preferably Z¹ is

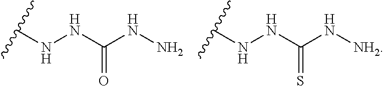 [SMILES: *NNC(=O)NN ; *NNC(=S)NN]

Most preferably, Z¹ comprised in HAS′ has the structure

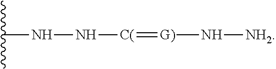

### Step (a)

As regards the provision of the hydroxyalkyl starch derivative according to step (a), as described above, step (a) preferably comprises the introduction of at least one functional group Z¹ into the hydroxyalkyl starch by
(i) coupling the hydroxyalkyl starch via at least one hydroxyl group comprised in HAS to at least one suitable linker comprising the functional group Z¹ or a precursor of the functional group Z¹, or
(ii) displacing at least one hydroxyl group comprised in HAS in a substitution reaction with a suitable linker comprising the functional group Z¹ or a precursor thereof.

The term “coupling via at least one hydroxyl group” as used hereinunder and above is denoted to mean a coupling, wherein the oxygen atom of the (former) hydroxyl group is coupled to a respective group of the at least one suitable linker.

The term “a precursor of the functional group Z¹” as used in the context of the present invention is denoted to mean a functional group which is capable of being transformed in at least one further step to give the functional group Z¹.

According to a preferred embodiment of the present invention, the present invention relates to a method for preparing a hydroxyalkyl starch conjugate, as described above, wherein the hydroxyalkyl starch derivative provided in step (a) comprises at least one structural unit, preferably 3 to 200 structural units, more preferably 3 to 100 structural units, according to the following formula (I)

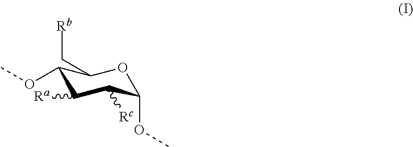

wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}—[F¹]ₚ-[L¹]_{q}—[F²]ᵣ-[L²]ᵥ-Z¹, and wherein R^{w}, R^{x}, and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4, and wherein at least one of R^{a}, R^{b} and R^{b} is —[O—(CR^{w}R^{x})—(CR^{y}R^{z})]_{y}—[F¹]ₚ-[L¹]_{q}—[F²]ᵣ-[L²]ᵥ-Z¹, wherein F¹ is selected from the group consisting of —O—, —S—, —NR^{Y7}— and —O—(C═Y⁶)—, wherein Y⁶ is selected from the group consisting of NR^{Y6}, O and S, more preferably Y⁶ is O, and wherein R^{Y6} is H or alkyl, preferably H, and wherein R^{Y7} is H or alkyl, preferably H, more preferably wherein F¹ is —O— or —O—(C═O)—, p is 0 or 1, and wherein L¹ is a linking moiety, preferably an alkyl, alkenyl, alkylaryl, arylalkyl, aryl, heteroaryl, alkylheteroaryl or heteroarylalkyl group, q is 0 or 1, with the proviso that in case p is 0, q is 0, F² is a functional group selected from the group consisting of —S—, —NH— and —NH—NH-T′-, wherein T′ is selected from the group consisting of —C(=T)-Y^{T}—, —SO₂—, aryl, and heteroaryl, wherein T is O or S, and wherein Y^{T} is —CH₂—, —O—, —NH— or —NH—NH—, preferably —O—, —NH— or —NH—NH—, r is 0 or 1, L² is a linking moiety, preferably an alkyl, alkenyl, alkylaryl, arylalkyl, aryl, heteroaryl, alkylheteroaryl or heteroarylalkyl group, v is 0 or 1, and wherein HAS″ is a remainder of HAS′,
and wherein step (a) comprises
(a1) providing a hydroxyalkyl starch having a mean molecular weight MW greater than or equal to 60 kDa and a molar substitution MS in the range of from 0.6 to 1.5 comprising the structural unit according to the following formula (II)

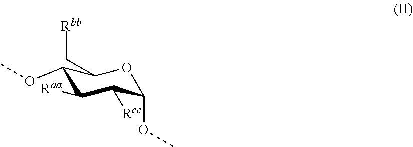

wherein R^{aa}, R^{bb} and R^{cc} are, independently of each other, selected from the group consisting of —O-HAS″ and —[O—(CR^{w}R^{x})—(CR^{w}R^{z})]ₓ—OH, and wherein R^{w}, R^{x}, R^{y} and R^{z} are independently of each other selected from the group consisting of hydrogen and alkyl, and wherein x is an integer in the range of from 0 to 20, preferably in the range of from 0 to 4,
(a2) introducing at least one functional group Z¹ into HAS by
   (i) coupling the hydroxyalkyl starch via at least one hydroxyl group comprised in HAS to at least one suitable linker comprising the functional group Z¹ or a precursor of the functional group Z¹, or
   (ii) displacing at least one hydroxyl group comprised in HAS in a substitution reaction with a suitable linker comprising the functional group Z¹ or a precursor thereof.

Furthermore, the present invention relates to a conjugate obtained or obtainable by said method.

According to a preferred embodiment of the present invention, the present invention relates to a method for preparing a hydroxyalkyl starch conjugate, as described above, as well as to a conjugate obtained or obtainable by said method, wherein the hydroxyalkyl starch derivative provided in step (a2) comprises at least one structural unit according to the following formula (I)

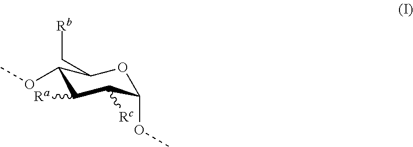

wherein R^{a}, R^{b} and R^{c} are independently of each other selected from the group consisting of —O-HAS″, —[O—CH₂—CH₂]ₛ—OH and —[O—CH₂—CH₂]ₜ—[F¹]ₚ-[L¹]_{q}—[F²]ᵥ-[L²]ᵥ-Z¹, and wherein at least one of R^{a}, R^{b} and R^{c} is —[O—CH₂—CH₂]ₜ—[F¹]ₚ-[L¹]_{q}—[F²]ᵣ-[L²]ᵥ-Z¹, and s is in the range of from 0 to 4, and t is in the range of from 0 to 4.
Step (a2)(i)

In step (a2)(i), the functional group Z¹ is introduced by coupling the hydroxyalkyl starch via at least one hydroxyl group to at least one suitable linker comprising the functional group Z¹ or a precursor of the functional group Z¹.

Organic chemistry offers a wide range of reactions to modify hydroxyl groups with linker constructs bearing functionalities such as the functional group Z¹, as described above. However, the hydroxyalkyl starch's polymeric nature and the abundance of hydroxyl groups present in the hydroxyalkyl starch usually strongly promote the number of possible side reactions such as inter- and intramolecular crosslinking. Therefore, a method was needed to functionalize the polymer under maximum retention of its molecular characteristics such as solubility, molecular weight and polydispersity. It was surprisingly found that when using the method according to the invention, possible side reactions such as inter- and intramolecular crosslinking can be significantly diminished.

According to a preferred embodiment of the present invention, in step (a2)(i), the hydroxyalkyl starch is coupled
to a suitable linker comprising the functional group Z¹ or a precursor thereof, preferably, the functional group Z¹ or the group —Z¹*—PG, with PG being a suitable protecting group,
   and Z¹* being the protected form of the functional group Z¹,
   said linker being capable of being coupled to a hydroxyl group of the hydroxyalkyl starch via Z², thereby forming a covalent linkage between the linker and the hydroxyalkyl starch, or
to a first linker, the first linker comprising a functional group K² and a precursor W of the functional group Z¹, said linker being capable of being coupled to a hydroxyl
   group of the hydroxyalkyl starch via K², thereby forming a covalent linkage between the first linker and the hydroxyalkyl starch, and wherein the precursor is
   transformed in at least one further step to give the functional group Z¹.

The precursor W is a functional group capable of being transformed to the functional group Z¹.

According to a first preferred embodiment of the invention, prior to the reaction with the at least one suitable linker, at least one hydroxyl group present in the hydroxyalkyl starch is initially activated with a reactive carbonyl compound, thereby generating a hydroxyalkyl starch derivative comprising a leaving group. The term “leaving group” as used in this context of the present invention is denoted to mean a molecular fragment that departs with a pair of electrons in heterolytic bond cleavage upon reaction with the at least one suitable linker, thereby forming a covalent bond between the atom formerly bearing the leaving group comprised in the activated hydroxyalkyl starch. Thus, in this case Z² is a nucleophilic group or Z² and Z¹ form together such a nucleophilic group capable of reacting with the activated hydroxyalkyl starch in the above-mentioned way.

The term “reactive carbonyl compound” as used in this context of the present invention, refers to carbonyl dication synthons having a structure R**—(C═O)—R*, wherein R* and R** may be the same or different, and wherein R* and R** are both leaving groups. As leaving groups halides, such as chloride, and/or residues derived from alcohols, may be used. The term “residue derived from alcohols”, refers to R* and/or R** being a unit —O—R^{ff} or —O—R^{gg}, with —O—R^{ff} and —O—R^{gg} preferably being residues derived from alcohols such as N-hydroxy succinimide or sulfo-N-hydroxy succinimide, suitably substituted phenols such as p-nitrophenol, o,p-dinitrophenol, o,o′-dinitrophenol, trichlorophenol such as 2,4,6-trichlorophenol or 2,4,5-trichlorophenol, trifluorophenol such as 2,4,6-trifluorophenol or 2,4,5-trifluorophenol, pentachlorophenol, pentafluorophenol, heterocycles such as imidazol or hydroxyazoles such as hydroxy benzotriazole may be mentioned. Further residues derived from alcohols having the structure alkyl-OH, such as methanol or ethanol may be mentioned. Reactive carbonyl compounds containing halides are phosgene, related compounds such as diphosgene or triphosgene, chloroformic esters and other phosgene substitutes known in the art.

Preferabyl, the reactive carbonyl compound having the structure R**—(C═O)—R* is selected from the group consisting of phosgene and the like (diphosgene, triphosgene), chloroformates such as p-nitrophenylchloroformate, pentafluorophenylchloroformate, phenylchloroformate, methyl- and ethylchloroformate, carbonic acid esters such as N,N′-disuccinimidyl carbonate, sulfo-N,N′-disuccinimidyl carbonate, dibenzotriazol-1-yl carbonate and carbonyldiimidazol. Especially preferred are carbonyldiimidazol (CDI), N,N′-disuccinimidyl carbonate, sulfo-N,N′-disuccinimidyl carbonate and p-nitrophenyl chloroformate.

Preferably upon reaction of at least one hydroxyl group with the reactive carbonyl compound R**—(C═O)—R* prior to the coupling step according to step (a2)(ii) an activated hydroxyalkyl starch derivative is formed, which, comprises at least one structural unit, preferably 3 to 200 structural units, more preferably 3 to 100 structural units, according to the following formula (Ib)

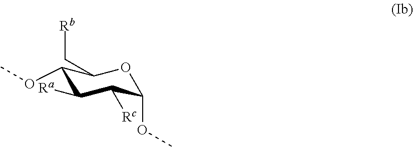

wherein R^{a}, R^{b} and R^{c} are independently of each other selected from the group consisting of —O-HAS″, —[O—CH₂—CH₂]ₛ—OH and —[O—CH₂—CH₂]ₜ—O—C(═O)—R*, wherein t is in the range of from 0 to 4, and wherein s is in the range of from 0 to 4, and wherein at least one of R^{a}, R^{b} and R^{c} comprises the group —[O—CH₂—CH₂]ₜ—O—C(═O)—R*, wherein R* is a leaving group, preferably a group selected from the group consisting of p-nitrophenoxy-, 2,4-dichlorophenoxy, 2,4,6-trichlorophenoxy, trichloromethoxy, imidazolyl, azides and halides, such as chloride or bromide.

In an alternative embodiment, R* is a group having the structure —O-alkyl, most preferably R* is —O—CH₃.

Thus, the present invention also relates to a method, as described above, wherein step (a2)(i) comprises
(aa) activating at least one hydroxyl group comprised in the hydroxyalkyl starch with a reactive carbonyl compound having the structure R^{d}**—(C═O)R*, wherein R* and R** may be the same or different, and wherein R* and R** are both leaving groups, wherein upon activation a hydroxyalkyl starch derivative comprising at least one structural unit according to the following formula (Ib)

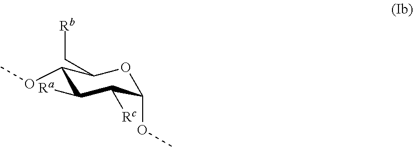

is formed, wherein R^{a}, R^{b} and R^{c} are, independently of each other, selected from the group consisting of —O-HAS″, —[O—CH₂—CH₂]ₛ—OH, and —[O—CH₂—CH₂]ₜ—O—C(═O)—R*, wherein s is in the range of from 0 to 4, and wherein t is in the range of from 0 to 4, wherein at least one of R^{a}, R^{b} and R^{c} comprises the group —[O—CH₂—CH₂]ₜ—O—C(═O)—R*, and
(bb) reacting the activated hydroxyalkyl starch according to step (aa) with at least one suitable linker comprising the functional group Z¹ or a precursor of the functional group Z¹, preferably reacting the activated hydroxyalkyl starch with a suitable linker comprising the functional group Z¹ and the functional group Z².

The invention further relates to a conjugate obtained or obtainable by said method.

In step (bb), the activated hydroxyalkyl starch derivative is preferably reacted with a linker having the structure Z²-[L²]ᵥ-Z¹, wherein v is 0 or I.

In case v is 1, Z² is preferably selected from the group consisting of HS—, H₂N— and H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or —NH—NH—, preferably —O—, —NH— or —NH—NH—. Thus, the following structures are preferred:

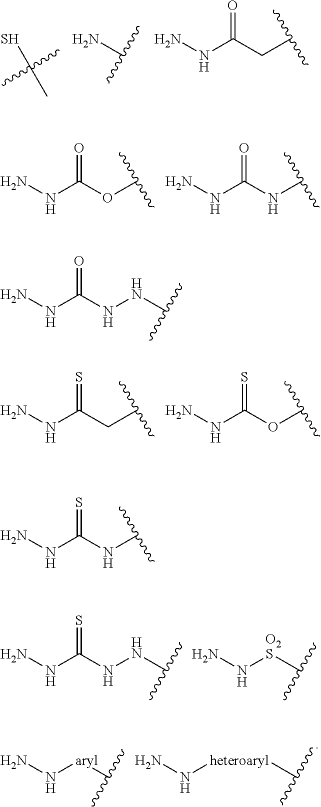

Preferably, Z² is H₂N—NH-T′-.

Thus, the present invention also relates to a method, as described above, wherein the activated hydroxyalkyl starch according to step (aa) is reacted with a linker having the structure Z²-[L²]ᵥ-Z¹, wherein v is 1 and wherein Z² has a structure according to the formula H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH—or —NH—NH—, preferably —O—, —NH— or —NH—NH—. Upon reaction of Z²-[L²]ᵥ-Z¹ with the group —O—C(═O)—R* comprised in the hydroxyalkyl starch derivative, the structural unit —[F¹]ₚ-[L¹]_{q}—[F²]ᵣ-[L²]ᵥ-Z¹ is formed, with q being 1, with F¹ being —O—C(═O)— and with F² being —NH—NH-T′-.

Most preferably, a symmetrical linker is used. By way of example, the following preferred linker compounds are mentioned:

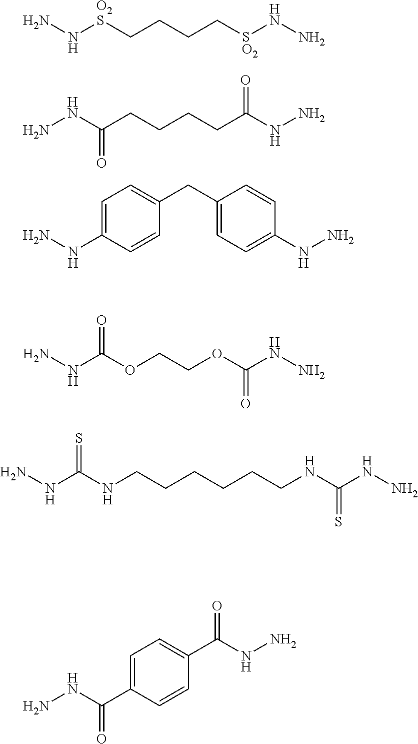

Alternatively, Z² is —SH. In this case, however, the linker preferably has the structure Z²-[L²]ᵥ-Z¹* —PG, with PG being a suitable protecting group such as tert-butyloxycarbonyl (BOC), 9-fluorenmethyloxycarbonyl (Fmoc) or benzyloxycarbonyl (Cbz) and Z¹* being the protected form of the functional group Z¹.

In case the linker comprises a protecting group, the method further comprises a deprotection step prior to step (b).

According to an alternative embodiment, v is 0 and the linker has the structure Z¹-Z². In this case, Z² is preferably H, H₂N—NH-aryl- or H₂N—NH-heteroaryl-. In case Z² is H₂N—NH-aryl-, Z¹ is preferably -aryl-NH—NH₂ giving a symmetrical linker Z²-aryl-aryl-Z¹, wherein the group aryl-aryl may also encompass monocyclic aromatic rings such as a benzene ring being substituted with two H₂N—NH— groups. In case Z² is H₂N—NH-heteroaryl-, Z¹ is preferably heteroaryl-NH—NH₂ giving a symmetrical linker Z²-heteroaryl-heteroaryl-Z¹, wherein the group heteroaryl-heteroaryl may also encompass monocyclic heteroaromatic rings being substituted with two H₂N—NH— groups.

In case v is 0, Z² is preferably H and Z¹ has the structure

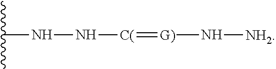

Upon reaction of Z²-[L²]ᵥ-Z¹ with v being 0, i.e. of Z²-Z¹, with the group —O—C(═O)—R^{*} comprised in the hydroxyalkyl starch derivative, the structural unit —[F¹]ₚ-[L¹]_{q}—[F²]ᵣ-[L²]ᵥ-Z¹ is formed, with q, r and v being 0.

Thus, the present invention also relates to a method, as described above, wherein in step (bb), the activated hydroxyalkyl starch derivative is reacted with a linker having the structure Z²-[L²]ᵥ-Z¹, wherein
v is 1, and Z² has a structure according to the formula H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, or —NH—NH—, preferably —O—, —NH— or —NH—NH—,
   and wherein upon reaction of Z²-[L²]ᵥ-Z¹ with the group —O—C(═O)—R* comprised in the hydroxyalkyl starch derivative, the structural unit —[F¹]ₚ-[L¹]_{q}—[F²]ᵣ-[L²]ᵥ-Z¹ is formed, with q being 1, and with F¹ being —O—C(═O)— and with F² being —NH—NH-T′-, or wherein
v is 0, Z² is H, and Z¹ has the structure

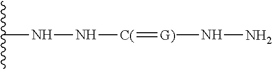

and wherein upon reaction of Z²-[L²]ᵥ-Z¹ with the group —O—C(═O)—R* comprised in the hydroxyalkyl starch derivative, the structural unit —[F¹]ₚ-[L¹]_{q}—[F²]ᵣ-[L²]ᵥ-Z¹ is formed, with q, r and v being 0.

Further, the present invention also relates to a conjugate obtained or obtainable by said method.

In the coupling reaction between the activated hydroxyalkyl starch and the linker, comprising the functional Z¹, in principle any reaction conditions known to those skilled in the art can be used. Preferably, the reaction is carried out in water, but mixtures with an organic solvent, such as N-methylpyrrolidone, dimethyl acetamide (DMA), dimethyl formamide (DMF), formamide, dimethyl sulfoxide (DMSO), or mixtures of two or more thereof are also possible. Alternatively, the reaction can be carried out without presence of water in organic solvents such as N-methylpyrrolidone, dimethyl acetamide (DMA), dimethyl formamide (DMF), formamide, dimethyl sulfoxide (DMSO), or mixtures of two or more thereof.

The reaction temperature is suitable chosen, depending on the leaving group R* present in the hydroxyalkyl starch derivative and depending on the functional group Z². Preferably, the reaction is carried out at a temperature in the range of from 5 to 100° C., more preferably in the range of from 5 to 50° C. and especially preferably in the range of from 15 to 30° C. The temperature may be held essentially constant or may be varied during the reaction procedure.

In an alternative embodiment, the reaction is carried out at a temperature in the range of from 30 to 100° C., preferably in the range of from 50 to 90° C., most preferably in the range of from 75 to 85° C. These higher temperature ranges are preferably employed in case R* is —O-alkyl, such as —O—CH₃. The temperature may be held essentially constant or may be varied during the reaction procedure.

The pH value for this reaction may be adapted to the specific needs of the reactants. Preferably, the pH value is greater than 7, preferably in the range of from 7 to 14, more preferably in the range of from 7.5 to 11.

The reaction may be carried out in the presence of a base. In particular, in case the reaction is carried out in at least one organic solvent comprising essentially no water, preferably comprising no water, at least one base is employed. Suitable bases are, for example, pyridine, substituted pyridines, such as 4-(dimethylamino)-pyridine, 2,6-lutidine or collidine, primary amine bases such as triethyl amine, diisopropyl ethyl amine (DIEA), N-methyl morpholine, amidine bases such as 1,8-diazabicyclo[5.4.0]undec-7-ene or inorganic bases such as alkali metal carbonates.

The reaction time for the reaction of activated hydroxyalkyl starch with the linker, preferably with the linker Z²-[L²]ᵥ-Z¹ may be adapted to the specific need and is generally in the range of from 1 h to 7 days, preferably 2 hours to 48 hours, more preferably 4 hours to 24 hours.

The derivative obtained according to step (a2)(i) comprising the functional group Z¹, may be subjected to at least one further isolation and/or purification step. According to a preferred embodiment of the present invention, the polymer derivative is first separated from the reaction mixture by a suitable method such as precipitation and subsequent centrifugation or filtration. In a second step, the separated polymer derivative may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first precipitated, subjected to centrifugation, redissolved and finally subjected to ultrafiltration.

Preferably, the precipitation is carried out with an organic solvent such as ethanol, isopropanol, acetone or tetrahydrofurane (THF). The precipitated conjugate is subsequently subjected to centrifugation and subsequent ultrafiltration using water or an aqueous buffer solution having a concentration preferably from 1 to 1000 mmol/l, more preferably from 1 to 100 mmol/l, and more preferably from 10 to 50 mmol/l such as about 20 mmol/l, a pH value in the range of preferably from 3 to 10, more preferably from 4 to 8, such as about 7. The number of exchange cycles preferably is from 5 to 50, more preferably from 10 to 30, and even more preferably from 15 to 25, such as about 20.

Most preferably the obtained derivative is further lyophilized until the solvent content of the reaction product is sufficiently low according to the desired specifications of the product.

In case the linker comprises a protecting group (PG), as described above, the method preferably further comprises a deprotection step. The reaction conditions used are adapted to the respective protecting group used.

### The Epoxide Modified Hydroxyalkyl Starch Derivative

According to a second preferred embodiment, in step (a2)(i), the hydroxyalkyl starch is initially coupled to a first linker, the first linker comprising a functional group K² and the functional group W, with W being a precursor of the functional group Z¹, said linker being capable of being coupled to a hydroxyl group of the hydroxyalkyl starch via K², thereby forming a covalent linkage between the first linker and the hydroxyalkyl starch. Preferably, the first linker comprises the functional group W, wherein W is an epoxide or a functional group which is transformed in a further step to give an epoxide.

Accordingly, the present invention also relates to a method for producing a hydroxyalkyl starch conjugate, as described above, wherein step (a2)(i) comprises step (I)
(I) coupling the hydroxyalkyl starch via at least one hydroxyl group comprised in the hydroxyalkyl starch to a first linker comprising a functional group W, wherein the functional group W is an epoxide or a group which is transformed in a further step to give an epoxide.

According to this second preferred embodiment, the linker comprises the functional group K², wherein K² is preferably a leaving group, preferably a leaving group being attached to a CH₂-group comprised in the at least one suitable linker which is reacted in step (a2)(ii) with the hydroxyalkyl starch. The term “leaving group” as used in this context of the present invention is denoted to mean a molecular fragment that departs with a pair of electrons in heterolytic bond cleavage upon reaction with the hydroxyl group of the hydroxyalkyl starch, thereby forming a covalent bond between the oxygen atom of the hydroxyl group and the carbon atom formerly bearing the leaving group. Common leaving groups are, for example, halides such as chloride, bromide and iodide, and sulfonates such as tosylates, mesylates, fluorosulfonates, triflates and the like. According to a preferred embodiment of the present invention, the functional group K² is a halide leaving group. Thus, upon reaction of the hydroxyl group with the functional group K², preferably the functional group F¹ is formed, which is preferably an —O— group.

Alternatively, K² may also be an epoxide group, which reacts with a hydroxyl group of HAS in a ring opening reaction, thereby forming a covalent bond.

Preferably, the first linker has the structure K²-L^{W}-W, wherein K² is a functional group capable of being reacted with at least one hydroxyl group of hydroxyalkyl starch, as described above, and wherein L^{W} is a linking moiety.

Thus, the present invention also relates to a method for preparing a hydroxyalkyl starch conjugate, as described above, and a hydroxyalkyl starch conjugate obtained or obtainable by said method, wherein step (a2)(i) comprises step (I)
(I) coupling the hydroxyalkyl starch via at least one hydroxyl group comprised in HAS to a first linker having a structure according to the following formula K²-L_{W}-W, wherein K² is a functional group capable of being reacted with at least one hydroxyl group of hydroxyalkyl starch, as described above, and wherein L^{W} is a linking moiety, and wherein, upon reaction of the hydroxyalkyl starch, a hydroxyalkyl starch derivative is formed comprising at least one structural unit, preferably 3 to 200 structural units, more preferably 3 to 100 structural units, according to the following formula (Ib)

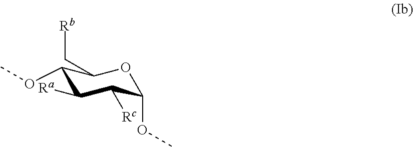

wherein R^{a}, R^{b} and R^{c} are, independently of each other, selected from the group consisting of —O-HAS″, —[O—(CR^{w}R^{x})—(CR_{y}R^{z})]ₓ—OH and —[O—(CR^{w}R^{x})—(CR^{y}R^{z})]_{y}—[F¹]ₚ-L^{W}-W, wherein R^{w}, R^{x}, R^{y} and R^{z} are independently of each other selected from the group consisting of hydrogen and alkyl, y is an integer in the range of from 0 to 20, preferably in the range of from 0 to 4, x is an integer in the range of from 0 to 20, preferably in the range of from 0 to 4 and wherein at least one of R^{a}, R^{b} and R^{c} comprises the group —[O—(CR^{w}R^{x})—CR^{y}R^{z})]_{y}—[F¹]ₚ-L^{W}-W, and wherein [F¹]ₚ, in this case, is the functional group being formed upon reaction of K² with the at least one hydroxyl group of the hydroxyalkyl starch, more preferably, wherein R^{a}, R^{b} and R^{c} are independently of each other selected from the group consisting of —O-HAS″, —[O—CH₂—CH₂]ₛ—OH and —[O—CH₂—CH₂]ₜ[F¹]ₚ-L^{W}-W, and wherein t is in the range of from 0 to 4 and wherein s is in the range of from 0 to 4, and p is 1, and wherein at least one of R^{a}, R^{b} and R^{c} comprises the group —[O—CH₂—CH₂]ₜ—[F¹]ₚ-L^{W}-W, and wherein [F¹]ₚ is the functional group being formed upon reaction of K² with the at least one hydroxyl group of the hydroxyalkyl starch.

According to one embodiment of the present invention, the functionalization of at least one hydroxyl group of hydroxyalkyl starch to give the epoxide comprising hydroxyalkyl starch, is carried out in a one-step procedure, wherein at least one hydroxyl group is reacted with a first linker, as described above, wherein the first linker comprises the functional group W, and wherein W is an epoxide.

Therefore, the present invention also relates to a method for preparing a hydroxyalkyl starch conjugate, as described above, as well as to a hydroxyalkyl starch conjugate obtained or obtainable by said method, wherein in step (a2)(i)(I) the hydroxyalkyl starch is reacted with a first linker comprising a functional group K² capable of being reacted with a hydroxyl group of the hydroxyalkyl starch, thereby forming a covalent linkage, the linker further comprising a functional group W, wherein the functional group W is an epoxide.

This linker has in this case a structure according to the following formula

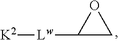

such as, for example, epichlorohydrine.

Upon reaction of this linker with at least one hydroxyl group of hydroxyalkyl starch, a hydroxyalkyl starch derivative is formed comprising at least one structural unit, preferably 3 to 200 structural units, more preferably 3 to 100 structural units, according to the following formula (Ib)

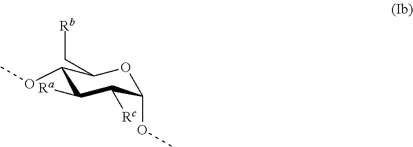

wherein R^{a}, R^{b} and R^{c} are independently of each other selected from the group consisting of —O-HAS″, —[O—(CR^{w}R^{x})—(CR^{y}R^{z})]ₓ—OH and and

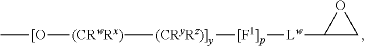

and wherein at least one of R^{a}, R^{b} and R^{c} comprises the group

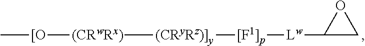

preferably wherein R^{a}, R^{b} and R^{c} are independently of each other selected from the group consisting of —O-HAS″, —[O—CH₂—CH₂]ₛ—OH and

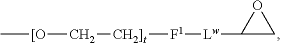

and wherein t is in the range of from 0 to 4 and wherein s is in the range of from 0 to 4, and p is 1, and wherein at least one of R^{a}, R^{b} and R^{c} comprises the group

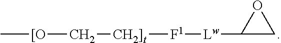

According to a preferred embodiment of the invention, the epoxide is generated in a two step procedure, comprising the steps (I) and (II)
(I) coupling at least one hydroxyl group of the hydroxyalkyl starch, preferably of hydroxyethyl starch, to a first linker, comprising a functional group K² capable of being reacted with a hydroxyl group of the hydroxyalkyl starch, thereby forming a covalent linkage between the first linker and the hydroxyalkyl starch, the linker further comprising a functional group W, wherein the functional group W is a functional group which is capable of being transformed in a further step to give an epoxide, such as an alkenyl group,
(II) transforming the functional group W to give an epoxide.

It was surprisingly found that this two step procedure is superior to the one step procedure in that higher loadings of the hydroxyalkyl starch with epoxide groups can be achieved and/or undesired side reactions such as inter- and intra-molecular crosslinking can be substantially avoided.

Preferably, the functional group W is an alkenyl group. In this case, step (II) preferably comprises the oxidation of the alkenyl group to give an epoxide and transforming the epoxide to give the functional group Z¹.

Therefore, the present invention also describes a method for preparing a hydroxyalkyl starch conjugate, as described above, as well as to a hydroxyalkyl starch conjugate obtained or obtainable by said method wherein W is an alkenyl group and the method further comprises
(II) oxidizing the alkenyl group W to give the epoxide,
   and wherein in step (II), preferably a hydroxyalkyl starch derivative comprising at least one structural unit according to the following formula (I) is formed

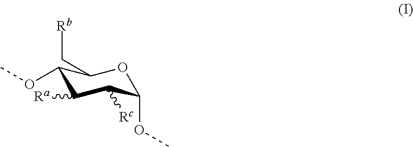

wherein R^{a}, R^{b} and R^{c} are independently of each other, selected from the group consisting of —O-HAS″, —[O—CH₂—CH₂]ₛ—OH and

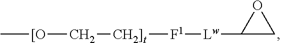

and wherein at least one of R^{a}, R^{b} and R^{c} is

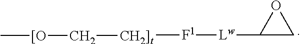

According to the present invention, the term “linking moiety L^{W}” as used in the context of the present invention relates to any suitable chemical moiety bridging the functional group K² and the functional group W.

In general, there are no particular restrictions as to the chemical nature of the linking moiety L^{W} with the proviso that L^{W} has particular chemical properties enabling carrying out the inventive method for the preparation of the novel derivatives comprising the functional group Z¹, i.e. in particular, in case W is a functional group to be transformed to an epoxide, the linking moiety L^{W} has suitable chemical properties enabling the transformation of the chemical moiety W to the functional group Z¹. According to a preferred embodiment of the present invention, L^{W} bridging W and HAS′ comprises at least one structural unit according to the following formula

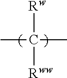

wherein R^{vv} and R_{ww} are, independently from each other, H or an organic residue selected from the group consisting of alkyl, alkenyl, alkylaryl, arylalkyl, aryl, heteroaryl, alkylheteroaryl and heteroarylalkyl groups.

Preferably, L^{W} is an optionally substituted, non-branched alkyl residue such as a group selected from the following groups:

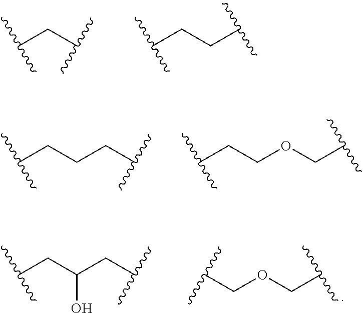

According to a first preferred embodiment of the present invention, the functional group W is an alkenyl group, wherein the first linker K²-L^{W}-W has a structure according to the following formula

K²-L^{W}-CH═CH₂

preferably with K² being a leaving group or an epoxide.

Thus, preferred structures of the first linker are by way of example, the following structures:

Hal-CH₂—CH═CH₂, such as Cl—CH₂—CH═CH₂ or Br—CH₂—CH═CH₂ or I—CH₂—CH═CH₂ sulfonic esters such as TsO—CH₂—CH═CH₂ or MsO—CH₂—CH═CH₂ epoxides such as

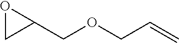

More preferably, with K² in the first linker K²-L^{W}-W being a leaving group, most preferably the first linker K²-L^{W}-W has a structure according to the following formula

Hal-L_{W}-CH═CH₂.

According to an especially preferred embodiment of the present invention, the linker K²-L^{W}-W has a structure according to the following formula

Hal-CH₂—CH═CH₂

with Hal being a halogen, preferably the halogen being I, Cl, or Br, more preferably Br.

Thus, the present invention also relates to a method for preparing a hydroxyalkyl starch conjugate, as described above, wherein in step (a2)(ii) the hydroxyalkyl starch, preferably the hydroxyethyl starch, is coupled via at least one hydroxyl group to at least one suitable linker having the structure Hal-CH₂—CH═CH₂, wherein upon reaction of the hydroxyalkyl starch with the linker, a hydroxyalkyl starch derivative is formed comprising at least one structural unit according to the following formula (Ib)

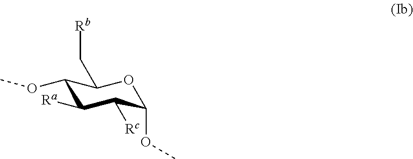

wherein R^{a}, R^{b} and R^{c} are independently of each other selected from the group consisting of —O-HAS″, —[O—(CR^{w}R^{x})—(CR^{y}R^{z})]ₓ—OH and —[O—(CR^{w}R^{x})—(CR^{y}R^{z})]_{y}—O—CH₂—CH═CH₂, and wherein at least one of R^{a}, R^{b} and R^{c} comprises the group —[O—(CR^{w}R^{x})—(CR^{y}R^{z})]_{y}—O—CH₂—CH═CH₂, preferably wherein R^{a}, R^{b} and R^{c} are independently of each other selected form the group consisting of —OH, —O-HAS″, [O—CH₂—CH₂]ₛ—OH and —[O—CH₂—CH₂]ₜ—O—CH₂—CH═CH₂, wherein t is in the range of from 0 to 4, wherein s is in the range of from 0 to 4, and wherein at least one of R^{a}, R^{b} and R^{c} comprises the group —[O—CH₂—CH₂]ₜ—O—CH₂—CH═CH₂, and wherein the functional group —O— linking the —CH₂—CH═CH₂ group to the hydroxyalkyl starch is formed upon reaction of the linker Hal-CH₂—CH═CH₂ with the hydroxyl group of the hydroxyalkyl starch. Likewise, the present invention also relates to a hydroxyalkyl starch conjugate obtained or obtainable by the above-mentioned method.

As regards the reaction conditions used in this step (I), wherein the hydroxyalkyl starch is reacted with the first linker, in particular wherein the first linker comprises the functional group W with W being an alkenyl, in principle any reaction conditions known to those skilled in the art can be used. Preferably, the reaction is carried out in an organic solvent, such as N-methylpyrrolidone, dimethyl acetamide (DMA), dimethyl formamide (DMF), formamide, dimethyl sulfoxide (DMSO) or mixtures of two or more thereof. More preferably, the reaction is carried out in anhydrous solvents or solvent mixtures.

Preferably, the hydroxyalkyl starch is dried prior to use, by means of heating to constant weight at a temperature range from 50 to 80° C. in a drying oven or with related techniques.

The temperature of the reaction is preferably in the range of from 5 to 55° C., more preferably in the range of from 10 to 30° C., and especially preferably in the range of from 15 to 25° C. During the course of the reaction, the temperature may be varied, preferably in the above given ranges, or held essentially constant.

The reaction time for the reaction of HAS with the linker K²-L^{W}-W may be adapted to the specific needs and is generally in the range of from 1 h to 7 days, preferably 2 hours to 24 hours, more preferably 3 hours to 18 hours.

More preferably, the reaction is carried out in the presence of a base. The base may be added together with the linker K²-L^{W}-W, or may be added prior to the addition of the linker, to pre-activate the hydroxyl groups of the hydroxyalkyl starch. Preferably, a base, such as alkali metal hydrides, alkali metal hydroxides, alkali metal carbonates, amine bases such as diisopropylethyl amine (DIEA) and the like, amidine bases such as 1,8-diazabicyclo[5.4.0]undec-7-ene (DBU), amide bases such as lithium diisopropylamide (LDA) or alkali metal hexamethyldisilazyl bases (e.g. LiHMDS) may be used. Most preferably the hydroxyalkyl starch is pre-activated with sodium hydride prior to the addition of the first linker K²-L^{W}-W.

The derivative comprising the functional group W, preferably the alkenyl group, may be isolated prior to transforming this group in at least one further step to give an epoxide comprising the hydroxyalkyl starch derivative. Isolation of this polymer derivative comprising the functional group W may be carried out by a suitable process which may comprise one or more steps. According to a preferred embodiment of the present invention, the polymer derivative is first separated from the reaction mixture by a suitable method such as precipitation and subsequent centrifugation or filtration. In a second step, the separated polymer derivative may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first precipitated, subjected to centrifugation, redissolved and finally subjected to ultrafiltration.

Preferably, the precipitation is carried out with an organic solvent such as ethanol, isopropanol, acetone or tetrahydrofurane (THF). The precipitated derivative is subsequently subjected to centrifugation and subsequent ultrafiltration using water or an aqueous buffer solution having a concentration preferably from 1 to 1000 mmol/l, more preferably from 1 to 100 mmol/l, and more preferably from 10 to 50 mmol/l such as about 20 mmol/I, a pH value in the range of preferably from 3 to 10, more preferably from 4 to 8, such as about 7. The number of exchange cycles preferably is from 5 to 50, more preferably from 10 to 30, and even more preferably from 15 to 25, such as about 20. Most preferably the obtained derivative comprising the functional group W is further lyophilized until the solvent content of the reaction product is sufficiently low according to the desired specifications of the product.

In case W is an alkenyl, the method preferably further comprises step (II), that is the oxidation of the alkenyl group to give an epoxide group. As to the reaction conditions used in the epoxidation (oxidation) step (II), in principle, any known method to those skilled in the art can be applied to oxidize an alkenyl group to yield an epoxide.

The following oxidizing reagents are mentioned, by way of example, metal peroxysulfates such as potassium peroxymonosulfate (Oxone®) or ammonium peroxydisulfate, peroxides such as hydrogen peroxide, tert.-butyl peroxide, acetone peroxide (dimethyldioxirane), sodium percarbonate, sodium perborate, peroxy acids such as peroxoacetic acid, meta-chloroperbenzoic acid (MCPBA) or salts like sodium hypochlorite or hypobromite.

According to a particularly preferred embodiment of the present invention, the epoxidation is carried out with potassium peroxymonosulfate (Oxone®) as oxidizing agent.

Thus, the present invention also relates to a method for preparing a hydroxyalkyl starch conjugate, as described above, wherein step (a2)(i) comprises
(I) coupling at least one hydroxyl group of the hydroxyalkyl starch, preferably of hydroxyethyl starch, to a first linker, comprising a functional group K² capable of being reacted with a hydroxyl group of the hydroxyalkyl starch, thereby forming a covalent linkage between the first linker and the hydroxyalkyl starch, the linker further comprising a functional group W, wherein the functional group W is an alkenyl group,
(II) oxidizing the alkenyl group to give an epoxide, wherein as oxidizing agent, preferably potassium peroxymonosulfate (Oxone®) is employed.

Further, the present invention also relates to a hydroxyalkyl starch conjugate obtained or obtainable by said method.

According to an even more preferred embodiment of the present invention, the reaction with potassium peroxymonosulfate (Oxone®) is carried out in the presence of a suitable catalyst. Catalysts may consist of transition metals and their complexes, such as manganese (Mn-salene complexes are known as Jacobsen catalysts), vanadium, molybdenium, titanium (Ti-dialkyltartrate complexes are known as Sharpless catalysts), rare earth metals and the like. Additionally, metal free systems can be used as catalysts. Acids such as acetic acid may form peracids in situ and epoxidize alkenes. The same accounts for ketones such as acetone or tetrahydrothiopyran-4-one, which react with peroxide donors under formation of dioxiranes, which are powerful epoxidation agents. In case of non-metal catalysts, traces of transition metals from solvents may lead to unwanted side reactions, which can be excluded by metal chelation with EDTA.

Preferably, said suitable catalyst is tetrahydrothiopyran-4-one.

Upon epoxidation, in step (II) a. hydroxyalkyl starch derivative is formed comprising at least one structural unit, preferably 3 to 200 structural units, more preferably 3 to 100 structural units, according to the following formula (Ib)

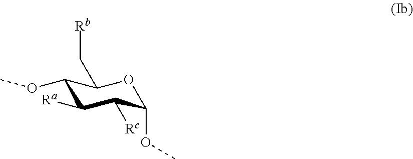

wherein R^{a}, R^{b} and R^{c} are independently of each other selected from the group consisting of —O-HAS″, —[O—(CR^{w}R^{x})—(CR^{y}R^{x})]ₓ—OH and and

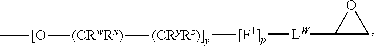

and wherein at least one of R^{a}, R^{b} and R^{c} comprises the group

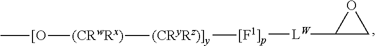

preferably wherein R^{a}, R^{b} and R^{c} are independently of each other selected from the group consisting of —O-HAS″, —[O—CH₂—CH₂]ₛ—OH and

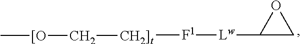

wherein t is in the range of from 0 to 4 and wherein s is in the range of from 0 to 4, and p is 1, and wherein at least one of R^{a}, R^{b} and R^{c} comprises the group

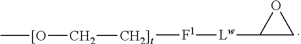

According to a preferred embodiment, the epoxidation of the alkenyl-modified hydroxyalkyl starch derivatives is carried out in aqueous medium, preferably at a temperature in the range of from 0 to 80° C., more preferably in the range of from 0 to 50° C. and especially preferably in the range of from 10 to 30° C.

During the course of the epoxidation reaction, the temperature may be varied, preferably in the above-given ranges, or held essentially constant. The term “aqueous medium” as used in the context of the present invention refers to a solvent or a mixture of solvents comprising water in an amount of at least 10% per weight, preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The aqueous medium may comprise additional solvents like formamide, dimethylformamide (DMF), dimethylsulfoxide (DMSO), alcohols such as methanol, ethanol or isopropanol, acetonitrile, tetrahydrofurane or dioxane. Preferably, the aqueous solution contains a transition metal chelator (disodium ethylenediaminetetraacetate, EDTA, or the like) in a concentration ranging from 0.01 to 100 mM, preferably 0.01 to 1 mM, most preferably 0.1 to 0.5 mM, such as about 0.4 mM.

The pH value for the reaction of the HAS derivative with potassium peroxymonosulfate (Oxone®) may be adapted to the specific needs of the reactants. Preferably, the reaction is carried out in buffered solution, at a pH value in the range of from 3 to 10, more preferably of from 5 to 9, and even more preferably of from 7 to 8. Among the preferred buffers, carbonate, phosphate, borate and acetate buffers as well as tris(hydroxymethyl)aminomethane (TRIS) may be mentioned. Among the preferred bases, alkali metal bicarbonates may be mentioned.

According to the invention, the epoxide-modified HAS derivative may be purified or isolated in a further step prior to the transformation of the epoxide group to the functional group Z¹.

The separated derivative is optionally lyophilized.

After the purification step, the HAS derivative is preferably obtained as a solid. According to a further conceivable embodiment of the present invention, the HAS derivative solutions or frozen HAS derivative solutions may be mentioned.

The epoxide comprising HAS derivative is preferably reacted in a subsequent step (III) with at least one suitable reagent to yield the HAS derivative comprising the functional group Z¹. Preferably, the epoxide is reacted with a further linker having the structure Z²-[L²]ᵥ-Z¹ or Z²-[L²]ᵥ-Z¹* —PG, wherein PG is a suitable protecting group, as described above, and wherein Z¹* i the protected form of the functional group Z¹.

Preferably, the linker Z²-[L²]ᵥ-Z¹ reacts with the epoxide in a ring opening reaction and yields a HAS derivative comprising at least one structural unit according to the following formula (Ib)

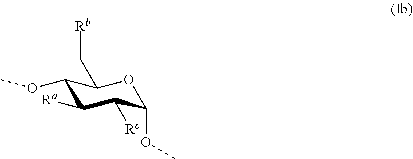

wherein at least one of R^{a}, R^{b} and R^{c} is —[O—(CR^{w}R^{x})—(CR^{y}R^{z})]_{y}—[F¹]ₚ-[L¹]_{q}—[F²]ᵣ-[L²]ᵥ-Z¹-.

Depending on integer v, the following different embodiments are preferred:
According to one preferred embodiment, v is 1. In this case, Z² has preferably a structure selected from the group consisting of HS—, H₂N— and H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or —NH—NH—, preferably —O—, —NH— or —NH—NH—. Preferred structures of the linker Z²-[L²]ᵥ-Z¹ are described hereinabove.
   Upon reaction the linker with the structural unit

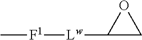

comprised in the hydroxyalkyl starch derivative, the structural unit —[F¹]ₚ-[L¹]_{q}—[F²]ᵣ-[L²]ᵥ-Z¹ is formed, with q being 1, and with p being 1 and with F¹ being —O—, and wherein r is 1 and F² is selected from the group consisting of —S—, —NH— and —NH—NH-T′, wherein L¹ preferably has the structure —CH₂—CHOH—CH₂— or —CH₂—CH(CH₂OH)—.
According to an alternative embodiment, v is 0. In this case Z² is preferably H, as described above, with Z¹ having the structure

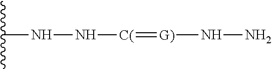

In this case upon reaction of Z² with the structural unit

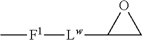

comprised in the hydroxyalkyl starch derivative, a structural unit —[F¹]ₚ-[L¹]_{q}—[F²]ᵣ-[L²]ᵥ-Z¹ is formed, with q being 1, and with r being 0 and with v being 0, wherein L¹ preferably has the structure —CH₂—CHOH—CH₂— or —CH₂—CH(CH₂OH)—.

In the reaction between the epoxide and the linker, comprising the functional Z¹, in principle any reaction conditions known to those skilled in the art can be used. Preferably, the reaction is carried out in water, but mixtures with organic solvents, such as N-methyl pyrrolidone, dimethyl acetamide (DMA), dimethyl formamide (DMF), formamide, dimethyl sulfoxide (DMSO), or mixtures of two or more thereof are possible. Preferably, the reaction is carried out at a temperature in the range of from 5 to 80° C., more preferably in the range of from 5 to 50° C. and especially preferably in the range of from 15 to 30° C. The temperature may be held essentially constant or may be varied during the reaction procedure.

The pH value for this reaction may be adapted to the specific needs of the reactants. The pH value is preferably greater than 7.

The reaction may be carried out in the presence of a base. In particular, in case the reaction is carried out in at least one organic solvent comprising essentially no water, preferably comprising no water, at least one base is employed. Suitable bases are, for example, pyridine, substituted pyridines, such as 4-(dimethylamino)-pyridine, 2,6-lutidine or collidine, primary amine bases such as triethyl amine, diisopropyl ethyl amine (DIEA), N-methyl morpholine, amidine bases such as 1,8-diazabicyclo[5.4.0]undec-7-ene or inorganic bases such as alkali metal carbonates may be mentioned.

The reaction time for the reaction of the epoxide comprising hydroxyalkyl starch with the linker, preferably with the linker Z²-[L²]ᵥ-Z¹ may be adapted to the specific needs and is generally in the range of from 1 h to 7 days, preferably 2 hours to 48 hours, more preferably 4 hours to 24 hours.

The derivative obtained according to step (a2)(i) comprising the functional group Z¹, may be subjected to at least one further isolation and/or purification step. According to a preferred embodiment of the present invention, the polymer derivative is first separated from the reaction mixture by a suitable method such as precipitation and subsequent centrifugation or filtration. In a second step, the separated polymer derivative may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first precipitated, subjected to centrifugation, redissolved and finally subjected to ultrafiltration.

Preferably, the precipitation is carried out with an organic solvent such as ethanol, isopropanol, acetone or tetrahydrofurane (THF). The precipitated conjugate is subsequently subjected to centrifugation and subsequent ultrafiltration using water or an aqueous buffer solution having a concentration preferably from 1 to 1000 mmol/l, more preferably from 1 to 100 mmol/l, and more preferably from 10 to 50 mmol/l such as about 20 mmol/l, a pH value in the range of preferably from 3 to 10, more preferably from 4 to 8, such as about 7. The number of exchange cycles preferably is from 5 to 50, more preferably from 10 to 30, and even more preferably from 15 to 25, such as about 20.

Most preferably the obtained derivative is further lyophilized until the solvent content of the reaction product is sufficiently low according to the desired specifications of the product.

Step (a2)(ii)

As regards step (a2)(ii) of the method according to the present invention, in this step, the functional group Z¹ is introduced by displacing a hydroxyl group present in the hydroxyalkyl starch in a substitution reaction with a linker comprising the functional group Z¹ or a precursor thereof.

Preferably, the suitable linker according to step (a2)(ii) has the structure Z²-[L²]ᵥ-Z¹, with Z², L² and Z¹ being as described above.

Preferably, prior to the replacement of the hydroxyl group with the functional group Z¹, the at least one hydroxyl group of the hydroxyalkyl starch is activated to generate a suitable leaving group. Preferably, a group R^{L} is added to the at least one hydroxyl group thereby generating a group —O—R^{L}, wherein the structural unit —O—R^{L} is the leaving group.

Thus, the present invention also relates to a method for preparing a hydroxyalkyl starch conjugate, as described above, as well as to a hydroxyalkyl starch conjugate obtained or obtainable by said method wherein in step (a2)(ii), prior to the substitution (displacement) of the hydroxyl group with the group comprising the functional group Z¹ or a precursor thereof, a group R^{L} is added to at least one hydroxyl group thereby generating a group —O—R^{L}, wherein —O—R¹ is the leaving group.

The term “leaving group” as used in this context of the present invention is denoted to mean that the molecular fragment —O—R^{L} departs when reacting the hydroxyalkyl starch derivative with the linker comprising the functional group Z¹ or a precursor thereof.

As regards preferred leaving groups used in this context of the present invention, according to a preferred embodiment, the hydroxyl group is transformed to a sulfonic ester, such as a mesylic ester (-OMs), tosylic ester (-OTs), imsyl ester (imidazylsulfonyl ester) or a carboxylic ester such as trifluoroacetic ester.

Preferably, the at least one leaving group is generated by reacting at least one hydroxyl group of hydroxyalkyl starch, preferably in the presence of a base, with the respective sulfonyl chloride to give the sulfonic ester, preferably the mesylic ester.

Thus, the present invention also relates to a method for preparing a hydroxyalkyl starch conjugate as described above, as well as to a hydroxyalkyl starch conjugate obtained or obtainable by said method, wherein in step (a2)(ii), prior to the substitution (displacement) of the hydroxyl group with the linker comprising the functional group Z¹ or a precursor thereof, a group R^{L} is added to at least one hydroxyl group, thereby generating a group —O—R^{L}, wherein —O—R^{L} is a leaving group, in particular a O-Mesyl (-OMs) or —O— Tosyl (-OTs) group.

The addition of the group R^{L} to at least one hydroxyl group of hydroxyalkyl starch, whereupon a group —O—R^{L} is formed, is preferably carried out in an organic solvent, such as N-methylpyrrolidone, dimethyl acetamide (DMA), dimethyl formamide (DMF), formamide, dimethylsulfoxide (DMSO) and mixtures of two or more thereof, preferably at a temperature in the range of from −60 to 80° C., more preferably in the range of from −30 to 50° C. and especially preferably in the range of from −30 to 30° C. The temperature may be held essentially constant or may be varied during the reaction procedure. The pH value for this reaction may be adapted to the specific needs of the reactants. Preferably, the reaction is carried out in the presence of a base. Among the preferred bases pyridine, substituted pyridines such as collidine or 2,6-lutidine, amine bases such as triethylamine, diisopropyl ethyl amine (DIEA), N-methylmorpholine, N-methylimidazole or amidine bases such as 1,8-diazabicyclo[5.4.0]undec-7-ene (DBU) and inorganic bases such as metal hydrides and carbonates may be mentioned. Especially preferred are substituted pyridines (collidine) and tertiary amine bases (DIEA, N-methylmorpholine). The reaction time for this reaction step may be adapted to the specific needs and is generally in the range of from 5 min to 24 hours, preferably 15 min to 10 hours, more preferably 30 min to 5 hours.

The derivative comprising the group —O—R^{L}, may be subjected to at least one further isolation and/or purification step such as precipitation and/or centrifugation and/or filtration prior to the substitution reaction according to step (a2)(ii). Likewise, instead or additionally, the derivative comprising the —O—R^{L} group may be subjected to an after-treatment like ultrafiltration, dialysis, centrifugal filtration or pressure filtration, ion exchange chromatography, reversed phase chromatography, HPLC, MPLC, gel filtration and/or lyophilisation. According to a preferred embodiment, the derivative comprising the —O—R^{L} is in situ reacted with the precursor of the functional group Z¹ or with the bifunctional linker, comprising the functional group Z¹ or a precursor thereof.

As described above, the at least one hydroxyl group, preferably the at least one —O—R^{L} group more preferably the -OMs group or the -OTs group, is subsequently displaced, in a substitution reaction.

As described above, the linker preferably has the structure Z²-[L²]ᵥ-Z¹.

According to one embodiment, v is 1 and Z² has a structure selected from the group consisting of HS—, H₂N— and H₂N—NH-T′-, more preferably a structure according to the formula H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or —NH—NH—, preferably —O—, —NH— or —NH—NH—.

Preferred linker structures are described hereinabove.

Upon this reaction the structural unit —[F¹]ₚ-[L¹]_{q}—[F²]ᵣ-[L²]ᵥ-Z¹ is formed, with p and q being 0, and with r being 1 and with F² being selected from the group consisting of —S—, —NH— and —NH—NH-T′, preferably, wherein F² is —NH—NH-T′-.

According to an alternative embodiment, v is 0. In this case Z² is preferably H, as described above, with Z¹ having the structure

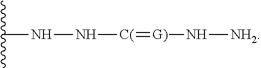

In this substitution step, in principle any reaction conditions known to those skilled in the art can be used. Preferably, the reaction is carried out in organic solvents, such as N-methyl pyrrolidone, dimethyl acetamide (DMA), dimethyl formamide (DMF), formamide, dimethyl sulfoxide (DMSO) and mixtures of two or more thereof. Preferably this step is carried out at a temperature in the range of from 0 to 80° C., more preferably in the range of from 20 to 70° C. and especially preferably in the range of from 40 to 60° C. The temperature may be held essentially constant or may be varied during the reaction procedure.

The pH value for this reaction may be adapted to the specific needs of the reactants. Optionally, the reaction followed by a capping reaction, thus removing residual active groups on the HAS. Preferably, the capping reagent is mercaptoethanol.

The reaction time for the substitution step is generally in the range of from 1 hour to 7 days, preferably 3 to 48 hours, more preferably 4 to 18 hours.

The derivative obtained may be subjected to at least one further isolation and/or purification step, as described above.

It has to be understood, that in case at least one hydroxyl group present in hydroxyalkyl starch, comprising the structural unit according to the following formula (II)

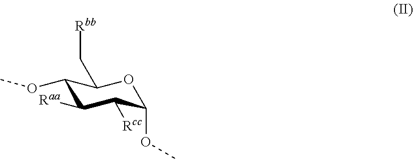

with R^{aa}, R^{bb} and R^{cc} being independently of each other selected from the group consisting of —[O—(CR^{w}R^{x})—(CR^{y}R^{z})]ₓ—OH and —O-HAS″, is displaced in a substitution reaction, the stereochemistry of the carbon atom which bears the respective hydroxyl function, which is displaced, may be inverted.

Thus, in case at least one of R^{aa} and R^{bb} in the above shown structural unit is —OH, and in case, this at least one group is displaced by a precursor of the functional group Z¹, thereby yielding a hydroxyalkyl starch derivative comprising the functional group Z¹ in this structural unit, the stereochemistry of the carbon atoms bearing this functional group Z¹ may be inverted.

Since, it cannot be excluded that such a substitution of primary hydroxyl groups occurs, in the method of the invention according to step (a2)(ii), the stereochemistry of the carbon atoms bearing the functional group R^{a} and R^{c} is not further defined, as shown in the structural unit according to the following formula (I)

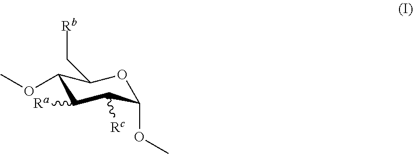

However, without wanting to be bound to any theory, it is believed that mainly primary hydroxyl groups will be displaced in the substitution reaction according to step (a2)(ii). Thus, according to this theory, the stereochemistry of most carbon atoms bearing the residues R^{a} or R^{c} will not be inverted but the respective structural unit of the hydroxyalkyl starch will comprise the stereochemistry as shown in the formula (Ib)

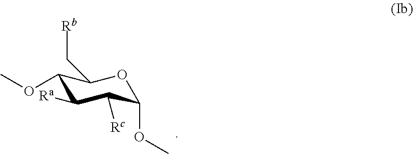

### Step (b)

As already described above, the hydroxyalkyl starch obtained according to step (a) is, optionally after at least one purification and/or isolation step, further reacted in step (b).

In step (b) the HAS derivative is coupled via the functional group Z¹ to at least one cytotoxic agent via a carbonyl function comprised in said cytotoxic agent.

As regards to the reaction conditions used in step (b), in principle any reaction conditions known to those skilled in the art can be used. Preferably, the reaction is carried out in an aqueous reaction medium, preferably in a mixture comprising water and at least one organic solvent, preferably at least one water miscible solvent, in particular a solvent selected from the group such as N-methylpyrrolidone, dimethyl acetamide (DMA), dimethyl formamide (DMF), formamide, dimethyl sulfoxide (DMSO), acetonitrile, tetrahydrofurane (TI-IF), dioxane, alcohols such as methanol, ethanol, isopropanol and mixtures of two or more thereof. More preferably, the reaction is carried out in aqueous buffer.

The temperature of the reaction is preferably in the range of from 5 to 55° C., more preferably in the range of from 10 to 30° C., and especially preferably in the range of from 15 to 25° C. During the course of the reaction, the temperature may be varied, preferably in the above given ranges, or held essentially constant.

The reaction time for the reaction of step (b) may be adapted to the specific needs and is generally in the range of from 30 min to 2 days, preferably 1 hour to 18 hours, more preferably 2 hours to 6 hours.

The pH value for the reaction of step (b) may be adapted to the specific needs of the reactants. Preferably, the reaction is carried in a buffered solution, at a pH value in the ranges of from 2 to 8, more preferably of from 3 to 7, most preferably of from 4 to 6. Among the preferred buffers, citrate buffer and acetate buffer shall be mentioned.

Preferably, the hydroxyalkyl starch conjugate obtained according to step (b) is subjected to at least one isolation and/or purification step. Isolation of the conjugate may be carried out by a suitable process which may comprise one or more steps.

According to a preferred embodiment of the present invention, the conjugate is first separated from the reaction mixture by a suitable method such as precipitation and subsequent centrifugation or filtration. In a second step, the separated conjugate may be subjected to a further treatment such as an after-treatment like ultrafiltration, dialysis, centrifugal filtration or pressure filtration, ion exchange chromatography, reversed phase chromatography, HPLC, MPLC, gel filtration and/or lyophilisation. According to an even more preferred embodiment, the separated polymer derivative is first precipitated, subjected to centrifugation, redissolved and finally subjected to ultrafiltration.

Preferably, the precipitation is carried out with an organic solvent such as ethanol or isopropanol. The precipitated conjugate is subsequently subjected to centrifugation and subsequent ultrafiltration using water or an aqueous buffer solution having a concentration preferably from 1 to 1000 mmol/l, more preferably from 1 to 100 mmol/l, and more preferably from 10 to 50 mmol/l such as about 20 mmol/l, a pH value in the range of preferably from 6 to 10, more preferably from 7 to 9, such as about 8. The number of exchange cycles preferably is from 5 to 50, more preferably from 10 to 30, and even more preferably from 15 to 25, such as about 20.

Most preferably, the obtained conjugate is further lyophilized until the solvent content of the reaction product is sufficiently low according to the desired specifications of the product.

### Pharmaceutical Composition

Furthermore, the present invention relates to a pharmaceutical composition comprising in a therapeutically effective amount a HAS conjugate, as described above, or a HAS conjugate obtained or obtainable by the above described method.

As far as the pharmaceutical compositions according to the present invention comprising the hydroxyalkyl starch conjugate, as described above, are concerned, the hydroxyalkyl starch conjugate may be used in combination with a pharmaceutical excipient. Generally, the hydroxyalkyl starch conjugate will be in a solid form which can be combined with a suitable pharmaceutical excipient that can be in either solid or liquid form. As excipients, carbohydrates, inorganic salts, antimicrobial agents, antioxidants, surfactants, buffers, acids, bases, and combinations thereof may be mentioned. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient may also include an inorganic salt or buffer such as citric acid, sodium chloride, potassium chloride, sodium sulfate, potassium nitrate, sodium phosphate monobasic, sodium phosphate dibasic, and combinations thereof. The pharmaceutical composition according to the present invention may also comprise an antimicrobial agent for preventing or determining microbial growth, such as, e.g., benzalkonium chloride, benzethonium chloride, benzyl alcohol, cetylpyridinium chloride, chlorobutanol, phenol, phenylethyl alcohol, phenylmercuric nitrate, thimersol, and combinations thereof.

The pharmaceutical composition according to the present invention may also comprise an antioxidant, such as, e.g., ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

The pharmaceutical composition according to the present invention may also comprise a surfactant, such as, e.g., polysorbates, or pluronics sorbitan esters; lipids, such as phospholipids and lecithin and other phosphatidylcholines, phosphatidylethanolamines, acids and fatty esters; steroids, such as cholesterol; and chelating agents, such as EDTA or zinc.

The pharmaceutical composition according to the present invention may also comprise acids or bases such as, e.g., hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof, and/or sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumarate, and combinations thereof. Generally, the excipient will be present in the pharmaceutical composition according to the present invention in an amount of 0.001 to 99.999 wt.-%, preferably from 0.01 to 99.99 wt.-%, more preferably from 0.1 to 99.9 wt.-%, in each case based on the total weight of the pharmaceutical composition.

The present invention also relates to a method of treating cancer, comprising administering to a patient suffering from cancer a therapeutically effective amount of the hydroxyalkyl starch conjugate as defined herein, or the hydroxyalkyl starch conjugate obtained or obtainable by the method according to the present invention, or the pharmaceutical composition according to the present invention.

The term “patient”, as used herein, relates to animals and, preferably, to mammals. More preferably, the patient is a rodent such as a mouse or a rat. Even more preferably, the patient is a primate. Most preferably, the patient is a human. It is, however, envisaged by the method of the present invention that the patient shall suffer from cancer.

The term “cancer”, as used herein, preferably refers to a proliferative disorder or disease caused or characterized by the proliferation of cells which have lost susceptibility to normal growth control. Preferably, the term encompasses tumors and any other proliferative disorders. Thus, the term is meant to include all pathological conditions involving malignant cells, irrespective of stage or of invasiveness. The term, preferably, includes solid tumors arising in solid tissues or organs as well as hematopoietic tumors (e.g. leukemias and lymphomas).

The cancer may be localized to a specific tissue or organ (e.g. in the breast, the prostate or the lung), and, thus, may not have spread beyond the tissue of origin. Furthermore the cancer may be invasive, and, thus may have spread beyond the layer of tissue in which it originated into the normal surrounding tissues (frequently also referred to as locally advanced cancer). Invasive cancers may or may not be metastatic. Thus, the cancer may be also metastatic. A cancer is metastatic, if it has spread from its original location to distant parts of the body. E.g., it is well known in the art that breast cancer cells may spread to another organ or body part, such as the lymph nodes.

Preferred cancers are acute lymphocytic leukemia (ALL), acute myeloid leukemia (AML), Hodgkin's disease, malignant lymphoma, soft tissue and bone sarcomas, thyroid cancer, small cell lung cancer, breast cancer, gastric cancer, ovarian cancer, bladder cancer, neuroblastoma, and Wilms' tumor.

Moreover, it is also envisaged that the cancer is selected from the group consisting of Acute Lymphoblastic Leukemia (adult), Acute Lymphoblastic Leukemia (childhood), Acute Myeloid Leukemia (adult), Acute Myeloid Leukemia (childhood), Adrenocortical Carcinoma, Adrenocortical Carcinoma (childhood), AIDS-Related Cancers, AIDS-Related Lymphoma, Anal Cancer, Appendix Cancer, Astrocytomas (childhood), Atypical Teratoid/Rhabdoid Tumor (childhood), Central Nervous System Cancer, Basal Cell Carcinoma, Bile Duct Cancer (Extrahepatic), Bladder Cancer, Bladder Cancer (childhood), Bone Cancer, Osteosarcoma and Malignant Fibrous Histiocytoma, Brain Stem Glioma (childhood), Brain Tumor (adult), Brain Tumor (childhood), Brain Stem Glioma (childhood), Central Nervous System Brain Tumor, Atypical Teratoid/Rhabdoid Tumor (childhood), Brain Tumor, Central Nervous System Embryonal Tumors (childhood), Astrocytomas (childhood) Brain Tumor, Craniopharyngioma Brain Tumor (childhood), Ependymoblastoma Brain Tumor (childhood), Ependymoma Brain Tumor (childhood), Medulloblastoma Brain Tumor (childhood), Medulloepitheliom Brain Tumor (childhood), Pineal Parenchymal Tumors of Intermediate Differentiation Brain Tumor (childhood), Supratentorial Primitive Neuroectodermal Tumors and Pineoblastoma Brain Tumor, (childhood), Brain and Spinal Cord Tumors (childhood), Breast Cancer, Breast Cancer (childhood), Breast Cancer (Male), Bronchial Tumors (childhood), Burkitt Lymphoma, Carcinoid Tumor (childhood), Carcinoid Tumor, Gastrointestinal, Carcinoma of Unknown Primary, Central Nervous System Atypical Teratoid/Rhabdoid Tumor (childhood), Central Nervous System Embryonal Tumors (childhood), Central Nervous System (CNS) Lymphoma, Primary Cervical Cancer, Cervical Cancer (childhood), Childhood Cancers, Chordoma (childhood), Chronic Lymphocytic Leukemia, Chronic Myelogenous Leukemia, Chronic Myeloproliferative Disorders, Colon Cancer, Colorectal Cancer (childhood), Craniopharyngioma (childhood), Cutaneous T-Cell Lymphoma, Embryonal Tumors, Central Nervous System (childhod), Endometrial Cancer, Ependymoblastoma (childhood), Ependymoma (childhood), Esophageal Cancer, Esophageal Cancer (childhood), Esthesioneuroblastoma (childhood), Ewing Sarcoma Family of Tumors, Extracranial Germ Cell Tumor (childhood), Extragonadal Germ Cell Tumor, Extrahepatic Bile Duct Cancer, Eye Cancer, Intraocular Melanoma, Eye Cancer, Retinoblastoma, Gallbladder Cancer, Gastric (Stomach) Cancer, Gastric (Stomach) Cancer (childhood), Gastrointestinal Carcinoid Tumor, Gastrointestinal Stromal Tumor (GIST), Gastrointestinal Stromal Cell Tumor (childhood), Germ Cell Tumor, Extracranial (childhood), Germ Cell Tumor, Extragonadal, Germ Cell Tumor, Ovarian, Gestational Trophoblastic Tumor, Glioma (adult), Glioma (childhood) Brain Stem, Hairy Cell Leukemia, Head and Neck Cancer, Heart Cancer (childhood), Hepatocellular (Liver) Cancer (adult) (Primary), Hepatocellular (Liver) Cancer (childhood) (Primary), Histiocytosis, Langerhans Cell, Hodgkin Lymphoma (adult), Hodgkin Lymphoma (childhood), Hypopharyngeal Cancer, Intraocular Melanoma, Islet Cell Tumors (Endocrine Pancreas), Kaposi Sarcoma, Kidney (Renal Cell) Cancer, Kidney Cancer (childhood), Langerhans Cell Histiocytosis, Laryngeal Cancer, Laryngeal Cancer (childhood), Leukemia, Acute Lymphoblastic (adult), Leukemia, Acute Lymphoblastic (childhood), Leukemia, Acute Myeloid (adult), Leukemia, Acute Myeloid (childhood), Leukemia, Chronic Lymphocytic, Leukemia, Chronic Myelogenous, Leukemia, Hairy Cell, Lip and Oral Cavity Cancer, Liver Cancer (adult) (Primary), Liver Cancer (childhood) (Primary), Non-Small Cell Lung Cancer, Small Cell Lung Cancer, Non-Hodgkin Lymphoma, (adult), Non-Hodgkin Lymphoma, (childhood), Primary Central Nervous System (CNS) Lymphoma, Waldenstrm, Macroglobulinemia, Malignant Fibrous Histiocytoma of Bone and Osteosarcoma, Medulloblastoma (childhood), Medulloepithelioma (childhood), Melanoma, Intraocular (Eye)Melanoma, Merkel Cell Carcinoma, Mesothelioma (adult) Malignant, Mesothelioma (childhood), Metastatic Squamous Neck Cancer with Occult Primary, Mouth Cancer, Multiple Endocrine Neoplasia Syndromes (childhood), Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia (adult) Acute, Myeloid Leukemia (childhood) Acute, Myeloma, Multiple, Nasal Cavity and Paranasal Sinus Cancer, Nasopharyngeal Cancer, Nasopharyngeal Cancer (childhood), Neuroblastoma, Oral Cancer (childhood), Lip and Oral Cavity Cancer, Oropharyngeal Cancer, Osteosarcoma and Malignant Fibrous, Histiocytoma of Bone, Ovarian Cancer (childhood), Ovarian Epithelial Cancer, Ovarian Germ Cell Tumor, Ovarian Low Malignant Potential Tumor, Pancreatic Cancer, Pancreatic Cancer (childhood), Pancreatic Cancer, Islet Cell Tumors, Papillomatosis (childhood), Paranasal Sinus and Nasal Cavity Cancer, Parathyroid Cancer, Penile Cancer, Pharyngeal Cancer, Pineal Parenchymal Tumors of Intermediate Differentiation (childhood), Pineoblastoma and Supratentorial Primitive Neuroectodermal Tumors (childhood), Pituitary Tumor, Plasma Cell Neoplasm/Multiple Myeloma, Pleuropulmonary Blastoma, Pregnancy and Breast Cancer, Primary Central Nervous System (CNS) Lymphoma, Prostate Cancer, Rectal Cancer, Renal Cell (Kidney) Cancer, Renal Pelvis and Ureter Transitional Cell Cancer, Respiratory Tract Cancer with Chromosome 15 Changes, Retinoblastoma, Rhabdomyosarcoma (childhood), Salivary Gland Cancer, Salivary Gland Cancer (childhood), Sarcoma, Ewing Sarcoma Family of Tumors, Kaposi Sarcoma, Soft Tissue (adult)Sarcoma, Soft Tissue (childhood)Sarcoma, Uterine Sarcoma, Szary Syndrome, Skin Cancer (Nonmelanoma), Skin Cancer (childhood), Skin Cancer (Melanoma), Merkel Cell Skin Carcinoma, Small Cell Lung Cancer, Small Intestine Cancer, Soft Tissue Sarcoma (adult), Soft Tissue Sarcoma (childhood), Squamous Cell Carcinoma, see Skin Cancer (Nonmelanoma), Stomach (Gastric) Cancer, Stomach (Gastric) Cancer (childhood), Supratentorial Primitive Neuroectodermal Tumors (childhood), Cutaneous T-Cell Lymphoma, Testicular Cancer, Testicular Cancer (childhood), Throat Cancer, Thymoma and Thymic Carcinoma, Thymoma and Thymic Carcinoma (childhood), Thyroid Cancer, Thyroid Cancer (childhood), Transitional Cell Cancer of the Renal Pelvis and Ureter, T Gestational rophoblastic Tumor, Unknown Primary Site, Carcinoma of adult, Unknown Primary Site, Cancer of (childhood), Unusual Cancers of childhood, Ureter and Renal Pelvis, Transitional Cell Cancer, Urethral Cancer, Uterine Cancer, Endometrial, Uterine Sarcoma, Vaginal Cancer, Vaginal Cancer (childhood), Vulvar Cancer and Waldenstrm Macroglobulinemia.

The terms “treating cancer” and “treatment of cancer”, preferably, refer to therapeutic measures, wherein the object is to prevent or to slow down (lessen) an undesired physiological change or disorder, such as the growth, development or spread of a hyperproliferative condition, such as cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. It is to be understood that a treatment can also mean prolonging survival as compared to expected survival if not receiving treatment.

The term “administering” as used herein, preferably, refers to the introduction of the hydroxyalkyl starch conjugate as defined herein, the hydroxyalkyl starch conjugate obtained or obtainable by the method according to the present invention, or the pharmaceutical composition according to the present invention into cancer patients. Methods for administering a particular compound are well known in the art and include parenteral, intravascular, paracanceral, transmucosal, transdermal, intramuscular (i.m.), intravenous (i.v.), intradermal, subcutaneous (s.c.), sublingual, intraperitoneal (i.p.), intraventricular, intracranial, intravaginal, intratumoral, and oral administration. It is to be understood that the route of administration may depend on the cancer to be treated. Preferably, the hydroxyalkyl starch conjugate as defined herein, the hydroxyalkyl starch conjugate obtained or obtainable by the method according to the present invention, or the pharmaceutical composition according to the present invention are administered parenterally. More preferably, it is administered intravenously. Preferably, the administration of a single dose of a therapeutically effective amount of the aforementioned compounds is over a period of 5 min to 5 h.

Preferably, the conjugates are administered together with a suitable carrier, and/or a suitable diluent, such as, preferably, a sterile solution for i.v., i.m., i.p. or s.c. application.

The term “therapeutically effective amount”, as used herein, preferably refers to an amount of the hydroxyalkyl starch conjugate as defined herein, the hydroxyalkyl starch conjugate obtained or obtainable by the method according to the present invention, or the pharmaceutical composition according to the present invention that (a) treats the cancer, (b) attenuates, ameliorates, or eliminates the cancer. More preferably, the term refers to the amount of the cytotoxic agent present in the hydroxyalkyl starch conjugate as defined herein, the hydroxyalkyl starch conjugate obtained or obtainable by the method according to the present invention, or the pharmaceutical composition according to the present invention that (a) treats the cancer, (b) attenuates, ameliorates, or eliminates the cancer. How to calculate the amount of a cytotoxic agent present in the aforementioned conjugates or pharmaceutical composition is described elsewhere herein. It is particularly envisaged that the therapeutically effective amount of the aforementioned compounds shall reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, at least to some extent, tumor growth; and/or relieve to some extent one or more of the symptoms associated with the cancer. Whether a particular amount of the aforementioned compounds exerts these effects (and, thus is pharmaceutically effective) can be determined by well known measures. Particularly, it can be determined by assessing cancer therapy efficacy. Cancer therapy efficacy, e.g., can be assessed by determining the time to disease progression and/or by determining the response rate. Thus, the required dosage will depend on the severity of the condition being treated, the patient's individual response, the method of administration used, and the like. The skilled person is able to establish a correct dosage based on his general knowledge.

Advantageously, it has been shown in the studies carried out in the context of the present invention that

i) the cytotoxic agent is less toxic when present in the conjugates described herein as compared to an agent not being present in a conjugate and/or
ii) that the use of said conjugate, or of the pharmaceutical composition comprising said conjugate allows for a more efficient treatment of cancer in a subject (see Example 2).

Moreover, the present invention relates to the hydroxyalkyl starch conjugate as defined above, or the hydroxyalkyl starch conjugate obtained or obtainable by the method according to the present invention, or the pharmaceutical composition according to the present invention for use as a medicament.

Moreover, the present invention relates to the hydroxyalkyl starch conjugate as defined above, or the hydroxyalkyl starch conjugate obtained or obtainable by the method according to the present invention, or the pharmaceutical composition according to the present invention for the treatment of cancer.

Also envisaged by the present invention is the hydroxyalkyl starch conjugate as defined above, or the hydroxyalkyl starch conjugate obtained or obtainable by the method according to the present invention, or the pharmaceutical composition according to the present invention for the treatment of cancer selected from the group consisting of acute lymphocytic leukemia (ALL), acute myeloid leukemia (AML), Hodgkin's disease, malignant lymphoma, soft tissue and bone sarcomas, thyroid cancer, small cell lung cancer, breast cancer, gastric cancer, ovarian cancer, bladder cancer, neuroblastoma, and Wilms' tumor.

Finally, the present invention pertains to the use of the hydroxyalkyl starch conjugate as defined above, or the hydroxyalkyl starch conjugate obtained or obtainable by the method according to the present invention, or the pharmaceutical composition according to the present invention for the manufacture of a medicament for the treatment of cancer. Preferably, the cancer is selected from the group consisting of acute lymphocytic leukemia (ALL), acute myeloid leukemia (AML), Hodgkin's disease, malignant lymphoma, soft tissue and bone sarcomas, thyroid cancer, small cell lung cancer, breast cancer, gastric cancer, ovarian cancer, bladder cancer, neuroblastoma, and Wilms' tumor.

How to administer the conjugates, compositions or medicaments has been explained elsewhere herein.

In the following especially preferred embodiments are described:
1. A hydroxyalkyl starch (HAS) conjugate comprising a hydroxyalkyl starch derivative and a cytotoxic agent, said conjugate having a structure according to the following formula

HAS′(-M)ₙ
wherein
M is a residue of a cytotoxic agent, the cytotoxic agent comprising a carbonyl group, HAS′ is a residue of the hydroxyalkyl starch derivative comprising at least one functional group X,
n is greater than or equal to 1, preferably in the range of from 3 to 200, more preferably in the range of from 3 to 100,
and wherein the cytotoxic agent is linked via the carbonyl function present in the cytotoxic agent to the functional group X comprised in the hydroxyalkyl starch derivative, wherein the linkage via the carbonyl function is a cleavable linkage, which is capable of being cleaved in vivo so as to release the cytotoxic agent, wherein HAS′ preferably has a mean molecular weight above the renal threshold.
2. The conjugate according to embodiment 1, wherein the hydroxyalkyl starch conjugate is a hydroxyethyl starch (HES) conjugate.
3. The conjugate according to embodiment 1 or 2, wherein the cytotoxic agent is an anthracycline, preferably wherein the cytotoxic agent is selected from the group consisting of daunorubicin, doxorubicin, epirubicin, idarubicin, valrubicin esorubicin, caminomycin, 4-demethoxy-4′-O-methyl doxorubicin, 4′-O-tetrahydropyranyl-doxorubicin, 3′-deamino-3′-(3″-cyano-4″-morpholinyl)doxorubicin and aclacinomycin and analogues thereof, wherein the cytotoxic agent is selected from the group consisting of daunomycine, daunorubicine, doxorubicine, epirubicine, idarubicine and valrubicine.
4. The conjugate according to any of embodiments 1 to 3, wherein the at least one functional group X comprised in HAS′ has the structure

-G′-NH—N═,
and wherein G′ is selected from the group consisting of —Y^{G}—C(=G)-, —SO₂—, aryl, and heteroaryl,
wherein G is O or S,
and wherein Y^{G} is —O—, —NH— or —NH—NH—.
5. The conjugate according to embodiment 4, wherein X has the structure —NH—NH—C(=G)-NH—N═, wherein G is O or S, preferably O.
6. The conjugate according to any of embodiments 1 to 5, wherein the hydroxyalkyl starch derivative comprises at least one structural unit according to the following formula (I)

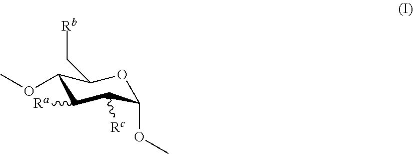

wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}—[F¹]ₚ-[L¹]_{q}—[F²]ᵣ-[L²]ᵥ-X—,
and wherein R^{w}, R^{x}, R^{y} and R^{z} are independently of each other selected from the group consisting of hydrogen and alkyl,
y is an integer in the range of from 0 to 20, preferably in the range of from 0 to 4,
x is an integer in the range of from 0 to 20, preferably in the range of from 0 to 4,
and wherein at least one of R^{a}, R^{b} and R^{c} is —[O—(CR^{w}R^{x})—(CR^{y}R^{z})]_{y}—[F¹]ₚ-[L¹]_{q}—[F²]ᵣ-[L²]ᵥ-X—,
and wherein X has the structure -G′—NH—N═, and wherein G′ is selected from the group consisting of —Y^{G}—C(=G)-, —SO₂—, aryl, and heteroaryl, wherein G is O or S, and
wherein Y^{G} is —O—, —NH— or —NH—NH—,
F¹ is selected from the group consisting of —O—, —S—, —NR^{Y7}— and —O—(C═Y⁶)—, wherein
Y⁶ is selected from the group consisting of NR^{Y6}, O and S, more preferably Y⁶ is O,
and wherein R^{Y6} is H or alkyl, preferably H, and wherein R^{Y7} is H or alkyl, preferably H, more preferably wherein F¹ is —O— or —O—(C═O)—,
p is 0 or 1,
and wherein L¹ is a linking moiety, preferably L¹ is selected from the group consisting of alkyl, alkenyl, alkylaryl, arylalkyl, aryl, heteroaryl, alkylheteroaryl and heteroarylalkyl,
q is 0 or 1, with the proviso that in case p is 0, q is 0,
F² is a functional group selected from the group consisting of —S—, —NH— and —NH—NH-T′-, wherein T′ is selected from the group consisting of —C(=T)-Y^{T}—, —SO₂—, aryl, and heteroaryl, and wherein T is O or S, and wherein Y^{T} is CH₂—, —O—, —NH— or —NH—NH—, preferably —O—, —NH— or —NH—NH—,
r is 0 or 1,
L² is a linking moiety, preferably L² is selected from the group consisting of alkyl, alkenyl, alkylaryl, arylalkyl, aryl, heteroaryl, alkylheteroaryl and heteroarylalkyl,
v is 0 or 1,
and wherein HAS″ is a remainder of HAS′.
7. The conjugate according to embodiment 6, wherein R^{a}, R^{b} and R^{c} are independently of each other selected from the group consisting of —O-HAS″, —[O—CH₂—CH₂]ₛ—OH and —[—O—CH₂—CH₂]ₜ—[F¹]ₚ-[L¹]_{q}—[F²]ᵣ-[L²]ᵥ-X—,
   and wherein s is in the range of from 0 to 4,
   and wherein t is in the range of from 0 to 4,
   wherein at least one of R^{a}, R^{b} and R^{c} is —[O—CH₂—CH₂]ₜ—[F¹]ₚ-[L¹]_{q}[F²]ᵣ-[L²]ᵥ-X—,
   and wherein HAS″ is a remainder of HAS′.
8. The conjugate according to embodiment 6 or 7, wherein p is 1 and wherein F¹ has the structure —O—C(═O)—.
9. The conjugate according to embodiment 8, wherein q is 0, and wherein
   (i) r and v are 0 and X has the structure

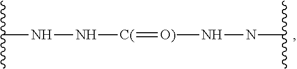

or
(ii) r and v are both 1, and wherein X has the structure -G′—NH—N═, and wherein G′ is selected from the group consisting of —Y^{G}—C(=G)-, —SO₂—, aryl, and heteroaryl, wherein G is O or S, and wherein Y^{G} is —O— or —NH—,
   and wherein F² has the structure —NH—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, NH—.
10. The conjugate according to embodiment 6 or 7, wherein p is 1 and q is 1 and wherein F¹ is —O— and wherein L¹ has a structure selected from the group consisting of —CH₂—CHOH—CH₂—, —CH₂—CH(CH₂OH)—, —CH₂—CHOH—CH₂—O—CH₂—CHOH—CH₂—, —CH₂—CH₂—CHOH—CH₂—, —CH₂—CH₂—CH₂—CHOH—CH₂—, more preferably from the group consisting of —CH₂—CHOH—CH₂—, —CH₂—CH(CH₂OH)—, —CH₂—CHOH—CH₂—O—CH₂—CHOH—CH₂—, more preferably from the group consisting of —CH₂—CHOH—CH₂— and —CH₂CH(CH₂OH)—,
   preferably wherein
   (i) r and v are 0, and X has the structure

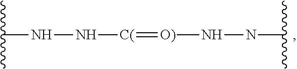

or
(ii) r and v are both 1, and wherein X has the structure -G′—NH—N═, and wherein G′ is selected from the group consisting of —Y^{G}—C(=G)-, —SO₂—, aryl, and heteroaryl, wherein G is O or S, and wherein Y^{G} is —O— or —NH— and
   wherein F² has the structure —NH—NH-T′-, wherein T′ is selected from the group consisting of —C(=T)-Y^{T}—, —SO₂—, aryl, and heteroaryl, and wherein T is O or S, and wherein Y^{T} is CH₂—, —O—, —NH—.
11. The conjugate according to embodiment to embodiment 6 or 7, wherein p and q are 0,
   (i) r and v are 0, and X has the structure

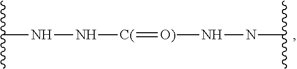

or
(ii) r and v are both 1, and wherein X has the structure -G′—NH—N═, and wherein G′ is selected from the group consisting of —Y^{G}—C(=G)-, —SO₂—, aryl, and heteroaryl, wherein G is O or S, and wherein Y^{G} is —O— or —NH—,
   and wherein F² has the structure —NH—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH—.
12. The conjugate according to any of embodiments 1 to 4, wherein the hydroxyalkyl starch derivative comprises at least one structural unit, preferably at least 3 structural units according to the following formula (I)

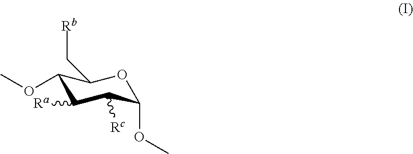

wherein R^{a}, R^{b} and R′ are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}—[F¹]ₚ-[L¹]_{q}—[F²]ᵣ-[L²]ᵥ-X—,
and wherein R^{w}, R^{x}, R^{y} and R^{z} are independently of each other selected from the group consisting of hydrogen and alkyl,
y is an integer in the range of from 0 to 20, preferably in the range of from 0 to 4,
x is an integer in the range of from 0 to 20, preferably in the range of from 0 to 4,
and wherein at least one of R^{a}, R^{b} and R^{c} is —[O—(CR^{w}R^{x})—(CR^{y}R^{z})]_{y}—[F¹]ₚ-[L¹]_{q}—[F²]ᵣ-[L²]ᵥ-X—,
and wherein X has the structure -G′—NH—N═, and wherein G′ is selected from the group consisting of —Y^{G}—C(=G)-, —SO₂—, aryl, and heteroaryl, wherein G is O or S, and wherein Y^{G} is —O—, —CH₂—, —NH— or —NH—NH—,
p is 1, and F¹ is —O—,
q is 1 and L¹ is a linking moiety selected from the group consisting of —CH₂—CHOH—CH₂—, —CH₂—CH(CH₂OH)—, —CH₂—CHOH—CH₂—O—CH₂—CHOH—CH₂—, —CH₂—CH₂—CHOH—CH₂—, —CH₂—CH₂—CH₂—CHOH—CH₂—,
r is 1, and F² is a functional group having a structure selected from the group consisting of —S—, —NH— and —NH—NH-T′-, wherein T′ is selected from the group consisting of —C(=T)-Y^{T}, —SO₂—, aryl, and heteroaryl, and wherein T is O or S, and
wherein Y^{T} is —CH₂—, —O—, —NH— or —NH—NH—, preferably —O—, —NH— or —NH—NH—,
and v is 1, and L² is a linking moiety, preferably L² is selected from the group consisting of alkyl, alkenyl, alkylaryl, arylalkyl, aryl, heteroaryl, alkylheteroaryl and heteroarylalkyl,
and wherein HAS″ is a remainder of HAS′.
13. The conjugate according to any of embodiments 1 to 4, wherein the hydroxyalkyl starch derivative comprises at least one structural unit, preferably at least 3 structural units according to the following formula (I)

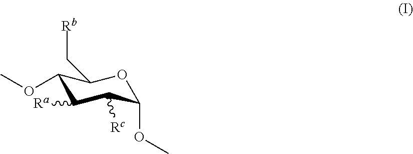

wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}—[F¹]ₚ-[L¹]_{q}—[F²]ᵣ[L²]ᵥ-X—,
and wherein R^{w}, R^{x}, R^{y} and R^{z} are independently of each other selected from the group consisting of hydrogen and alkyl,
y is an integer in the range of from 0 to 20, preferably in the range of from 0 to 4,
x is an integer in the range of from 0 to 20, preferably in the range of from 0 to 4,
and wherein at least one of R^{a}, R^{b} and R^{c} is —[O—(CR^{w}R^{x})—(CR^{y}R^{z})]_{y}—[F¹]ₚ-[L¹]_{q}—[F²]ᵣ-[L²]ᵥ-X—,
and wherein X has the structure -G′—NH—N═, and wherein G′ is selected from the group consisting of —Y⁶—C(=G)-, —SO₂—, aryl, and heteroaryl, wherein G is O or S, and
wherein Y⁶ is —O—, —CH₂—, —NH— or —NH—NH—,
p is 0,
q is 0,
r is 1, and F² is a functional group having the structure —NH—NH-T′-, wherein T′ is selected from the group consisting of —C(=T)-Y^{T}, —SO₂—, aryl, and heteroaryl, and wherein T is O or S, and wherein Y^{T} is —CH₂—, —O—, —NH— or —NH—NH—, preferably —O—, —NH— or —NH—NH—,
and v is 1, and L² is a linking moiety, preferably L² is selected from the group consisting of alkyl, alkenyl, alkylaryl, arylalkyl, aryl, heteroaryl, alkylheteroaryl and heteroarylalkyl,
and wherein HAS″ is a remainder of HAS′.
14. The conjugate according to any of embodiments 1 to 17, wherein the hydroxyalkyl starch derivative has a mean molecular weight MW greater than or equal to 60 kDa and a molar substitution MS in the range of from 0.70 to 1.45.
15. The conjugate according to any of embodiments 1 to 14, wherein the conjugate has a structure according to the following formula:

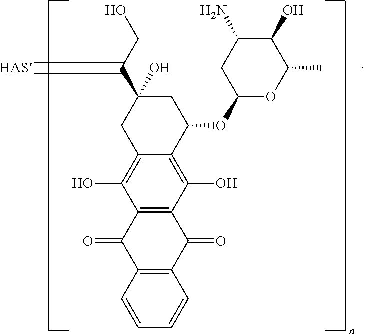

16. A method for preparing a hydroxyalkyl starch (HAS) conjugate comprising a hydroxyalkyl starch derivative and a cytotoxic agent, said conjugate having a structure according to the following formula

HAS′(-M)ₙ
wherein M is a residue of a cytotoxic agent, wherein the cytotoxic agent comprises a carbonyl group,
HAS′ is a residue of the hydroxyalkyl starch derivative comprising at least one functional group X,
n is greater than or equal to 1, preferably in the range of from 3 to 200, more preferably in the range of from 3 to 100,
and wherein the cytotoxic agent M is linked via the carbonyl function present in the cytotoxic agent M to a functional group X comprised in the hydroxyalkyl starch derivative, wherein the linkage via the carbonyl function is a cleavable linkage, which is capable of being cleaved in vivo so as to release the cytotoxic agent M, said method comprising
(a) providing a hydroxyalkyl starch (HAS) derivative, said HAS derivative comprising a functional group Z¹; and providing a cytotoxic agent comprising a carbonyl group;
(b) coupling the HAS derivative to the cytotoxic agent, wherein the functional group Z¹ comprised in the hydroxyalkyl starch derivative is coupled directly to the carbonyl group of the cytotoxic agent thereby forming the functional group —X—.
17. The method according to embodiment 16, wherein the functional group Z¹ has the structure
   -G′—NH—NH₂, and wherein G′ is selected from the group consisting of —Y^{G}—C(=G)-, —SO₂—, aryl, and heteroaryl, wherein G is O or S, and wherein Y^{G} is —O—, —NH— or —NH—NH—.
18. The method according to any of embodiments 16 or 17, wherein the hydroxyalkyl starch derivative provided in step (a) comprises at least one structural unit, preferably 3 to 200 structural units, more preferably in the range of from 3 to 100,
   according to the following formula (I)

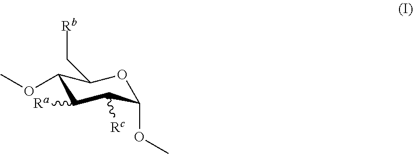

wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}—[F¹]ₚ-[L¹]_{q}—[F²]ᵣ-[L²]ᵥ-Z¹,
and wherein R^{w}, R^{x}, R^{y} and R^{z} are independently of each other selected from the group consisting of hydrogen and alkyl,
y is an integer in the range of from 0 to 20, preferably in the range of from 0 to 4,
x is an integer in the range of from 0 to 20, preferably in the range of from 0 to 4,
and wherein at least one of R^{a}, R^{b} and R^{c} is —[O—(CR^{w}R^{x})—(CR^{y}R^{z})]_{y}—[F¹]ₚ-[L¹]_{q}—[F²]ᵣ-[L²]ᵥ-Z¹,
wherein F¹ is selected from the group consisting of —O—, —S—, —NR^{Y7}— and —O—(C═Y⁶)—,
wherein Y⁶ is selected from the group consisting of NR^{Y6}, O and S, more preferably Y⁶ is O, and wherein R^{Y6} is H or alkyl, preferably H, and wherein R^{Y7} is H or alkyl, preferably H, more preferably wherein F¹ is —O— or —O—(C═O)—,
p is 0 or 1,
and wherein L¹ is a linking moiety, preferably L¹ is selected from the group consisting of alkyl, alkenyl, alkylaryl, arylalkyl, aryl, heteroaryl, alkylheteroaryl and heteroarylalkyl,
q is 0 or 1, with the proviso that in case p is 0, q is 0,
F² is a functional group selected from the group consisting of —S—, —NH— and —NH—NH-T′-, wherein T′ is selected from the group consisting of —C(=T)-Y^{T}-, —SO₂—, aryl, and heteroaryl, and wherein T is O or S, and wherein Y^{T} is —CH₂—, —O—, —NH— or —NH—NH—, preferably —O—, —NH— or —NH—NH—,
r is 0 or 1,
L² is a linking moiety, preferably L² is selected from the group consisting of alkyl, alkenyl, alkylaryl, arylalkyl, aryl, heteroaryl, alkylheteroaryl and heteroarylalkyl,
v is 0 or 1,
and wherein HAS″ is a remainder of HAS′,
and wherein step (a) comprises
(a1) providing a hydroxyalkyl starch, preferably having a mean molecular weight MW above the renal threshold, preferably greater than or equal to 60 kDa and a molar substitution MS in the range of from 0.6 to 1.5, comprising the structural unit according to the following formula (II)

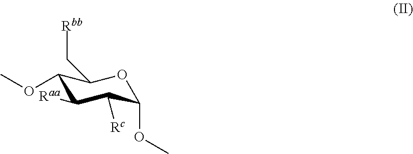

wherein R^{aa}, R^{bb} and R^{cc} are, independently of each other, selected from the group consisting of —O-HAS″ and —[O—(CR^{w}R^{x})—(CR^{y}R^{z})]ₓ—OH,
and wherein R^{w}, R^{x}, R^{y} and R^{z} are independently of each other selected from the group consisting of hydrogen and alkyl, and wherein
x is an integer in the range of from 0 to 20, preferably in the range of from 0 to 4,
(a2) introducing at least one functional group Z¹ into HAS by
   (i) coupling the hydroxyalkyl starch via at least one hydroxyl group comprised in HAS to at least one suitable linker comprising the functional group Z¹ or a precursor of the functional group Z¹, or
   (ii) displacing at least one hydroxyl group comprised in HAS in a substitution reaction with a suitable linker comprising the functional group Z¹ or a precursor thereof.
19. The method according to embodiment 18, wherein the HAS derivative provided in step (a2) comprises at least one structural unit according to the following formula (I)

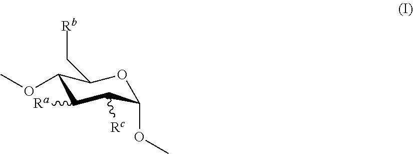

wherein R^{a}, R^{b} and R^{c} are independently of each other selected from the group consisting of —O-HAS″, —[O—CH₂—CH₂]ₛ—OH and —[O—CH₂—CH₂]ₜ—[F¹]ₚ-[L¹]_{q}—[F²]ᵣ-[L²]ᵥ-Z¹,
and wherein at least one of R^{a}, R^{b} and R^{c} is —[O—CH₂—CH₂]ₜ—[F¹]ₚ-[L¹]_{q}—[F²]ᵣ-[L²]ᵥ-Z¹,
s is in the range of from 0 to 4,
and t is in the range of from 0 to 4.
20. The method according to embodiment 18 or 19, wherein step (a2)(i) comprises
   (aa) activating at least one hydroxyl group comprised in the hydroxyalkyl starch with a reactive carbonyl compound having the structure R**—(C═O)R*, wherein R* and R** may be the same or different, and wherein R* and R** are both leaving groups, wherein upon activation a hydroxyalkyl starch derivative comprising at least one structural unit according to the following formula (Ib)

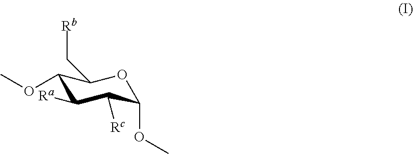

is formed, in which R^{a}, R^{b} and R^{c} are independently of each other selected from the group consisting of —O-HAS″, —[O—CH₂—CH₂]ₛ—OH, and —[O—CH₂—CH₂]ₜ—O—C(═O)R^{d},
wherein s is in the range of from 0 to 4,
and wherein t is in the range of from 0 to 4,
wherein at least one of R^{a}, R^{b} and R^{c} comprises the group —[O—CH₂—CH₂]ₜ—O—C(═O)R^{d}, and
(bb) reacting the activated hydroxyalkyl starch according to step (aa) with the suitable linker comprising the functional group Z¹ or a precursor of the functional group Z¹.
21. The method according to embodiment 25, wherein the reactive carbonyl compound having the structure R**—(C═O)R* is selected from the group consisting of phosgene and the like (diphosgene, triphosgene), chloroformates such as p-nitrophenylchloroformate, pentafluorophenylchloroformate, phenylchloroformate, methyl- and ethylchloroformate, carbonic acid esters such as N,N′-disuccinimidyl carbonate, sulfo-N,N′-disuccinimidyl carbonate, dibenzotriazol-1-yl carbonate and carbonyldiimidazol.
22. The method according to embodiment 20 or 21, wherein in step (bb), the activated hydroxyalkyl starch derivative is reacted with a linker having the structure Z²-[L²]ᵥ-Z¹, wherein
   v is 1, and Z² has a structure according to the formula H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or —NH—NH—, preferably —O—, —NH— or —NH—NH—,
      and wherein upon reaction of Z²-[L²]ᵥ-Z¹ with the group —O—C(═O)—R* comprised in the hydroxyalkyl starch derivative, the structural unit —[F¹]ₚ-[L¹]_{q}—[F²]ᵣ-[L²]ᵥ-Z¹ is formed, with q being 1, and with F¹ being —O—C(═O)— and with F² being —NH—NH-T′-, or wherein
   v is 0, Z² is H, and Z¹ has the structure

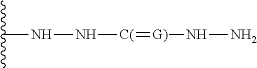

and wherein upon reaction of Z²-[L²]ᵥ-Z¹ with the group —O—C(═O)—R* comprised in the hydroxyalkyl starch derivative, the structural unit —[F¹]ₚ-[L¹]_{q}—[F²]ᵣ-[L²]ᵥ-Z¹ is formed, with q, r and v being 0.
23. The method according to embodiment 18 or 19, wherein (a2)(i) comprises
   (I) coupling the hydroxyalkyl starch via at least one hydroxyl group comprised in the hydroxyalkyl starch with a first linker comprising a functional group K², K² being capable of being reacted with a hydroxyl group of the hydroxylalkyl starch, thereby forming a covalent linkage, the first linker further comprising a functional group W, with W being a precursor of the functional group Z¹, and wherein the functional group W is an epoxide or a group which is transformed in a further step to give an epoxide.
24. The method according to embodiment 23, wherein the first linker has a structure according to the formula K²-L^{W}W, wherein
   K² is a functional group capable of being reacted with a hydroxyl group of the hydroxyalkyl starch,
   L^{W} is a linking moiety,
   wherein upon reaction of the hydroxyalkyl starch with the first linker, a hydroxyalkyl starch derivative is formed comprising at least one structural unit according to the following formula (I)

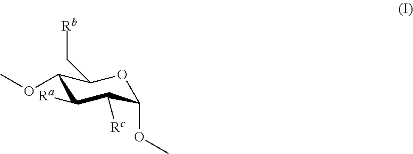

wherein R^{a}, R^{b} and R^{c} are independently of each other selected from the group consisting of —O-HAS″, —[O—CH₂—CH₂]ₛ—OH and —[O—CH₂—CH₂]ₜ—[F¹]ₚ-L^{W}-W,
and wherein at least one of R^{a}, R^{b} and R^{c} is —[O—CH₂—CH₂]—[F¹]ₚ-L^{W}-W,
s is in the range of from 0 to 4,
and t is in the range of from 0 to 4,
p is 1,
and wherein F¹ is a functional group which is formed upon reaction of K² with a hydroxyl group of the hydroxyalkyl starch, wherein F¹ is preferably —O—,
and wherein HAS″ is a remainder of HAS′.
25. The method according to embodiment 23 or 24, wherein W is an alkenyl group and the method further comprises
   (II) oxidizing the alkenyl group W to give the epoxide, wherein as oxidizing agent, potassium peroxymonosulfate (Oxone®) is preferably employed,
   and wherein in step (II), preferably a hydroxyalkyl starch derivative comprising at least one structural unit according to the following formula (I)

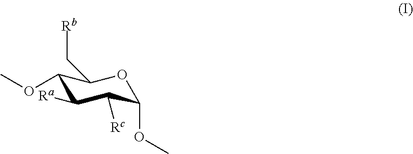

is formed, wherein R^{a}, R^{b} and R^{c} are independently of each other selected from the group consisting of —O-HAS″, —[O—CH₂—CH₂]ₛ—OH and

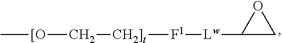

and wherein at least one of R^{a}, R^{b} and R^{c} is

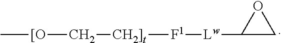

26. The method according to embodiment 25, the method comprising
   (III) reacting the epoxide with a linker having the structure Z²-[L²]ᵥ-Z¹ or Z²-[L²]ᵥ-Z¹* —PG, wherein PG is a suitable protecting group, and wherein
      and Z¹* being the protected form of the functional group Z¹.
      v is 1, and Z² has a structure selected from the group consisting of HS—, H₂N— and H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or —NH—NH—, preferably —O—, —NH— or —NH—NH—,
      and wherein upon reaction the linker with the structural unit

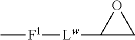

comprised in the hydroxyalkyl starch derivative, the structural unit —[F¹]ₚ-[L¹]_{q}—[F²]ᵣ-[L²]ᵥ-Z¹ is formed, with q being 1, with p being 1 and with F¹ being —O—, and wherein r is 1, and F² is selected from the group consisting of —S—, —HN— and —NH—NH-T′, or wherein
v is 0, and Z² is H and Z¹ has the structure

[symbol]

NH—NH—C(=G)-NH—NH₂
and wherein upon reaction of Z²-[L²]ᵥ-Z¹ with the structural unit

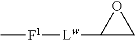

comprised in the hydroxyalkyl starch derivative, the structural unit —[F¹]ₚ-[L¹]_{q}—[F²]ᵣ-[L²]ᵥ-Z¹ is formed, with q being 1, and with r being 0.
27. The method according to embodiment 18, wherein in step (a2)(ii), prior to the displacement of the hydroxyl group, a group R^{L} is added to at least one hydroxyl group, thereby generating a group —O—R^{L}, wherein —O—R^{L} is a leaving group, in particular an —O-Mesyl (-OMs) or an —O-Tosyl (—O-Ts) group.
28. The method according to embodiment 18 or 27, wherein the suitable linker according to step (a2)(ii) has the structure Z²-[L²]ᵥ-Z¹ wherein
   v is 1, and Z² has a structure according to the formula H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or —NH—NH—, preferably —O—, —NH— or —NH—NH—,
      and wherein upon reaction the structural unit —[F¹]ₚ-[L¹]_{q}—[F²]ᵣ-[L²]ᵥ-Z¹ is formed, with p and q being 0, with r being 1 and with F² being selected from the group consisting of —S—, —NH— and —NH—NH-T′-, or wherein
   v is 0 and Z² is H and Z¹ has the structure

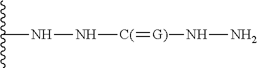

and wherein upon reaction the structural unit —[F¹]ₚ-[L¹]_{q}—[F²]ᵣ-[L²]ᵥ-Z¹ is formed, with p, q, r and v being 0.
29. The method according to any of embodiments 16 to 28, wherein the cytotoxic agent is an anthracycline, preferably, wherein the cytotoxic agent is selected from the group consisting of daunorubicin, doxorubicin, epirubicin, idarubicin and valrubicin.
30. A hydroxyalkyl starch conjugate obtained or obtainable by a method according to any of embodiments 18 to 28.
31. A pharmaceutical composition comprising a conjugate according to any of embodiments 1 to 15 or according to embodiment 30.

32. A hydroxyalkyl starch conjugate according to any of embodiments 1 to 15 or according to embodiment 30, or a pharmaceutical composition according to embodiment 31 for use as medicament.
33. A hydroxyalkyl starch conjugate according to any of embodiments 1 to 15 or according to embodiment 30, or a pharmaceutical composition according to embodiment 31 for the treatment of cancer.
34. A hydroxyalkyl starch conjugate according to any of embodiments 1 to 15 or according to embodiment 30, or a pharmaceutical composition according to embodiment 31 for the treatment of cancer selected from the group consisting of acute lymphocytic leukemia (ALL), acute myeloid leukemia (AML), Hodgkin's disease, malignant lymphoma, soft tissue and bone sarcomas, thyroid cancer, small cell lung cancer, breast cancer, gastric cancer, ovarian cancer, bladder cancer, neuroblastoma, and Wilms' tumor.
35. Use of a hydroxyalkyl starch conjugate according to any of embodiments 1 to 15 or according to embodiment 30, or a pharmaceutical composition according to embodiment 31 for the manufacture of a medicament for the treatment of cancer.
36. Use of a hydroxyalkyl starch conjugate according to embodiment 35, wherein the cancer is selected from the group consisting acute lymphocytic leukemia (ALL), acute myeloid leukemia (AML), Hodgkin's disease, malignant lymphoma, soft tissue and bone sarcomas, thyroid cancer, small cell lung cancer, breast cancer, gastric cancer, ovarian cancer, bladder cancer, neuroblastoma, and Wilms' tumor.
37. A method of treating a patient suffering from cancer comprising administering a therapeutically effective amount of a hydroxyalkyl starch conjugate according to any of embodiments 1 to 15 or according to embodiment 30, or a pharmaceutical composition according to embodiment 31.
38. The method of embodiment 37 wherein the patient suffers from a cancer being selected from the group consisting of acute lymphocytic leukemia (ALL), acute myeloid leukemia (AML), Hodgkin's disease, malignant lymphoma, soft tissue and bone sarcomas, thyroid cancer, small cell lung cancer, breast cancer, gastric cancer, ovarian cancer, bladder cancer

### DESCRIPTION OF THE FIGURES

FIG. 1: Time course of the median RTV values after administering Doxorubicin conjugate CDx1 (dosage 8 and 20 mg/kg body weight; tumor model MT-3)

FIG. 1 shows the time course of the relative tumor volume of human breast cancer carcinoma MT-3 xenografts growing in nude mice treated with conjugate CDx1 vs. mice in the control group (untreated mice (saline)) as well as vs. mice treated with Doxorubicin.

The following symbols are used:

▪=saline, ★=Doxorubin, ∇=CDx1, 8 mg/kg, Δ=CDx1, 20 mg/kg.

The X-axis shows the time after start [d], the Y-axis shows the relative tumor volume, RTV [%].

Each measurement was carried out with a group of 8 mice. The conjugate CDx1 was administered twice at a dosage of 8 mg/kg body weight on day 7 and on day 14 and—at a parallel study—twice at a dosage of 20 mg/kg body weight on day 7 and day 21. Doxorubicin was administered 2 times at a dosage of 8 mg/kg body weight on days 7 and 14. Median values are given. Further details are given in Table 10.

FIG. 2: Time course of the body weight change after administering Doxorubicin conjugate CDx1 (dosage 8 and 20 mg/kg body weight; tumor model MT-3)

FIG. 2 shows the time course of the body weight change in nude mice bearing human breast cancer carcinoma MT-3 xenografts treated with conjugate CDx1 vs. mice in the control group (untreated mice (saline)) as well as vs. mice treated with Doxorubicin.

The following symbols are used:

▪=saline, ∇=CDx1, 8 mg/kg, Δ=CDx1, 20 mg/kg, ★=Doxorubicin.

The X-axis shows the time after start [d], the Y-axis shows the body weight change, BWC [%].

Each measurement was carried out with a group of 8 mice. The conjugate CDx1 was administered twice at a dosage of 8 mg/kg body weight on day 7 and on day 14 and—at a parallel study—twice at a dosage of 20 mg/kg body weight on day 7 and day 21. Doxorubicin was administered 2 times at a dosage of 8 mg/kg body weight on days 7 and 14. Median values are given. Further details are given in Table 10.

FIG. 3: Time course of the median RTV values after administering Doxorubicin conjugates CDx1 and CDx15 (dosage 8 and 20 mg/kg body weight; tumor model MT-3)

FIG. 3 shows the time course of the relative tumor volume of human breast cancer carcinoma MT-3 xenografts growing in nude mice treated with conjugates CDx1 and CDx15 vs. mice in the control group (untreated mice (saline)) as well as vs. mice treated with Doxorubicin.

The following symbols are used:

▪=saline, ★=Doxorubicin, ∇=CDx1, Δ=CDx1*, ⋄CDx15.

The X-axis shows the time after start [d], the Y-axis shows the relative tumor volume, RTV [%].

Each measurement was carried out with a group of 8 mice. The conjugate CDx1 was administered once at a dosage of 20 mg/kg body weight on day 10 and—at a parallel study—twice at a dosage of 20 mg/kg body weight on day 10 and day 17, depicted as CDx1*. CDx15 was administered once at a dosage of 8 mg/kg body weight on day 10. Doxorubicin was administered once at a dosage of 8 mg/kg body weight on day 10. Median values are given. Further details are given in Table 11.

FIG. 4: Time course of the body weight change after administering Doxorubicin conjugates CDx1 and CDx15 (dosage 8 and 20 mg/kg body weight; tumor model MT-3)

FIG. 4 shows the time course of the body weight change in nude mice bearing human breast cancer carcinoma MT-3 xenografts treated with conjugates CDx1 and CDx15 vs. mice in the control group (untreated mice (saline)) as well as vs. mice treated with Doxorubicin.

The following symbols are used:

▪=saline, ★=Doxorubicin, ∇=CDx1, Δ=CDx1*, ⋄CDx15.

The X-axis shows the time after start [d], the Y-axis shows the body weight change, BWC [%].

Each measurement was carried out with a group of 8 mice. The conjugate CDx1 was administered once at a dosage of 20 mg/kg body weight on day 10 and—at a parallel study—twice at a dosage of 20 mg/kg body weight on day 10 and day 17, depicted as CDx1*. CDx15 was administered once at a dosage of 8 mg/kg body weight on day 10. Doxorubicin was administered once at a dosage of 8 mg/kg body weight on day 10. Median values are given. Further details are given in Table 11.

FIG. 5: Time course of the median RTV values after administering Doxorubicin conjugates CDx6, CDx10, CDx11 and CDx14 (dosage 20 mg/kg body weight; tumor model MT-3)

FIG. 5 shows the time course of the relative tumor volume of human breast cancer carcinoma MT-3 xenografts growing in nude mice treated with conjugates CDx6, CDx10, CDx11 and CDx14 vs. mice in the control group (untreated mice (saline)) as well as vs. mice treated with Doxorubicin.

The following symbols are used:

▪=saline, ★=Doxorubicin, ◯=CDx10, Δ=CDx6, ⋄=CDx14, ∇=CDx11.

The X-axis shows the time after start [d], the Y-axis shows the relative tumor volume, RTV [%].

Each measurement was carried out with a group of 8 mice. The conjugates CDx6, CDx10, CDx11 and CDx14 were administered once at a dosage of 20 mg/kg body weight on day 7. Doxorubicin was administered once at a dosage of 8 mg/kg body weight on day 7. Median values are given. Further details are given in Table 12.

FIG. 6: Time course of the median RTV values after administering Doxorubicin conjugates CDx8, CDx9, CDx12 and CDx13 (dosage 20 mg/kg body weight; tumor model MT-3)

FIG. 6 shows the time course of the relative tumor volume of human breast cancer carcinoma MT-3 xenografts growing in nude mice treated with conjugates CDx8, CDx9, CDx12 and CDx13 vs. mice in the control group (untreated mice (saline)) as well as vs. mice treated with Doxorubicin.

The following symbols are used:

▪=saline, ★=Doxorubicin, ◯=CDx8, Δ=CDx9, ⋄CDx12, ∇=CDx13.

The X-axis shows the time after start [d], the Y-axis shows the relative tumor volume, RTV [%].

Each measurement was carried out with a group of 8 mice. The conjugates CDx8, CDx9, CDx12 and CDx13 were administered once at a dosage of 20 mg/kg body weight on day 7. Doxorubicin was administered once at a dosage of 8 mg/kg body weight on day 7. Median values are given. Further details are given in Table 12.

FIG. 7: Time course of the body weight change after administering Doxorubicin conjugates CDx6, CDx10, CDx11 and CDx14 (dosage 20 mg/kg body weight; tumor model MT-3)

FIG. 7 shows the time course of the body weight change in nude mice bearing human breast cancer carcinoma MT-3 xenografts treated with conjugates CDx6, CDx10, CDx11 and CDx14 vs. mice in the control group (untreated mice (saline)) as well as vs. mice treated with Doxorubicin.

The following symbols are used:

▪=saline, ★=Doxorubicin, ◯=CDx10, Δ=CDx6, ⋄CDx14, ∇=CDx11.

The X-axis shows the time [d], the Y-axis shows the body weight change, BWC [%].

Each measurement was carried out with a group of 8 mice. The conjugates CDx6, CDx10, CDx11 and CDx14 were administered once at a dosage of 20 mg/kg body weight on day 7. Doxorubicin was administered once at a dosage of 8 mg/kg body weight on day 7. Median values are given. Further details are given in Table 12.

FIG. 8: Time course of the body weight change after administering Doxorubicin conjugates CDx8, CDx9, CDx12 and CDx13 (dosage 20 mg/kg body weight; tumor model MT-3)

FIG. 8 shows the time course of the body weight change in nude mice bearing human breast cancer carcinoma MT-3 xenografts treated with conjugates CDx8, CDx9, CDx12 and CDx13 vs. mice in the control group (untreated mice (saline)) as well as vs. mice treated with Doxorubicin.

The following symbols are used:

▪=saline, ★=Doxorubicin, ◯=CDx8, Δ=CDx9, ⋄CDx12, ∇=CDx13.

The X-axis shows the time [d], the Y-axis shows the body weight change, BWC [%].

Each measurement was carried out with a group of 8 mice. The conjugates CDx8, CDx9, CDx12 and CDx13 were administered once at a dosage of 20 mg/kg body weight on day 7. Doxorubicin was administered once at a dosage of 8 mg/kg body weight on day 7. Median values are given. Further details are given in Table 12.

FIG. 9: Time course of the median RTV values after administering Doxorubicin conjugates CDx6 and CDx8 (dosage 20 mg/kg body weight; tumor model ovcar-3)

FIG. 9 shows the time course of the relative tumor volume of human ovarian cancer carcinoma ovcar-3 xenografts growing in nude mice treated with conjugates CDx6 and CDx8 vs. mice in the control group (untreated mice (saline)) as well as vs. mice treated with Doxorubicin.

The following symbols are used:

▪=saline, ★=Doxorubicin, Δ=CDx6, ∇=CDx8.

The X-axis shows the time after start [d], the Y-axis shows the relative tumor volume, RTV [%].

Each measurement was carried out with a group of 7 mice. The conjugates CDx6 and CDx8 were administered once at a dosage of 20 mg/kg body weight on day 5. Doxorubicin was administered once at a dosage of 8 mg/kg body weight on day 5. Median values are given. Further details are given in Table 13.

FIG. 10: Time course of the body weight change after administering Doxorubicin conjugates CDx6 and CDx8 (dosage 20 mg/kg body weight; tumor model ovcar-3)

FIG. 10 shows the time course of the body weight change in nude mice bearing human ovarian cancer carcinoma ovcar-3 xenografts treated with conjugates CDx6 and CDx8 vs. mice in the control group (untreated mice (saline)) treated with conjugates CDx6 and CDx8 vs. mice in the control group (untreated mice (saline)) as well as vs. mice treated with Doxorubicin.

The following symbols are used:

▪=saline, ★=Doxorubicin, Δ=CDx6, ∇=CDx8.

The X-axis shows the time after start [d], the Y-axis shows the body weight change, BWC [%].

Each measurement was carried out with a group of 7 mice. The conjugates CDx6 and CDx8 were administered once at a dosage of 20 mg/kg body weight on day 5. Doxorubicin was administered once at a dosage of 8 mg/kg body weight on day 5. Median values are given. Further details are given in Table 13.

FIG. 11: Time course of the median RTV values after administering Doxorubicin conjugates CDx6 and CDx8 (dosage 20 mg/kg body weight; tumor model MT3-ADR)

FIG. 9 shows the time course of the relative tumor volume of human mamma carcinoma MT3-ADR xenografts growing in nude mice treated with conjugates CDx6 and CDx8 vs. mice in the control group (untreated mice (saline)) as well as vs. mice treated with Doxorubicin.

The following symbols are used:

▪=saline, ★=Doxorubicin, Δ=CDx6, ∇=CDx8.

The X-axis shows the time after start [d], the Y-axis shows the relative tumor volume, RTV [%].

Each measurement was carried out with a group of 7 mice. The conjugates CDx6 and CDx8 were administered once at a dosage of 20 mg/kg body weight on day 10.

Doxorubicin was administered once at a dosage of 8 mg/kg body weight on day 10. Median values are given. Further details are given in Table 14.

FIG. 12: Time course of the body weight change after administering Doxorubicin conjugates CDx6 and CDx8 (dosage 20 mg/kg body weight; tumor model MT3-ADR)

FIG. 12 shows the time course of the body weight change in nude mice bearing human mamma carcinoma MT3-ADR xenografts treated with conjugates CDx6 and CDx8 vs. mice in the control group (untreated mice (saline)) treated with conjugates CDx6 and CDx8 vs. mice in the control group (untreated mice (saline)) as well as vs. mice treated with Doxorubicin.

The following symbols are used:

▪=saline, ★=Doxorubicin, Δ=CDx6, ∇=CDx8.

The X-axis shows the time after start [d], the Y-axis shows the body weight change, BWC [%].

Each measurement was carried out with a group of 7 mice. The conjugates CDx6 and CDx8 were administered once at a dosage of 20 mg/kg body weight on day 10. Doxorubicin was administered once at a dosage of 8 mg/kg body weight on day 10. Median values are given. Further details are given in Table 14.

FIG. 13: Time course of the median RTV values after administering Doxorubicin conjugates CDx5, CDx6, CDx7 and CDx8 (dosage 20 mg/kg body weight; tumor model MT-3)

FIG. 13 shows the time course of the relative tumor volume of human breast cancer carcinoma MT-3 xenografts growing in nude mice treated with conjugates CDx5, CDx6, CDx7 and CDx8 vs. mice in the control group (untreated mice (saline)) as well as vs. mice treated with Doxorubicin.

The following symbols are used:

▪=saline, ★=Doxorubicin, Δ=CDx5, ◯=CDx6, ⋄CDx7, ∇=CDx8.

The X-axis shows the time after start [d], the Y-axis shows the relative tumor volume, RTV [%].

Each measurement was carried out with a group of 8 mice. The conjugates CDx5, CDx6, CDx7 and CDx8 were administered once at a dosage of 20 mg/kg body weight on day 7. Doxorubicin was administered once at a dosage of 8 mg/kg body weight on day 7. Median values are given. Further details are given in Table 15.

FIG. 14: Time course of the body weight change after administering Doxorubicin conjugates CDx5, CDx6, CDx7 and CDx8 (dosage 20 mg/kg body weight; tumor model MT-3)

FIG. 14 shows the time course of the body weight change in nude mice bearing human breast cancer carcinoma MT-3 xenografts treated with conjugates CDx5, CDx6, CDx7 and CDx8 vs. mice in the control group (untreated mice (saline)) as well as vs. mice treated with Doxorubicin.

The following symbols are used:

▪=saline, ★=Doxorubicin, Δ=CDx5, ◯=CDx6, ⋄CDx7, ∇=CDx8.

The X-axis shows the time after start [d], the Y-axis shows the body weight change, BWC [%].

Each measurement was carried out with a group of 8 mice. The conjugates CDx5, CDx6, CDx7 and CDx8 were administered once at a dosage of 20 mg/kg body weight on day 7. Doxorubicin was administered once at a dosage of 8 mg/kg body weight on day 7. Median values are given. Further details are given in Table 15.

FIG. 15: Time course of the median RTV values after administering Doxorubicin conjugates CDx1, CDx2, CDx3 and CDx4 (dosage 20 mg/kg body weight; tumor model MT-3)

FIG. 15 shows the time course of the relative tumor volume of human breast cancer carcinoma MT-3 xenografts growing in nude mice treated with conjugates CDx1, CDx2, CDx3 and CDx4 vs. mice in the control group (untreated mice (saline)) as well as vs. mice treated with Doxorubicin.

The following symbols are used:

▪=saline, ★=Doxorubicin, ⋄CDx1, ∇=CDx2, ◯=CDx3, Δ=CDx4.

The X-axis shows the time after start [d], the Y-axis shows the relative tumor volume, RTV [%].

Each measurement was carried out with a group of 8 mice. The conjugates CDx1, CDx2, CDx3 and CDx4 were administered once at a dosage of 20 mg/kg body weight on day 7. Doxorubicin was administered once at a dosage of 8 mg/kg body weight on day 7. Median values are given. Further details are given in Table 16.

FIG. 16: Time course of the body weight change after administering Doxorubicin conjugates CDx1, CDx2, CDx3 and CDx4 (dosage 20 mg/kg body weight; tumor model MT-3)

FIG. 16 shows the time course of the body weight change in nude mice bearing human breast cancer carcinoma MT-3 xenografts treated with conjugates CDx1, CDx2, CDx3 and CDx4 vs. mice in the control group (untreated mice (saline)) as well as vs. mice treated with Doxorubicin.

The following symbols are used:

▪=saline, ★=Doxorubicin, ⋄CDx1, ∇=CDx2, ◯=CDx3, Δ=CDx4.

The X-axis shows the time after start [d], the Y-axis shows the body weight change, BWC [%].

Each measurement was carried out with a group of 8 mice. The conjugates CDx1, CDx2, CDx3 and CDx4 were administered once at a dosage of 20 mg/kg body weight on day 7. Doxorubicin was administered once at a dosage of 8 mg/kg body weight on day 7. Median values are given. Further details are given in Table 16.

### EXAMPLES

### 1. Materials and Methods

Centrifugation was performed using a Sorvall Evolution RC centrifuge (Thermo Scientific) equipped with a SLA-3000 rotor (6×400 mL vessels) at 9000 g and 4° C. for 5-10 min. Ultrafiltration was performed using a Sartoflow Slice 200 Benchtop (Sartorius AG) equipped with two Hydrosart Membrane cassettes (10 kDa Cutoff, Sartorius). Pressure settings: p1=2 bar, p2=0.5 bar.

### Filtration:

Solutions were filtered prior to size exclusion chromatography and HPLC using syringe filters (0.45 μm, GHP-Acrodisc, 13 mm) or Steriflip (0.45 μm, MilliQ).

Analytical HPLC spectra were measured on a Ultimate 3000 (Dionex) using a LPG-3000 pump, a DAD-3000a diode array detector and a C18 reverse phase column (Dr. Maisch, Reprosil Gold 300A, C18, 5 μm, 150×4.6 mm). Eluents were purified water (Millipore)+0.1% TFA (Uvasol, MERCK) and acetonitrile (HPLC grade, MERCK)+0.1% TFA. Standard gradient was: 2% ACN to 98% ACN in 30 min.

Size exclusion chromatography was performed using an Äkta Purifier (GE-Healthcare) system equipped with a P-900 pump, a P-960 sample pump using an UV-900 UV detector and a pH/IC-900 conductivity detector. A HiPrep 26/10 desalting column (53 mL, GE-Healthcare) was used together with a HiTrap desalting column as pre-column (5 mL, GE-Healthcare). Fractions were collected using the Frac-902 fraction collector.

### Freeze-Drying:

Samples were frozen in liquid nitrogen and lyophylized using a Christ alpha 1-2 LD plus (Martin Christ, Germany) at p=0.2 mbar.

UV-vis absorbances were measured at a Cary 100 BIO (Varian) in either plastic cuvettes (PMMA, d=10 mm) or quarz cuvettes (d=10 mm, Hellma, Suprasil, 100-QS) using the Cary Win UV simple reads software.

### 1.1 Reagents

TABLE 2

| | | | | |
|---|---|---|---|---|
| Entry | Name | Quality | Supplier | Lot # |

General procedure 1

| | | | | |
|---|---|---|---|---|
| 1 | 4-nitrophenyl | 96% | Aldrich | 02107CH-029 |
| | chloroformate | | | |
| 2 | Dimethyl sufoxide | dry, SeccoSolv | Merck | K39250731 |
| 3 | Pyridine | puriss | Merck | K37206362 |
| 4 | Carbohydrazid | | Acros | A0119079 |

General procedure 2

| | | | | |
|---|---|---|---|---|
| 7 | Sodium hydride | 60% w/w in paraffin | Merck | S4977752 |
| 8 | Allyl bromide | reagent grade 97% | Aldrich | S77053-109 |
| 9 | Potassium monopersulfate | technical grade | Aldrich | 82070 |
| | Triplesalt (Oxone ®) | | | |
| 10 | Sodium bicarbonate | puriss | Merck | 26533223 |
| 11 | Tetrahydrothiopyran-4-on | 99% | Aldrich | 1370210 42708159 |
| 12 | Mercaptoethanol | | | |
| 13 | Methyl chloroformiat | | Merck | S4528657 852 |

general procedure 4

| | | | | |
|---|---|---|---|---|
| 14 | 2,4,6-trinitrobenzensulfonic | 5% w/v in methanol | Sigma | S06291 |
| | acid (TNBS) | | | |
| 15 | Acetylhydrazid | 90% | Alfa | 10117464 |
| | | | Aesar | |

Solvents

| | | | | |
|---|---|---|---|---|
| 16 | Isopropanol | puriss ACS | Fluka | |
| 17 | Methyl tert. butyl ether | 99% | Acros | |
| 18 | Dimethyl formamide | pept. syn. grade | Acros | A0256931 |
| 19 | Dimethyl formamide | extra dry 99.8% | Acros | A00954967 |
| 20 | Formamide | spectophotometric | Aldrich | 59096HK |
| | | grade > 99% | | |
| 21 | Acetic acid | >99.8% | Fluka | 91190 |
| 21 | Hydrazin hydrate | | Aldrich | S55294-438 |

TABLE 3

| Entry | Name | Lot. Nr. | Mw | Mn | PD | MS |
|---|---|---|---|---|---|---|
| 1 | HES1 | 060142 | 69.7 | 51.3 | 1.36 | 1.3 |
| 2 | HES2 | 073121 | 84.5 | 55.2 | 1.52 | 1.3 |
| 3 | HES3 | 073421 | 108.1 | 89.9 | 1.20 | 0.4 |
| 4 | HES4 | 084721 | 243.9 | 183.6 | 1.33 | 1.3 |
| 5 | HES5 | 080741 | 258.9 | 237.6 | 1.32 | 1.0 |
| 6 | HES6 | 17090821 | 769.5 | 498.6 | 1.54 | 1.3 |
| 7 | HES7 | 17091331 | 985.0 | 500.0 | 1.97 | 0.5 |
| 8 | HES8 | 17090921 | 7976.0 | 3208.5 | 2.49 | 0.7 |
| 9 | HES9 | 061011 | 84.1 | 69.0 | 1.20 | 1.0 |
| 10 | HES10 | 063211 | 78.2 | 63.2 | 1.23 | 1.0 |
| 11 | HES11 | 072011 | 88.6 | 64.6 | 1.37 | 1.0 |

### 1.2 General Procedure for the Synthesis of Carbohydrazid-HES Via Activation with 4-nitrophenylchloroformiate (GP1)

### GP1.1 Activation of HES

In a 250 mL round bottom flask equipped with a magnetic stirring bar and rubber septum, HES was dissolved in a 1:1 mixture of dry DMSO and dry pyridine (to give a 25% w/w HES solution) under an atmosphere of argon. After complete dissolution of the HES (up to 2 h), the clear solution was cooled in an ice-salt bath to −10° C. (intrinsic temperature). Then, solid p-nitrophenylchloroformiate was added in portions over a 5 minute period, not allowing the temperature to rise above −5° C. The yellowish slurry was allowed to stir for additional 30 minutes at −10 to −5° C., then carefully poured into 600 mL of vigorously stirred isopropanol. The precipitated HES was collected by filtration over a sinter funnel (pore 4), washed with 3×100 mL of isopropanol followed by 3×100 mL of methyl tert.-butyl ether. The colourless solid was collected and used in the next step without further purification.

GP1.2 Reaction of Activated HES with Carbohydrazide

The activated HES was transferred into a 500 mL round bottom flask with a magnetic stirring bar and stirred for 5 minutes in water (10 mL water per gram HES starting material), forming a bright yellow suspension. Carbohydrazide was added in one portion and the resulting mixture was allowed to stir over night at ambient temperature. The resulting bright yellow mixture, which had partially dissolved, was centrifuged and the supernatant precipitated in vigorously stirred isopropanol (7-10 times the volume). The precipitated polymer was collected by centrifugation, re-dissolved in 400 mL of water and subjected to ultrafiltration (concentrated to 100 mL, then 20 volume exchanges with water). The retentate was collected and lyophilized. In order to deplete high molecular weight impurities, the crude product was further purified by fractionated precipitation.

### a) Fractionated Precipitation of the Crude Product

The crude product was dissolved in DMSO to give a 10% HES solution. Then isopropanol was added until the solution became slightly cloudy. The mixture was centrifuged, and the precipitate isolated. More isopropanol was added and the centrifugation step was repeated.

The isolated fractions were dissolved in water, freeze-dried and analyzed via GPC for their molar mass distribution. Usually the last fraction, which produces substantial amounts of material contains the non-crosslinked polymer.

### 1.3 Synthesis of D5

### a) Activation of HES with Methylchloroformiate

In a 250 mL round bottom flask equipped with a magnetic stirring bar, 10 g HES6 were dissolved in a mixture of 25 mL dry DMF and 25 mL dry pyridine under an atmosphere of argon. After the HES had completely dissolved (4 h), the clear solution was cooled in an ice bath. 1.79 mL (2.18 g) of methylchloroformiate was added dropwise over a period of 5-10 min. The reaction mixture was allowed to stir in the cooling bath for additional 90 min, then carefully poured into 350 mL vigorously stirred isopropanol. The colourless precipitate was collected by filtration over a sinter funnel (pore 4), washed 3 times with 100 mL isopropanol followed by 3 times 100 mL MTBE. The activated HES was dried for 1 h at 25 mbar to give 13.6 g of a colourless solid.

### b) Reaction of Activated HES with Carbohydrazide

In a 100 mL round bottom flask equipped with a magnetic stirring bar, 6.8 g (one half) of the crude product from the last step were dissolved in 40 mL of water. 12.25 g of carbohydrazide were added and the colourless solution (with residual undissolved carbohydrazide) heated to 70° C. (oil bath temperature). After 18 h the heating bath was removed and the mixture allowed to cool to room temperature. The mixture was poured into 280 mL of isopropanol and the precipitate collected by centrifugation. The supernatant was discarded, the residue dissolved in 100 mL of water and subjected to ultrafiltration (15 volume exchanges with water). Lyophilization afforded 4.82 g (96%) of a colourless solid. The reactive group contents were determined to be 99 nmol of hydrazine groups/mg derivate. Molecular weight distribution (determined by SEC): Mw=770 kDa, Mn=505 kDa.

### 1.4 Synthesis of D14, D15 and D21

### 1.4.1 D14 and D15

### a) Activation of HES with Methyl Chloroformate

In a 250 mL two-neck flask equipped with a magnetic stirring bar and intrinsic temperature, 10 g HES2 were dissolved in a mixture of 20 mL dry DMF and 20 mL dry pyridine under an atmosphere of argon. After the HES had completely dissolved, the clear solution was cooled to −10° C. in a dry-ice-ethanol bath. 2.43 mL of methyl chloroformate were added dropwise over a period of 5-10 min. The reaction mixture was stirred in the cooling bath for additional 60 min, then carefully poured into 600 mL vigorously stirred isopropanol. The colorless precipitate was collected by filtration over a sinter funnel (pore 4), washed 3 times with 100 mL isopropanol followed by 3 times 100 mL MTBE. The activated HES was dried to give a colorless powder.

### b) Reaction of Activated HES with Hydrazine

In a 100 mL round bottom flask equipped with a magnetic stirring bar, 7.9 g of the crude product from the last step were suspended in 20 mL of hydrazine hydrate. After 1 h, the mixture cleared up and was allowed to stir at room temperature for additional 2 h. The mixture was poured into 300 mL of isopropanol and the precipitate collected by centrifugation. The supernatant was discarded, the residue dissolved in 100 mL of water and subjected to ultrafiltration (15 volume exchanges with water). Lyophilization of the retentate afforded D14 (6.85 g, 87%) as a colourless solid. The reactive group content was determined to be 328 nmol of hydrazine groups/mg derivate. Molecular weight distribution (determined by SEC): Mw=96 kDa, Mn=67 kDa.

Derivative D15 was synthesized from 5 g HES11 and 0.5 mL methyl chloroformate in an analogue fashion to yield 4.2 g (84%) of the carbazate-functionalized HES with a molar substitution of 160 nmol/mg. Molecular weight distribution (determined by SEC): Mw=91 kDa, Mn=64 kDa.

### 1.4.2 D21

### a) Reaction of HES with Methyl Bromoacetate

In a 500 mL round bottom flask, 22.2 g of HES2, previously dried for several hours at 80° C., was dissolved in 200 mL of DMF (peptide synthesis grade) under an atmosphere of argon. After addition of 2 g of sodium hydride (60% in paraffin), the reaction was allowed to stir over night to give a yellow, jelly-like mixture. Methyl bromoacetate (2.32 mL) was added and the mixture reacted for 4 h at room temperature turning the jelly-like mixture into a light yellow suspension. The reaction was quenched by addition of 4.62 ml, of acetic acid and the mixture precipitated into 1.4 L of isopropanol. The polymer was collected by centrifugation and re-dissolved in 200 mL of water. The polymer was purified by ultrafiltration (15 volume exchanges against water) and freeze-dried to yield 16.4 g (74%) of a light yellow solid.

### b) Preparation of Title Compound

15 g of the product of the previous step were weighed into a 500 mL round bottom flask and dissolved in 150 mL of hydrazine monohydrate (64% hydrazine content). The resulting clear, light yellow solution was allowed to stir for 3.5 h, then poured into 1.1 L of isopropanol. The supernatant was discarded and the residue re-dissolved in 160 mL of water. The polymer was purified by dialysis against 5×10 L of water (regenerated cellulose, 10 kDa cutoff), the retentate filtered and freeze-dried to yield 11.1 g (74%) of D22 as a colorless solid.

### 1.5 General Procedure for the Synthesis of Multi-Carbohydrazide HES Via Multi-Epoxy HES (GP2)

### General Procedure for the Synthesis of Multi-Allyl HES (2.1)

Hydroxyethyl starch used in the preparation was thoughtfully dried prior to use either on an infra-red heated balance at 80° C. until the mass remained constant or by leaving in a drying oven over night at 80° C. A 10% solution of the dry HES in dry DMF or formamide (photochemical grade) was prepared in a round bottom flask equipped with a magnetic stirring bar and a rubber septum under an atmosphere of inert gas. Sodium hydride (60% w/w in paraffin) was added in one portion and the resulting cloudy solution was allowed to stir for 1 h at room temperature followed by the addition of allyl bromide. The reaction mixture was allowed to stir over night, resulting in a colorless-light brown, clear solution. The solution was then slowly poured into 7-10 times the volume of isopropanol and the precipitate collected by centrifugation. The precipitated polymer was re-dissolved in water and subjected to ultrafiltration (15-20 volume exchanges with water). Freeze-drying of the retentate yielded a colorless solid.

### General Procedure for the Synthesis of Multi-Epoxy HES (2.2)

In a glass beaker, multi-allyl-HES was dissolved in a 4*10⁻⁴ M EDTA solution (10-15 mL/g HES). Tetrahydrothiopyran-4-on was added and the solution stirred on a magnetic stirring plate. Potassium peroxymonosulfate (Oxone®) and sodium hydrogen carbonate were mixed in dry state and the mixture added in small portions to the HES-solution resulting in formation of thick foam. The mixture was stirred at ambient temperatures for 2 h, diluted with water to a volume of 100 mL and then directly purified by ultrafiltration (15-20 volume exchanges with water). The resulting retentat was collected and directly used in the next step.

General Procedure for the Nucleophilic Ring Opening of Multi-Epoxy HES with Carbohydrazide (2.3)

The aqueous solution of epoxydized HES from GP2.2 was transferred into a reaction vessel containing a magnetic stirring bar and carbohydrazide or another divalent linker molecule. After 1-5 days of stirring at the specified temperature, the desired HES derivative was purified by ultrafiltration (in case of a sample >1 g, 15-20 volume exchanges with water) or size exclusion chromatography (for samples ≦1 g). The pure product was obtained by lyophilization of either the retentate of the ultrafiltration or the polymeric fractions of the chromatography.

### 1.7 General Procedure for the Determination of Reactive Group Content of HES-Carbohydrazid Derivatives with 2,4,6-trinitrobenzolsulfonic acid (TNBS) (GP4)

The TNBS reagent (2,4,6-trinitrobenzosulfonic acid, picrylsulfonic acid) was used as a 5% (w/v) solution in methanol, which was further diluted (177 μL TNBS solution+823 mL 0.1 M borate buffer pH9.3) to give stock solution A (0.03 mol/L).

Acetyl hydrazide was used as calibration for hydrazide functionalization. Six standard solutions of acetyl hydrazide in 0.1 M borate buffer (pH 9.3) were prepared ranging from ˜7 to ˜80 nmol/mL.

1 mg/mL solutions of the HES samples in 0.1 M borate buffer (pH 9.3) were prepared and further diluted with buffer to give 1 mL samples containing 0.3 mg/mL polymer.

A sample of 1 mL buffer was used as a blank.

Both blank, HES-solutions and the standard solutions were mixed with 25 μL of stock solution A and incubated at 40° C. in a thermomixer (750 rpm). The yellow-orange solutions were transferred into plastic cuvettes (d=1 cm) and their absorbance measured at 500 nm (minus the blank). The absorbance of the six standards was plotted against their concentration, resulting in a linear equation with the slope resembling the extinction coefficient (ranging from 12.50 cm²/μmol to 13.50 cm²/μmol). This linear equation was used to calculate the molar concentration of hydrazide groups in the polymer sample in μmol/mL and converted into nmol hydrazide/mg derivate. Loading values given in the experimentals are the mean value of three independent measurements.

### 1.8 General Procedure for the Determination of the Drug Content (GP4)

1 ml of a 0.5 mg/ml solution of a HES-drug conjugate in the appropriate solvent was measured at the absorbance maximum (see table 4a) (at 480 nm for doxorubicin) in a plastic cuvette (d=1 cm) using pure water as blank. The absorption of the blank was substracted from the conjugate and the drug content calculated as follows:

$${c}_{drug} [ μmol \text{/} mL ] = \frac{( {A}_{480} - {A}_{480}^{0} )}{10.925 \frac{{cm}^{2}}{μmol} * 1 cm}$$

The molar extinction coefficients were obtained from a calibration curve of the drugs (as hydrochlorides) in the specific solvents at the appropriate wavelength.

The loading is calculated as

$$Loading [ μmol \text{/} g ] = \frac{1000 * {c}_{drug} [ μmol \text{/} mL ]}{{c}_{conjugate} [ mg \text{/} mL ]}$$

with c_{conjugate} being the concentration of the sample solution, e.g. 0.5 mg/ml.

With a known molecular weight for the drug (e.g. 579.98 g/mol for doxorubicin), the drug loading can also be expressed in mg drug/gram conjugate:

Loading[mg/g]=Loading[μmol/g]*M_{w}[g/mol]/1000

TABLE 4
Extinction coefficients determined from calibration curves in H₂O

| # | Drug | Solvent | Wavelength [nm] | ε[cm²/μmol] | M_{w} [g/mol] |
|---|---|---|---|---|---|
| 1 | Doxorubicin | H₂O | 480 | 10.925 | 579.98 |
| 2 | Epirubicin | H₂O | 480 | 10.443 | 579.98 |
| 3 | Idarubicin | H₂O | 480 | 9.056 | 534 |

### 1.9 General Procedure for the Determination of the Mean Molecular Weight MW

The “mean molecular weight” as used in the context of the present invention relates to the weight as determined according to MALLS-GPC (Multiple Angle Laser Light Scattering-Gel Permeation Chromatography).

For the determination, 2 Tosoh BioSep GMPWXL columns connected in line (13 μm particle size, diameter 7.8 mm, length 30 cm, Art. no. 08025) were used as stationary phase. The mobile phase was prepared as follows: In a volumetric flask 3.74 g Na-Acetate*3H₂O, 0.344 g NaN₃ are dissolved in 800 ml Milli-Q water and 6.9 ml acetic acid anhydride are added and the flask filled up to 11.

Approximately 10 mg of the hydroxyalkyl starch derivative were dissolved in 1 ml of the mobile phase and particle filtrated with a syringe filter (0.22 mm, mStarII, CoStar Cambridge, Mass.)

The measurement was carried out at a Flow rate of 0.5 ml/min.

As detectors a multiple-angle laser light scattering detector and a refractometer maintained at a constant temperature, connected in series, were used.

Astra software (Vers. 5.3.4.14, Wyatt Technology Cooperation) was used to determine the mean M_{w} and the mean Mₙ of the sample using a dn/dc of 0.147. The value was determined at λ=690 nm (solvent NaOAc/H₂O/0.02% NaN₃, T=20° C.) in accordance to the following literature: W. M. Kulicke, U. Kaiser, D. Schwengers, R. Lemmes, Starch, Vol. 43, Issue 10 (1991), 392-396.

TABLE 1
Synthesis and characterization of CH-HES derivatives according to GP1

| | HES | | pNCF | CH^{a} | Frac. Prec.^{b} | Yield | Loading | Mw | Mn |
|---|---|---|---|---|---|---|---|---|---|
| # | | m[g] | m[g] | m[g] | V_{DMSO}/V_{IPA} | [%] | [nmol/mg] | [kD] | [kD] |
| D1 | HES2 | 20 | 9.23 | 15.23 | 90/104 | 68 | 202 | 328 | 89 |
| D2 | HES4 | 10 | 4.64 | 7.62 | 93/130 | 35 | 116 | 229 | 171 |
| D3 | HES5 | 20 | 9.28 | 11.50 | 60/62 | 32 | 150 | 289 | 209 |
| D4 | HES6 | 5 | 2.30 | 10.36 | 32/40 | 20 | 148 | 1956 | 724 |

^{a}Carbohydrazide
^{b}Ratio of DMSO/isopropanol, when high molecular weight impurities precipitated from the solution.
Starting point is a 10% (w/v) solution of crude derivative in DMSO.
Further addition in isopropanol led to precipitation of the title derivative with the characteristics specified in the table.

TABLE 2
Synthesis and characterization of Allyl-
HES intermediates according to GP2.1

GP2.1

| | | | |
|---|---|---|---|
| HES | NaH | AllBr | Yield |

| # | | m[g] | m[g] | V[mL] | Solv. | [%] |
|---|---|---|---|---|---|---|
| I1 | HES2 | 5.00 | 0.20 | 0.58 | DMF | n.d. |
| I2 | HES4 | 10.00 | 0.81 | 1.17 | DMF | 93 |
| I3 | HES6 | 5.00 | 0.27 | 0.47 | DMF | 90 |
| I4 | HES1 | 5.00 | 0.20 | 0.35 | DMF | 96 |
| I5 | HES3 | 5.00 | 0.42 | 0.72 | FA | 89 |
| I6 | HES7 | 3.00 | 0.15 | 0.35 | FA | 84 |
| I7 | HES8 | 5.00 | 0.23 | 0.40 | FA | 96 |
| I8 | HES2 | 10.00 | 0.14 | 0.23 | DMF | 98 |
| I9 | HES9 | 10.00 | 0.29 | 0.50 | DMF | 94 |
| I10 | HES10 | 10.00 | 0.29 | 0.50 | DMF | 95 |
| I11 | HES11 | 10.00 | 0.29 | 0.50 | DMF | 98 |

TABLE 3
Synthesis and characterization of HES-CHHP-derivatives according to GP2.2 and GP2.3

| | | | GP2.2 | | | GP2.3 | | | | | | |
|---|---|---|---|---|---|---|---|---|---|---|---|---|
| | Allyl HES | | Oxone ® | NaHCO₃ | THTP | | | t/T | Yield | Loading | Mw | Mn |
| # | | m[g] | m[g] | m[g] | m[mg] | Nuc^{a} | m[g] | [d]/° C. | [%] | nmol/mg | [kD] | [kD] |
| D6 | I1 | | 6.28 | 2.68 | 39 | CH | 3.05^{b} | 5/RT | 79 | 308 | 103 | 74 |
| D7 | I2 | 5.00 | 12.50 | 5.29 | 81 | CH | 12.20 | 3/RT | 40 | 444 | 343 | 254 |
| D8 | I3 | 4.51 | 5.38 | 2.49 | 38 | CH | 12.00 | 3/RT | 90 | 211 | 1033 | 575 |
| D9 | I4 | 4.81 | 3.61 | 1.53 | 25 | CH | 7.05 | 4^{c}/RT | 99 | 329 | 71 | 55 |
| D10 | I5 | 4.45 | 6.82 | 2.90 | 44 | CH | 13.36 | 4^{c}/RT | 99 | 387 | 109 | 88 |
| D11 | I6 | 2.50 | 2.24 | 0.95 | 15 | CH | 8.75 | 2^{c}/RT | 84 | 229 | 960 | 553 |
| D12 | I7 | 2.50 | 2.14 | 0.91 | 14 | CH | 8.35 | 4/RT | 96 | 236 | n.d. | n.d. |
| D13 | I8 | 5.00 | 1.25 | 0.55 | 16 | CH | 2.44^{b} | 2.5/RT | 85 | 128 | 89 | 61 |
| D16 | I11 | 5.00 | 5.33 | 2.26 | 46 | CH | 2.61^{d} | 2.5/30° C. | 86 | 257 | 98 | 69 |
| D17 | I11 | 4.75 | 7.81 | 1.10 | 49 | CH | 5.09 | 1/40° C. | 96 | 187 | 113 | 75 |
| D18 | I10 | 7.00 | 7.46 | 3.16 | 51 | SDH | 11.0 | 2/50° C. | 90 | 250 | 99 | 70 |
| D19 | I9 | 3.00 | 3.20 | 1.35 | 20 | SDH | 5.0 | 2.5/50° C. | 86 | 273 | 99 | 71 |
| D20 | I9 | 6.00 | 6.39 | 2.71 | 42 | ADH | 1.51^{e} | 2.5/40° C. | 82 | 307 | 99 | 76 |

^{a}Nucleophile: CH = carbohydrazide, ADH = adipic dihydrazide, SDH = succinic dihydrazide,
^{b}The retentate of the ultrafiltration (GP2.2) was divided into two aliquots. Amount of carbohydrazide refers to ½ of the amount of allyl-HES.
^{c}After the reaction time, 2 mL mercaptoethanol were added and the mixture stirred for 2 h prior to ultrafiltration.
^{d}The retentate of the ultrafiltration (GP2.2) was divided into four aliquots. Amount of carbohydrazide refers to 1.25 g of epoxidated allyl-HES.
^{e}The retentate of the ultrafiltration (GP2.2) was divided into six aliquots. Amount of HA refers to 1 g of epoxidated allyl-HES. Purification by SEC.

TABLE 7
Synthesis and characterization of HES-anthracycline conjugates according to GP3.

| | | | | Anthracycline | Reaction | Dilution | | | | | |
|---|---|---|---|---|---|---|---|---|---|---|---|
| | Derivative | | Buffer | hydrochloride | time | H₂O | Yield^{a} | Purity | Loading | Mw | Mn |
| # | | m[g] | V [mL] | m[g] | [h] | V [mL] | [%] | [%] | [mg Dx/g] | [kD] | [kD] |
| CDx1 | D1 | 2.50 | 46 | 1.465 | 48 | 31 | 90 | 99.7 | 87.4 | 125 | 87 |
| CDx2 | D2 | 2.50 | 46 | 1.204 | 72 | 31 | 80 | 99.9 | 45.1 | 259 | 197 |
| CDx3 | D3 | 2.50 | 46 | 1.088 | 60 | 31 | 74 | 99.9 | 69.4 | 332 | 229 |
| CDx4 | D4 | 1.03 | 17 | 0.443 | 100 | 13 | n.d. | n.d. | 66.2 | 1167 | 662 |
| CDx5 | D5 | 1.02 | 20 | 0.289 | 92 | 12 | 50 | >99.9 | 33.0 | 942 | 552 |
| CDx6 | D6 | 0.80 | 15 | 0.718 | 72 | 10 | 56 | 99.5 | 101.0 | 145 | 108 |
| CDx7 | D7 | 0.50 | 10 | 0.132 | 68 | 10 | 61 | 97.1 | 121.9 | 514 | 367 |
| CDx8 | D8 | 1.00 | 17 | 0.612 | 43 | 11 | 59 | 99.8 | 82.8 | 1079 | 575 |
| CDx9 | D8 | 1.00 | 20 | 0.122 | 48 | 13 | 79 | 99.8 | 44.9 | 1015 | 508 |
| CDx10 | D9 | 1.00 | 20 | 0.192 | 40 | 13 | 75 | >99.9 | 76.1 | 96 | 70 |
| CDx11 | D10 | 1.01 | 20 | 0.212 | 40 | 13 | 58 | 98.3 | 98.0 | 154 | 120 |
| CDx12 | D11 | 1.00 | 20 | 0.665 | 44 | 20 | 33 | 99.7 | 79.5 | 1740 | 741 |
| CDx13 | D12 | 1.00 | 20 | 0.684 | 41 | 30 | 31 | 99.9 | 66.3 | 819 | 616 |
| CDx14 | D13 | 1.00 | 20 | 0.298 | 68 | 8 | 67 | >99.9 | 35.5 | 102 | 70 |
| CDx15 | D14 | 0.75 | 14 | 0.713 | 48 | 9 | 70 | 98.8 | 92.6 | 135 | 95 |
| CDx16 | D17 | 0.5 | 10 | 0.217 | 24 | 20 | 88 | 99.6 | 58.6 | 150 | 86 |
| CDx17 | D19 | 0.5 | 7.5 | 0.317 | 16 | 22.5 | 79 | 99.1 | 36.7 | 103 | 74 |
| CDx18 | D18 | 1.0 | 15 | 0.58 | 48 | 30^{b} | 84 | 98.2 | 39.2 | 102 | 73 |
| CDx19 | D21 | 0.5 | 7.5 | 0.312 | 60 | 22.5^{b} | 75 | 96.3 | 38.8 | 95 | 57 |
| CDx20 | D20 | 0.6 | 9 | 0.427 | 16 | 19 | 86 | 97.1 | 38.1 | n.d. | n.d. |
| CEp2 | D19 | 0.5 | 7.5 | 0.317 | 16 | 22.5 | 83 | 99.4 | 35.6 | 103 | 74 |
| CEp3 | D18 | 1.0 | 15 | 0.58 | 16 | 30^{b} | 92 | 99.4 | 37.1 | 105 | 73 |
| CId1 | D17 | 0.5 | 10 | 0.105 | 16 | 20 | 83 | 99.5 | 72.8 | 101 | 55 |
| CId2 | D15 | 0.5 | 10 | 0.085 | 16 | 20 | 87 | 99.8 | 68.8 | 109 | 76 |
| CId3 | D19 | 0.5 | 7.5 | 0.146 | 16 | 12.5 | 88 | 99.3 | 61.5 | 105 | 77 |

^{a}Calculated as [100*m_{conjugate}]/[m_{derivative}*(1 + Loading/1000)]
^{b}Reaction diluted with pept. synthesis grade DMF instead of water

TABLE 8
Overview of the synthesized hydroxyethyl starch derivatives

| | | Structure of HES derivative |
|---|---|---|
| | | 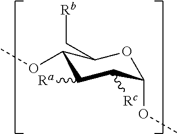 |
| | | with at least one of R^{a}, R^{b} or R^{c} of the shown structural unit being: |
| | | —[O—CH₂—CH₂]ₜ—[F¹]ₚ—[L^{t}]_{q}—[F²]ᵣ—[L²]ᵥ—Z¹ |
| | | wherein t is 0-4, |
| | | and wherein |
| | HES | —[F¹]ₚ—[L^{t}]_{q}—[F²]ᵣ—[L²]ᵥ—Z¹ |
| Code | used | is: |
| D1 | HES 2 | —O—C(═O)—NH—NH—C(═O)—NH—NH₂ |
| D2 | HES 4 | —O—C(═O)—NH—NH—C(═O)—NH—NH₂ |
| D3 | HES 5 | —O—C(═O)—NH—NH—C(═O)—NH—NH₂ |
| D4 | HES 6 | —O—C(═O)—NH—NH—C(═O)—NH—NH₂ |
| D5 | HES 6 | —O—C(═O)—NH—NH—C(═O)—NH—NH₂ |
| D6 | HES 2 | —O—C(═O)—NH—NH—C(═O)—NH—NH₂ |
| D7 | HES 4 | —O—CH₂—CHOH—CH₂—NH—NH—C(═O)—NH—NH₂ |
| D8 | HES 6 | —O—CH₂—CHOH—CH₂—NH—NH—C(═O)—NH—NH₂ |
| D9 | HES 1 | —O—CH₂—CHOH—CH₂—NH—NH—C(═O)—NH—NH₂ |
| D10 | HES 3 | —O—CH₂—CHOH—CH₂—NH—NH—C(═O)—NH—NH₂ |
| D11 | HES 7 | —O—CH₂—CHOH—CH₂—NH—NH—C(═O)—NH—NH₂ |
| D12 | HES 8 | —O—CH₂—CHOH—CH₂—NH—NH—C(═O)—NH—NH₂ |
| D13 | HES 2 | —O—CH₂—CHOH—CH₂—NH—NH—C(═O)—NH—NH₂ |
| D14 | HES 2 | —O—C(═O)—NH—NH₂ |
| D15 | HES 11 | —O—C(═O)—NH—NH₂ |
| D16 | HES 11 | —O—CH₂—CHOH—CH₂—NH—NH—C(═O)—NH—NH₂ |
| D17 | HES 11 | —O—CH₂—CHOH—CH₂—NH—NH—C(═O)—NH—NH₂ |
| D18 | HES 10 | —O—CH₂—CHOH—CH₂—NH—NH—C(═O)—CH₂—CH₂—C(═O)—NH—NH₂ |
| D19 | HES 9 | —O—CH₂—CHOH—CH₂—NH—NH—C(═O)—CH₂—CH₂—C(═O)—NH—NH₂ |
| D20 | HES 9 | —O—CH₂—CHOH—CH₂—NH—NH—C(═O)—(CH₂)₄—C(═O)—NH—NH₂ |
| D21 | HES 2 | —O—CH₂—C(═O)—NH—NH₂ |

TABLE 9a
Overview of the synthesized hydroxyethyl starch doxorubicin conjugates

| | | Structure of HES derivative |
|---|---|---|
| | | 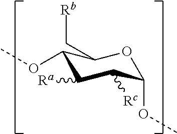 |
| | | 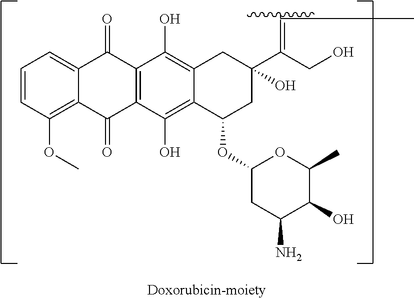 |
| | | with at least one of R^{a}, R^{b} or R^{c} of the shown structural unit being: |
| | | —[O—CH₂—CH₂]ₜ—[F¹]ₚ—[L¹]_{q}—[F²]ᵣ—[L²]ᵥ—X═] = Doxorubicin-residue |
| | | wherein t is 0-4, |
| | | and wherein |
| | HES | —[F¹]ₚ—[L¹]_{q}—[F²]ᵣ—[L²]ᵥ—X═] |
| Code | used | is: |
| CDx1 | HES 2 | [—O—C(═O)—NH—NH—C(═O)—NH—N═] |
| CDx2 | HES 4 | [—O—C(═O)—NH—NH—C(═O)—NH—N═] |
| CDx3 | HES 5 | [—O—C(═O)—NH—NH—C(═O)—NH—N═] |
| CDx4 | HES 6 | [—O—C(═O)—NH—NH—C(═O)—NH—N═] |
| CDx5 | HES 6 | [—O—C(═O)—NH—NH—C(═O)—NH—N═] |
| CDx6 | HES 2 | [—O—C(═O)—NH—NH—C(═O)—NH—N═] |
| CDx7 | HES 4 | [—O—CH₂—CHOH—CH₂—NH—NH—C(═O)—NH—N═] |
| CDx8 | HES 6 | [—O—CH₂—CHOH—CH₂—NH—NH—C(═O)—NH—N═] |
| CDx9 | HES 6 | [—O—CH₂—CHOH—CH₂—NH—NH—C(═O)—NH—N═] |
| CDx10 | HES 1 | [—O—CH₂—CHOH—CH₂—NH—NH—C(═O)—NH—N═] |
| CDx11 | HES 3 | [—O—CH₂—CHOH—CH₂—NH—NH—C(═O)—NH—N═] |
| CDx12 | HES 7 | [—O—CH₂—CHOH—CH₂—NH—NH—C(═O)—NH—N═] |
| CDx13 | HES 8 | [—O—CH₂—CHOH—CH₂—NH—NH—C(═O)—NH—H═] |
| CDx14 | HES 2 | [—O—CH₂—CHOH—CH₂—NH—NH—C(═O)—NH—N═] |
| CDx15 | HES 2 | [—O—C(═O)—NH—N═] |
| CDx16 | HES 11 | [—O—CH₂—CHOH—CH₂—NH—NH—C(═O)—NH—N═] |
| CDx17 | HES 9 | [—O—CH₂—CHOH—CH₂—NH—NH—C(═O)—(CH₂)₂—C(═O)—NH—N═] |
| CDx18 | HES 10 | [—O—CH₂—CHOH—CH₂—NH—NH—C(═O)—(CH₂)₂—C(═O)—NH—N═] |
| CDx19 | HES 2 | [—CH₂—C(═O)—NH—N═] |
| CDx20 | HES 9 | [—O—CH₂—CHOH—CH₂—NH—NH—C(═O)—(CH₂)₄—C(═O)—NH—N═] |

TABLE 9b
Overview of the synthesized hydroxyethyl starch epirubicin conjugates

| | | Structure of HES derivative |
|---|---|---|
| | | 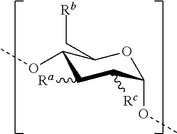 |
| | | 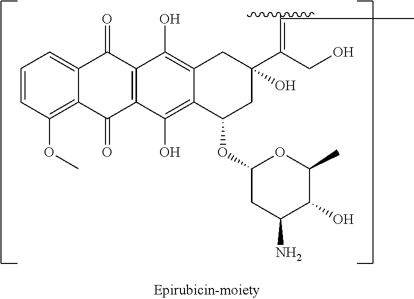 |
| | | with at least one of R^{a}, R^{b} or R^{c} of the shown structural unit being: |
| | | —[O—CH₂—CH₂]ₜ—[F¹]ₚ—[L_{q}]—[F²]ᵣ—[L²]ᵥ—X═] = Epirubicin-residue |
| | HES | wherein t is 0-4, and wherein —[F¹]ₚ—[L¹]_{q}—[F²]ᵣ—[L²]ᵥ—X═] |
| Code | used | is: |
| CEp1 | HES 11 | [—O—CH₂—CHOH—CH₂—NH—NH—C(═O)—NH—N═] |
| CEp2 | HES 9 | [—O—CH₂—CHOH—CH₂—NH—NH—C(═O)—(CH₂)₂—C(═O)—NH—N═] |
| CEp3 | HES 10 | [—O—CH₂—CHOH—CH₂—NH—NH—C(═O)—(CH₂)₂—C(═O)—NH—N═] |

TABLE 9c
Overview of the synthesized hydroxyethyl starch idarubicin conjugates

| | | Structure of HES derivative |
|---|---|---|
| | | 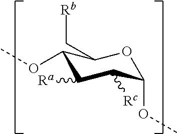 |
| | | 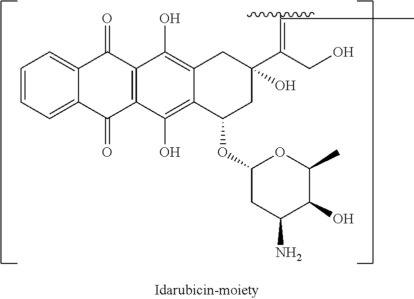 |
| | | with at least one of R^{a}, R^{b} or R^{c} of the shown structural unit being: |
| | | —[O—CH₂—CH₂]ₜ—[F¹]ₚ—[L¹]_{q}—[F²]ᵣ—[L²]ᵥ—X═] = Idarubicin-residue |
| | HES | wherein t is 0-4, and wherein —[F¹]ₚ—[L¹]_{q}—[F²]ᵣ—[L²]ᵥ—X═] |
| Code | used | is: |
| CId1 | HES 11 | [—O—CH₂—CHOH—CH₂—NH—NH—C(═O)—NH—N═] |
| CId2 | HES 11 | [—O—C(═O)—NH—N═] |
| CId3 | HES 9 | [—O—CH₂—CHOH—CH₂—NH—NH—C(═O)—(CH₂)₂—C(═O)—NH—N═] |

### 2 In Vivo Testing

### 2.1 Doxorubicin Studies

### 2.1.1 Test Animals

For all in vivo studies in which doxorubicin conjugates wer administred, adult female NMRI:nu/nu mice (TACONIC Europe, Lille Skensved, Denmark) were used throughout the study. At the start of experiment they were 6-8 weeks of age and had a median body weight of 15.4 to 26.4.

All mice were maintained under strictly controlled and standardized barrier conditions. They were housed—maximum five mice/cage—in individually ventilated cages (Macrolon Typ-II, system Techniplast, Italy). The mice were held under standardized environmental conditions: 22±1° C. room temperature, 50±10% relative humidity, 12 hour-light-dark-rhythm. They received autoclaved food and bedding (Ssniff, Soest, Germany) and acidified (pH 4.0) drinking water ad libitum.

Animals were randomly assigned to experimental groups with 7 to 8 mice each. At treatment initiation the ears of the animals were marked and each cage was labeled with the cage number, study number and animal number per cage.

Table 16 provides an overview of the animal conditions.

TABLE 16
Summary of animal conditions

| Subject | Conditions |
|---|---|
| Animals, gender | female NMRI:nu/nu mice |
| and strain | |
| Age | 6-8 weeks |
| Body weight | 15.4 to 26.4 g at the start of treatment |
| Supplier | EPO Experimental Pharmacology and Oncology, |
| | Berlin-Buch GmbH |
| Environmental | Strictly controlled and standardised barrier conditions, |
| | IVC |
| Conditions | System Techniplast DCC |
| | (TECNIPLAST DEUTSCHLAND GMBH, |
| | Hohenpeiβenberg) |
| Caging | Macrolon Type-II wire-mesh bottom |
| Feed type | Ssniff NM, Soest, Germany |
| Drinking water | autoclaved tap water in water bottles (acidified to ph 4 |
| | with HCl) |
| Feeding and | ad libitum 24 hours per day |
| drinking time | |
| Room temperature | 22 ± 1° C. |
| Relative humidity | 50 ± 10% |
| Light period | artificial; 12-hours dark/12 hours light rhythm (light |
| | 06.00 to 18.00 hours) |
| Health control | The health of the mice was examined at the start of the |
| | experiment and twice per day during the experiment. |
| Identification | Ear mark and cage labels |
| Tumor model | MT-3 (human breast cancer carcinoma); ovcar-3 |
| | (human ovarian cancer carcinoma); MT3-ADR (human |
| | mamma carcinoma). |

### 2.2 Tumor Model

The human breast cancer carcinoma MT-3, human ovarian cancer carcinoma ovcar-3 and human mamma carcinoma MT3-ADR were used as s.c. xenotransplantation model in immunodeficient female NA/RI:nu/nu mice (see table 17).

TABLE 17
Overview of the tumor models used in studies

| Name | tumor model | ATCC number | described in |
|---|---|---|---|
| MT-3 | human breast cancer | | Naunhof H. et al. |
| | | | Breast Cancer Res |
| | | | Treat. 87-95 (1992) |
| Ovcar-3 | human ovarian | HTB-161 | Hamilton TC, et al. |
| | cancer | | Cancer Res. 43: |
| | | | 5379-5389 (1983). |
| MT3-ADR | human mamma | | Stein et al. Int. J. |
| | carcinoma | | Cancer 72: 885-91 |
| | | | (1997) |

The cells were obtained from ATCC and are cryo-preserved within the EPO tumor bank. They were thawed, expanded in vitro and transplanted as cell suspension subcutaneously (s.c.) in female NMRI:nu/nu mice. The tumor lines MT-3, ovcar-3 and MT3-ADR are used for testing new anticancer drugs or novel therapeutic strategies. It was therefore selected for this study. MT-3, ovcar-3 and MT3-ADR xenografts are growing relatively fast and uniform.

### Experimental Procedure

For experimental use 5*10⁶ to 10⁷ tumor cells/mouse from the in vitro passage were transplanted s.c. into the flank of each of 10 mice/group at day 0.

### Treatment

At palpable tumor size (30-100 mm³) treatment started. The application volume was 0.2 ml/20 g mouse body weight. The test compounds, the vehicle controls and the reference compounds were all given intravenously (i.v.).

### 2.1.2 Therapeutic Evaluation

Tumor growth inhibition was used as therapeutic parameter. Additionally, body weight change was determined as signs for toxicity (particularly, potential hematological or gastrointestinal side effects).

### Tumor Measurement

Tumor diameters were measured twice weekly with a caliper. Tumor volumes were calculated according to V=(length×(width)²)/2. For calculation of the relative tumor volume (RTV) the tumor volumes at each measurement day were related to the day of first treatment. At each measurement day the median and mean tumor volumes per group and also the treated to control (T/C) values in percent were calculated (Table 10 to 15).

### Body Weight

Individual body weights of mice were determined twice weekly and mean body weight per group was related to the initial value in percent (body weight change, BWC).

### End of Experiment

On the day of necropsy the mice were sacrificed by cervical dislocation and inspected for gross organ changes.

### Statistics

Descriptive statistics were performed on the data of body weight and tumor volume. These data are reported in tables as median values, means and standard deviations, see Tables in appendix. Statistical evaluation was performed with the U-test of Mann and Whitney with a significance level of p≦0.05, using the Windows program STATISTICA 6.

### 2.1.3 Analysis of the Effects of Doxorubicin Conjugates on Tumor Growth and Body Weight

### 2.1.3 Tested Doxorubicin Conjugates Substances

The tested Doxorubicin conjugates CDx1 to CDx15 were obtained as described herein above and were kept in a freeze-dried form at −20° C. until the use. Before administration, the conjugates were dissolved in saline solution by vortexing in combination with centrifugation until a clear solution of the necessary concentration of the drug was obtained. The obtained solutions were prepared and injected under sterile conditions.

The reference compound was Doxorubicin (which is, for example, available as Doxorubicin NC® from Neocorp). Doxorubicin was stored in aliquots at 4° C. in the dark and diluted in saline before administration.

As a further control, saline solution was intravenously administered.

The above mentioned conjugates were tested in the MT3 tumor model (breast cancer). Two conjugates (CDx6 and CDx8) were additionally tested in the ovcar-3 (ovarian cancer) and the MT3-ADR (mamma carcinoma) model.

The following table provides an overview on the dosage scheme for the tested substances. Usually, the Doxorubicin conjugates were administered only once at a dosage of 20 mg/kg body weight. Besides, the conjugate CDx1 was also administered twice at a dosage of either 8 or 20 mg/kg body weight. Usually, the reference compound Doxorubicin was administered once at a dosage of 8 mg/kg. A more comprehensive overview on the dosage scheme can be found in table 10-15.

TABLE 18
Treatment groups

| | | Dose | | Doses |
|---|---|---|---|---|
| Mice | | mg/kg body | | [application × |
| [n] | Substances | weight/appl | Route | mg/kg] |
| 7-8 | Saline | — | i.v. | — |
| 7-8 | Doxorubicin | 8 | i.v. | 1 × 8 |
| 7-8 | Doxorubicin | 20 | i.v. | 1 × 20* |
| | conjugate | | | |

*amount of Doxorubicin present in the conjugate

### 2.1.4 Test Results for Doxorubicin Conjugates Substances

Tables 10 to 15 summarize the results for the tested, Doxorubicin conjugates and the reference compound Doxorubicin. The table shows, inter alia,
i) the tested compounds,
ii) the tumor volume in mice at the day the control group was sacrificed (in cm³),
iii) the lowest value of the relative tumor volume vs. the relative tumor volume of the control group (RTV T/C) together with the day, when this optimum was reached,
iv) the maximum body weight loss in mice together with the day, when this minimum was reached.

The loss of body weight is known to be an indicator of gastro-intestinal and hepatotoxicity of the tested compound.

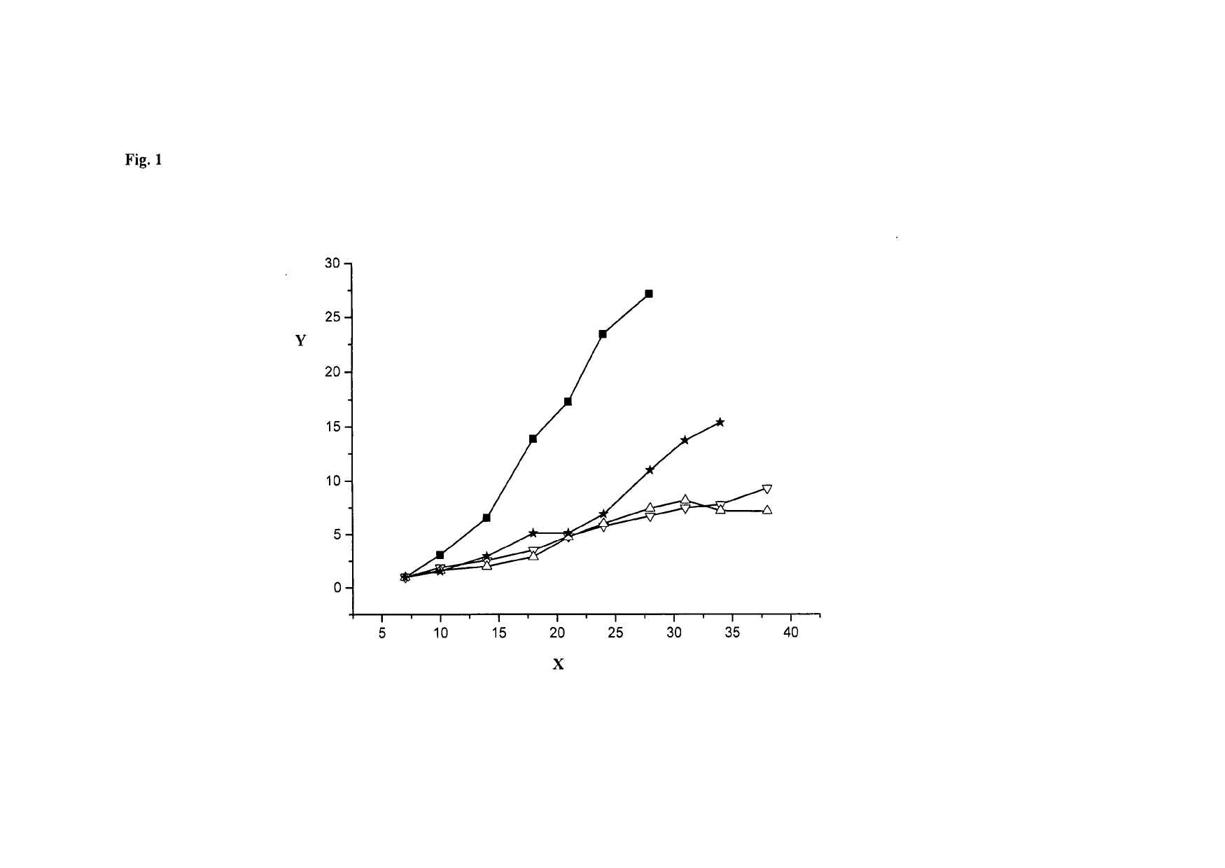

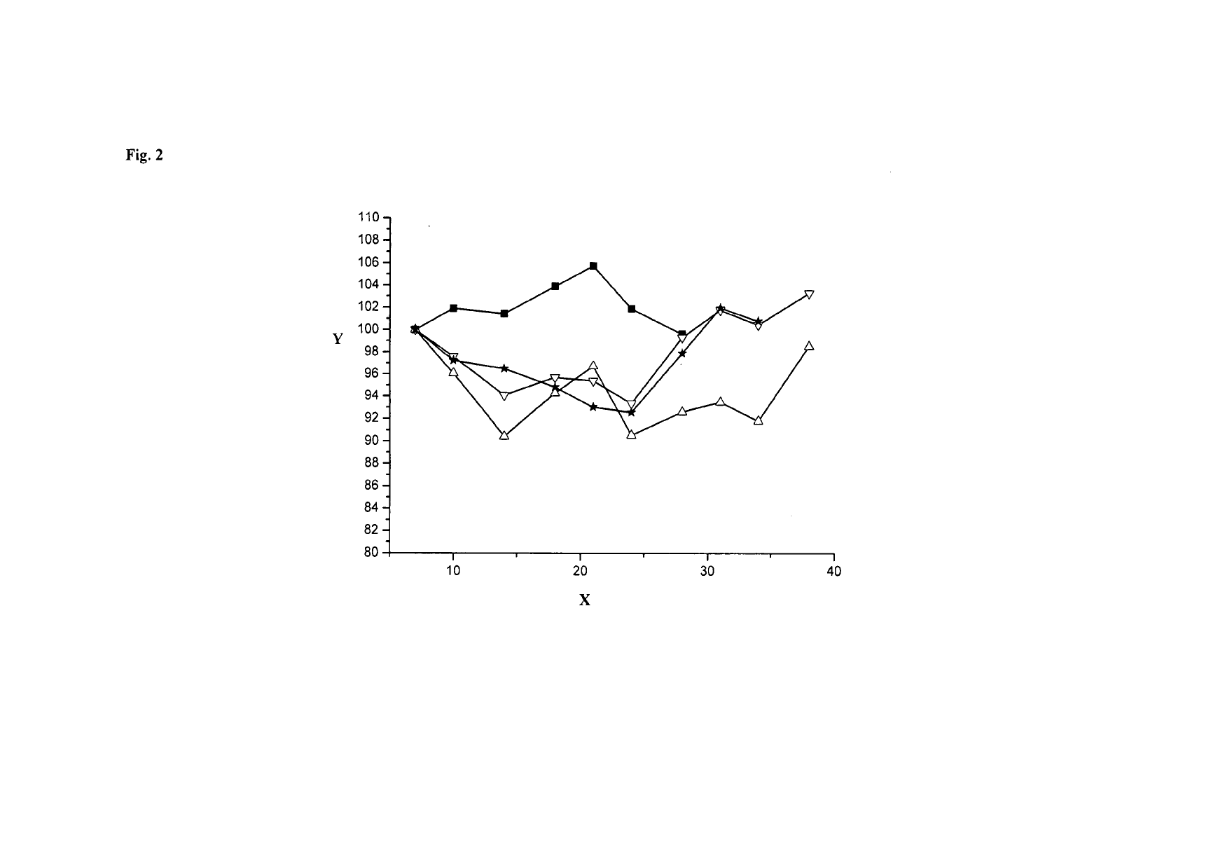

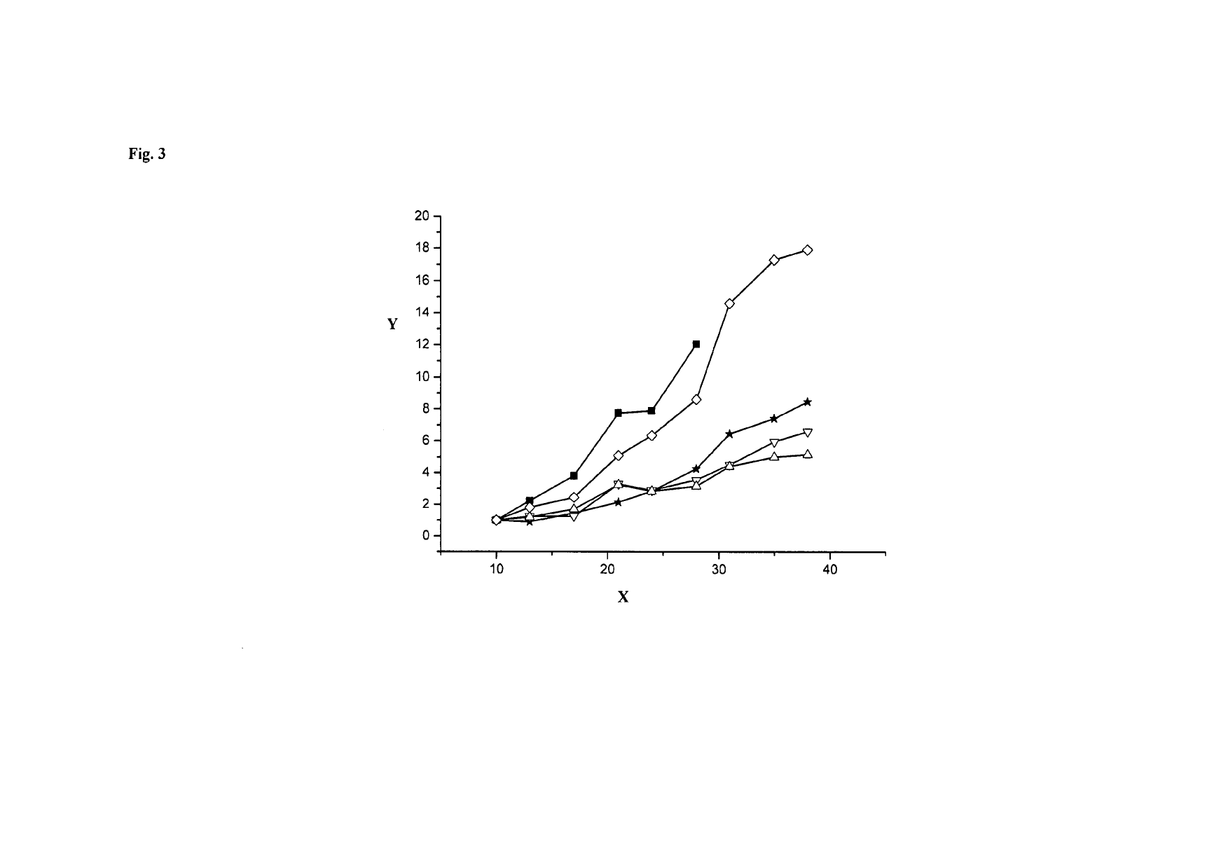

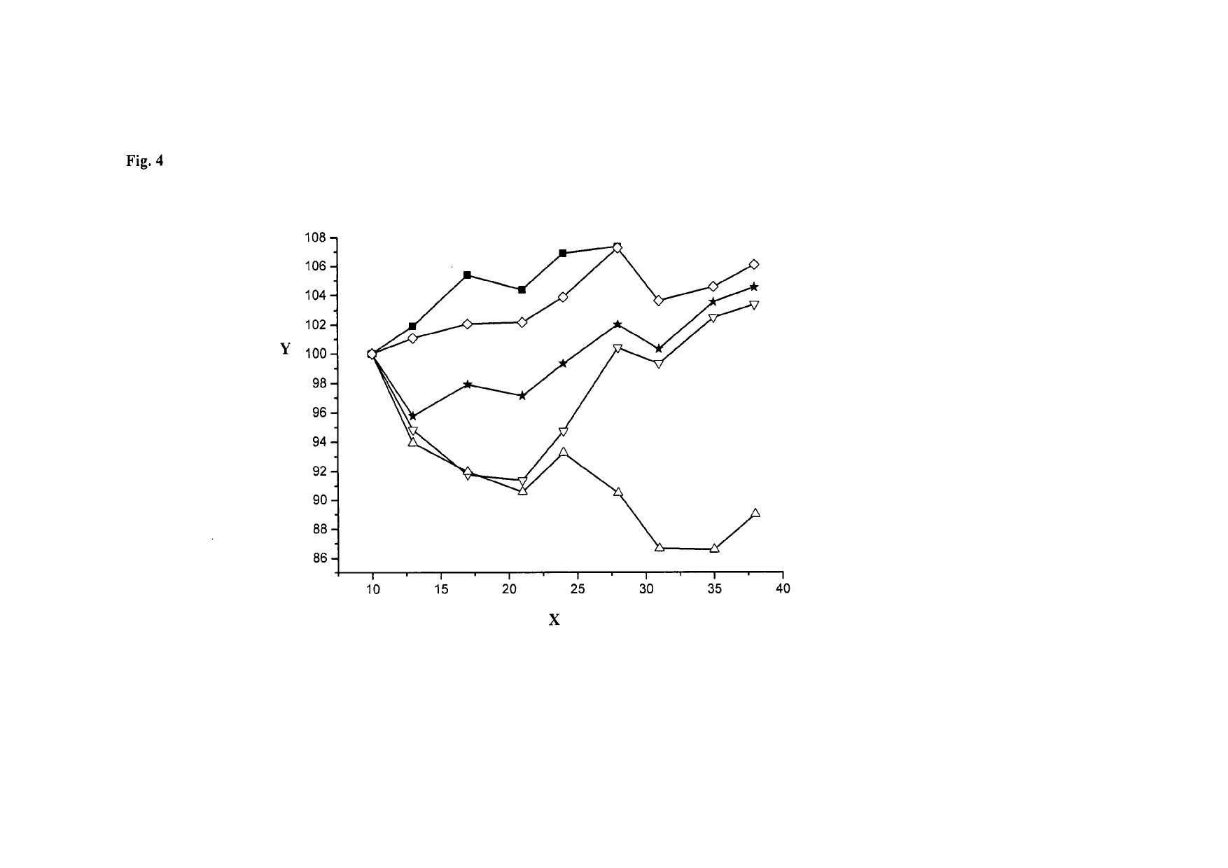

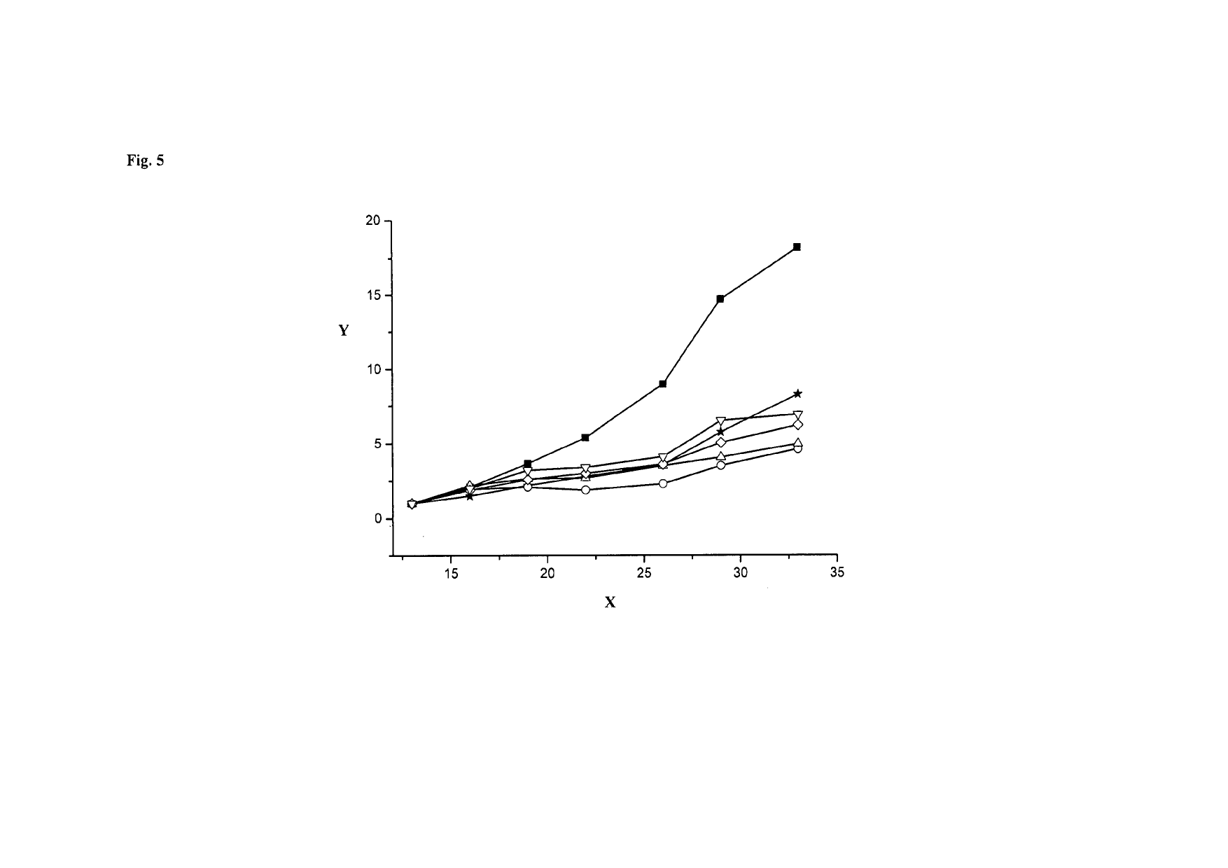

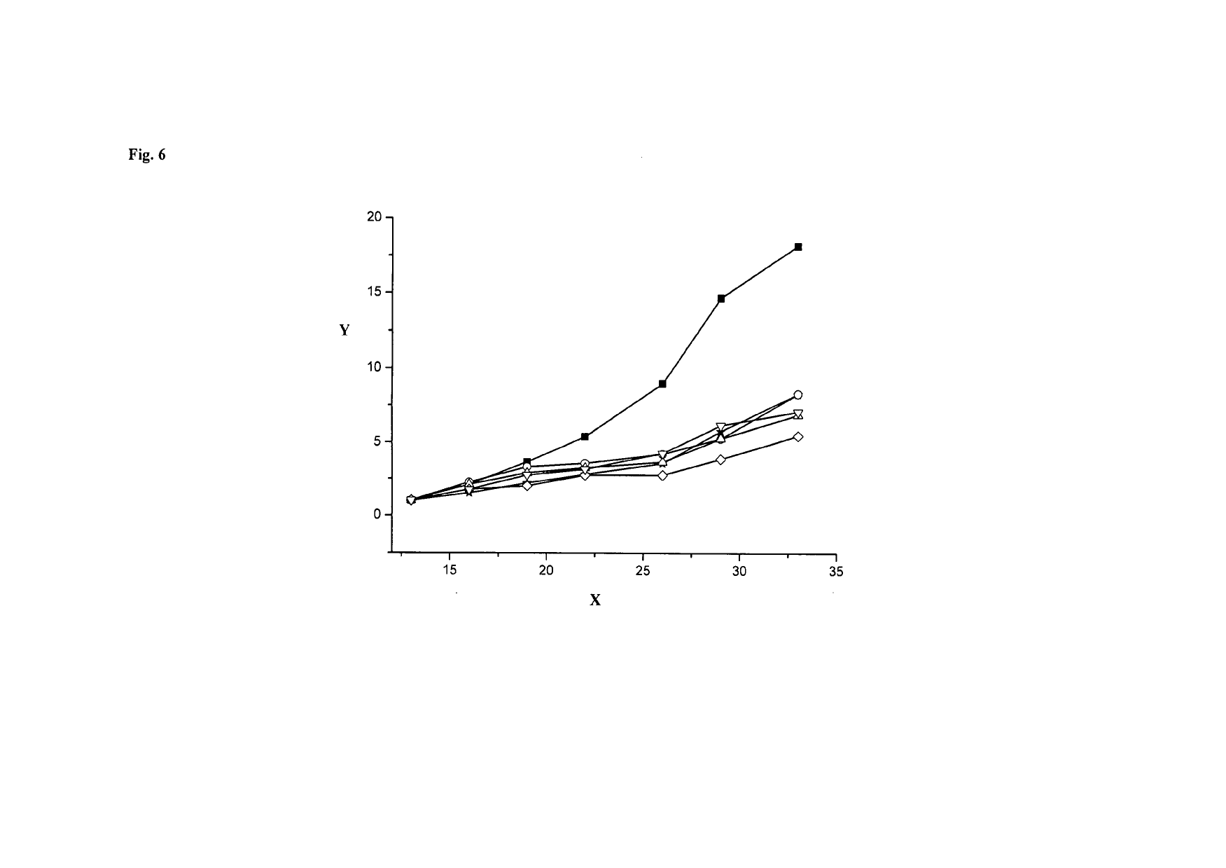

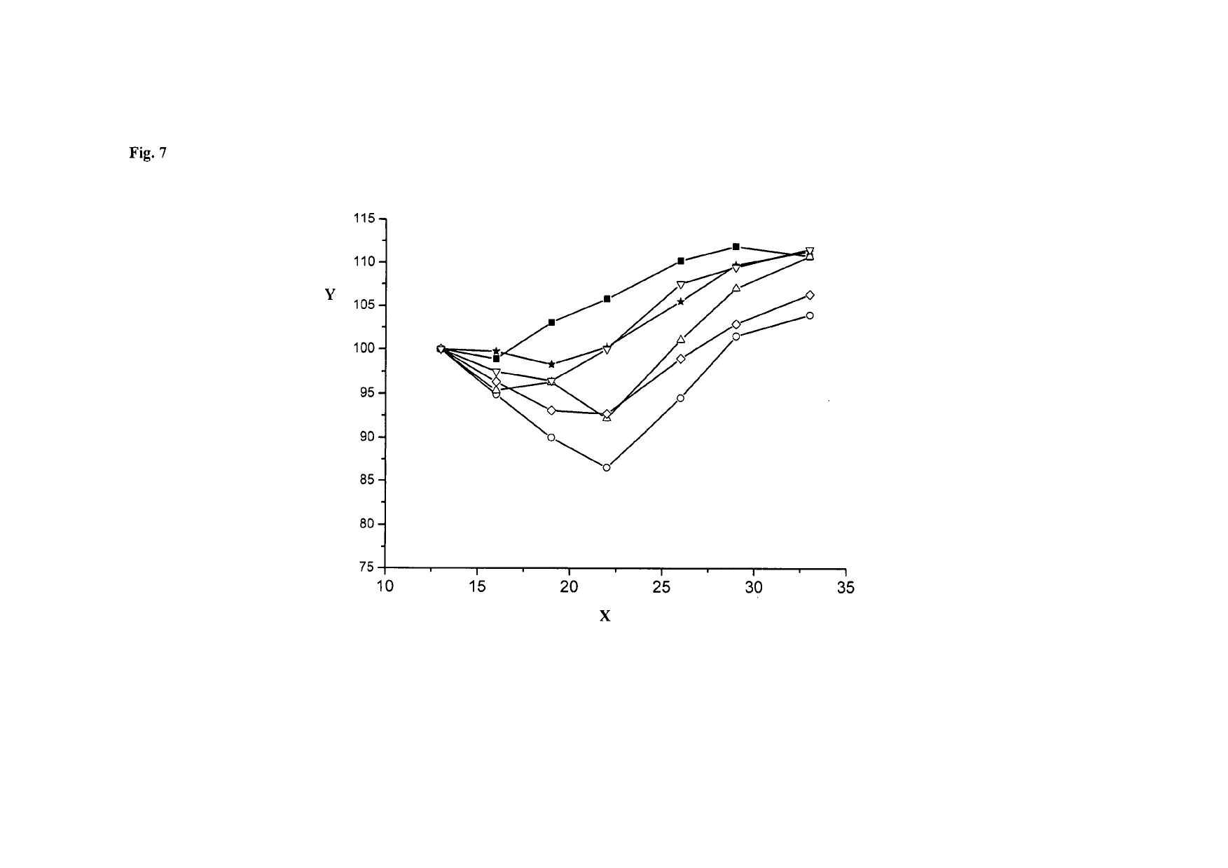

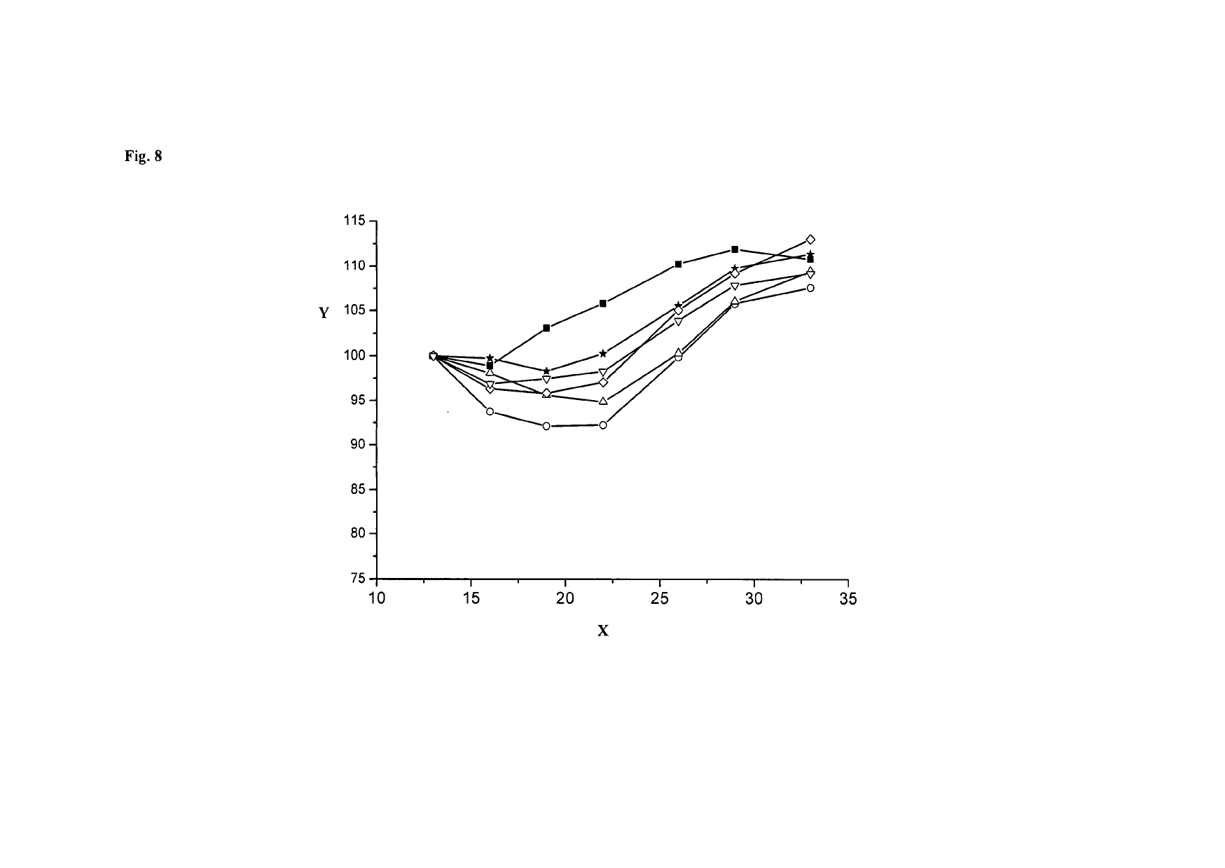

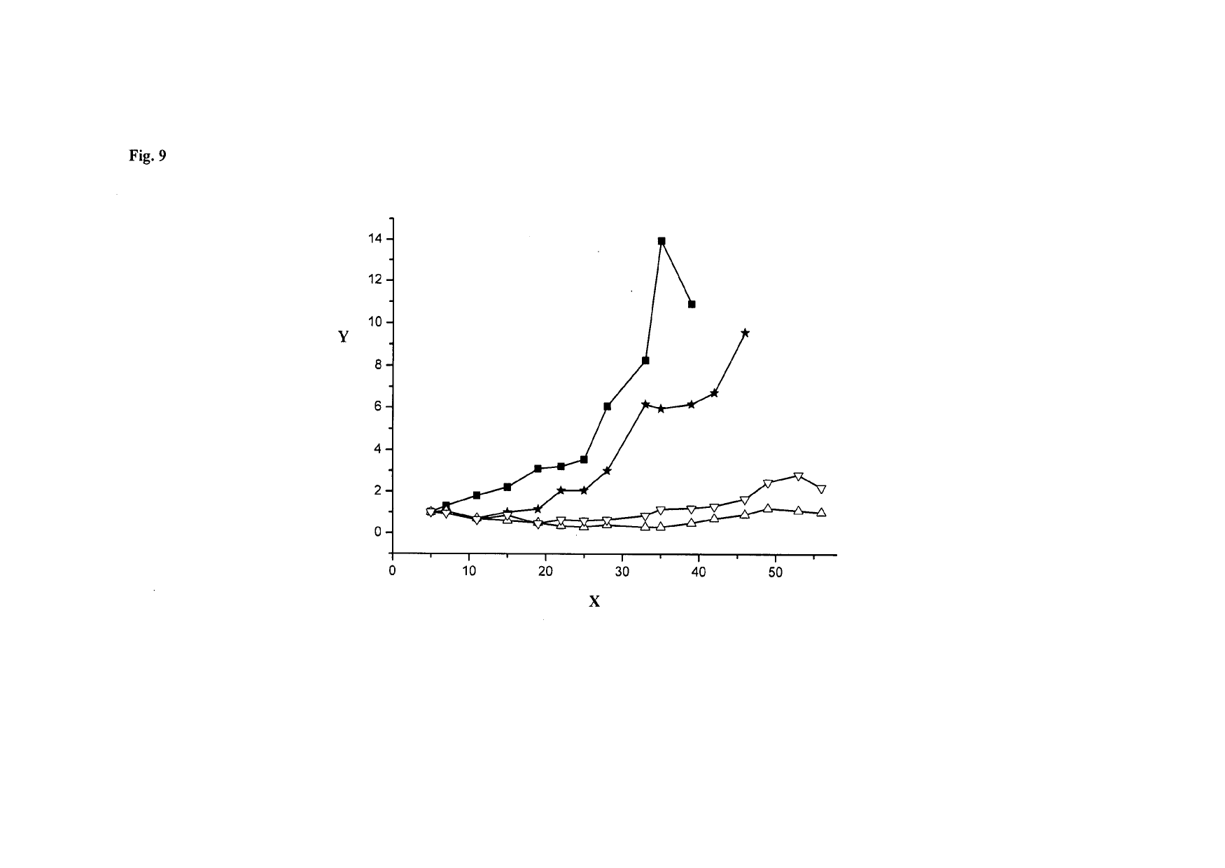

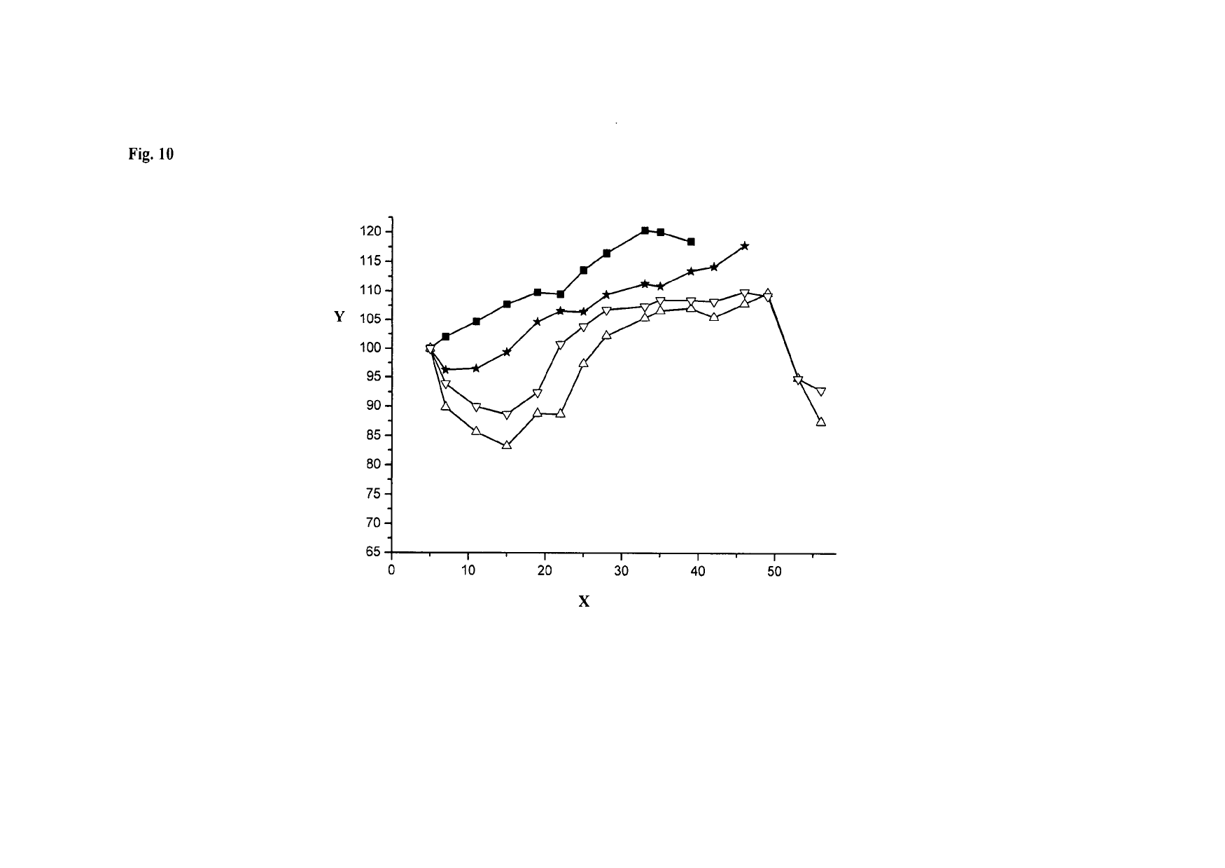

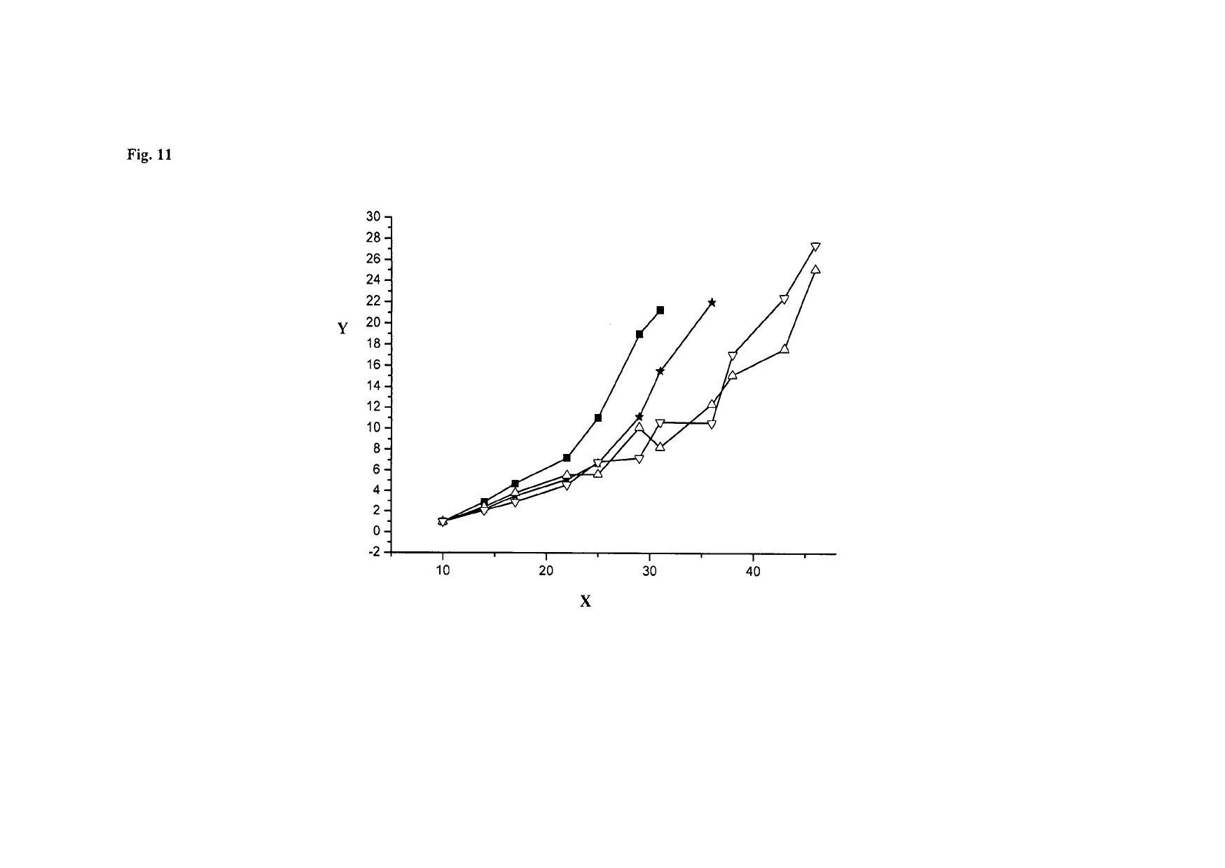

The time course of the body weight change as well as the relative tumor volume for the tested compounds and the reference compound Doxorubicin is shown in FIGS. 1 to 16.

As it can be seen from the tables 10 to 15 and the FIGS. 1 to 16, the administration of a Doxorubicin conjugates
i) allows for a more efficient inhibition of tumor growth and/or (see e.g. FIG. 9, CDx6, ovarian cancer),
ii) is less toxic (as indicated by the body weight change) than the administration of non-conjugated Doxorubicin (see e.g. FIG. 12, CDx6, mamma carcinoma).

### 2.2.1 Epirubicin and Idarubicin Studies

### 2.2.1 Test Animals

All experiments were performed using 5-6 weeks old female NMRI^{-nu/nu} nude mice (approximate weight: 30 g after acclimatization). Mice were maintained in individual ventilated cages (IVC, max. 3 mice/cage) at constant temperature and humidity. Animal weights were taken every other day (Monday, Wednesday and Friday). Animal behavior was monitored daily. At study end, animals were sacrificed and in case the tumor volume was measured a necropsy was performed.

Experimental protocols have been approved by the Ethics Committee for Animal Experimentation. The experimental protocol is registered by the Regierungspräsidium Freiburg (G10/25).

In the following an overview over the used animals and animal conditions is given:

### Test Animals:

| | |
|---|---|
| Strain | female NMRI^{-nu/nu} |
| Source | Charles River GmbH |
| | Sandhofer Weg 7 |
| | D 97633 Sulzfeld |
| Age at delivery | 5-6 weeks |
| Body weight and range | approx. 30 g |
| (at acclimatisation) | |
| Identification | Labeling by ear mark |

### Husbandry

| | |
|---|---|
| Conditions | Optimum hygienic conditions, air-conditioned with 10-15 |
| | air changes per hour, and continually monitored |
| | environment with target ranges for temperature 22 ± 3° C. |
| | and for relative humidity 30-70%, 12 hours artificial |
| | fluorescent light/12 hours dark. |
| Accommodation | max. 3 animals per individual ventilated cage (IVC) |
| Diet | M-Zucht |
| | ssniff Spezialdiäten GmbH |
| | Ferdinand Gabriel Weg 16 |
| | D-59494 Soest |
| Water | Community tap water (autoclaved) |

### 2.2.2 Tumor Model

The human breast cancer carcinoma MT-3, were used as subcutaneous (s.c.) xenotransplantation model in female NMRI:nu/nu mice
Cell Line: MT-3
DSMZ No: ACC403
Description: human breast cancer cell line; breast adenocarcinoma originated from surgical material
Morphology: adherent
Subculture: Split confluent cultures 1:6 every 3-4 days using trypsin/EDTA, cells were seeded out at approx. 2×10⁶ cells/15 cm dish+25 ml medium
Incubation: At 37° C. with 10% CO₂
Storage: Frozen with 6% DMSO in FCS
tumorigenic according to literature: yes (Zeisig et al. 2006; Wenzel J et al. 2009)
additional information: Cells do not produce estrogen receptors (Shi D.-F. et al. 1996; Hambly R J et al. 1997)

Monolayers of MT-3 cells were grown in DMEM 4500 with phenol red supplement with 10% FCS, 1% L-Glutamine, 100 units penicillin/ml, and 100 μg of streptomycin/ml. MT-3 cells were cultured in a humidified atmosphere of 90% air and 10% carbon dioxide at 37° C. Media were routinely changed every 3 days.

### Experimental Procedure

For experimental use 5×10⁶ to 10⁷ tumor cells/mouse from the in vitro passage were transplanted s.c. into the flank of each of 10 mice/group at day 0.

### Treatment

At palpable tumor size (30-100 mm³) treatment was started. The application volume was 0.2 ml/20 g mouse body weight. The test compounds, the vehicle controls and the reference compounds were all given intravenously (i.v.).

### 2.2.3 Therapeutic Evaluation

Tumor growth inhibition was used as therapeutic parameter. Additionally, body weight change was determined as signs for toxicity (particularly, potential hematological or gastrointestinal side effects).

### 2.2.4 Tested Epicrubicin and Idarubicin Conjugates

The tested Idarubicin conjugate CId3 and the Epirubicin conjugate CEp1 and CEp3 were obtained as described herein above and were kept in a freeze-dried form at −20° C. until the use. Before administration, the conjugates were dissolved in saline solution by vortexing in combination with centrifugation until a clear solution of the necessary concentration of the drug was obtained. The obtained solutions were prepared and injected under sterile conditions.

The reference compound was Epirubicin and Idarubicin (which are, for example, available as Epirubicin Sandoz (Sandoz) and Zavedos (Pfizer).

As a further control, saline solution was intravenously administered.

The above mentioned conjugates were tested in the MT-3 tumor model (breast cancer). In contrast to the MT-3 cell line used in the previous experiments described under item 2.1 above, the particular subtype used was, obtained from a different laboratory, as described above.

### 2.4.5 Test Results for Epicrubicin and Idarubicin Conjugates

Table 16 summarizes the results for the tested epicrubicin and idarubicin conjugates and the reference compound Epirubicin and Idarubicin in human breast carcinoma (MT-3) xenografts growing in nude mice.

The table shows, inter alia,
v) the tested compounds,
vi) the tumor volume in mice at the day the control group was sacrificed (in cm³),
vii) the lowest value of the relative tumor volume vs. the relative tumor volume of the control group (RTV T/C) together with the day, when this optimum was reached,
viii) the maximum body weight loss in mice together with the day, when this minimum was reached.

The loss of body weight is known to be an indicator of gastro-intestinal and hepatotoxicity of the tested compound.

In contrast to the MT-3 cell line used in the previous experiments, this particular subtype, obtained from a different laboratory, showed no sensibility towards anthracycline therapy as demonstrated for idarubicin and epirubicin. However, in case of CId3, an efficacy comparable to the unmodified drug was observed at a dose level of ⅙ the MTD for idarubicin, while no signs of toxicity could be detected. In case of epirubicin, the four-fold dose of drug in conjugated form (CEp1 and CEp3) could be applied resulting in a better performance at comparable toxicity level.

Thus, the administration of the tested idarubicin or epirubicin conjugates allows for an inhibition of tumor growth comparable to the unmodified drug while being less toxic (as indicated by the body weight change) than the administration of non-conjugated drug.

TABLE 10
Summary of the results for the tested doxorubicin conjugates (tumor model MT-3)

| | | | | | Tumor | T/C (%) | T/C (%) |
|---|---|---|---|---|---|---|---|
| | Mice | Treatment | Dose | | growth | Optimum | (TV) |
| Group | n | (d) | (mg/kg/inj.) | BWC (%) | RTV d 28 | (at day) | at the end |
| Saline | 8 | 7, 14 | | 0 | 31.1 +/− 14.8 | | |
| CDx1 | 8 | 7, 14 | 8 | −7 | 8.7 +/5.8 | 22.6 | 0.72 +/− 0.51 |
| | | | | (d 24) | | (24) | |
| CDx1 | 8 | 7, 21 | 20 | −10 | 7.2 +/− 3.6 | 21.7 | 0.52 +/− 0.42 |
| | | | | (d 14) | | (18) | + |
| Doxorubicin | 8 | 7, 14 | 8 | −8 | 10.8 +/− 3.7 | 32.2 | 1.16 +/− 0.35 |
| | | | | (d 24) | | (18) | |

TABLE 11
Summary of the results for the tested doxorubicin conjugates (tumor model MT-3)

| | | | | | Tumor | T/C (%) |
|---|---|---|---|---|---|---|
| | Mice | Treatment | Dose | BWC (%) | growth | Optimum |
| Group | n | (d) | (mg/kg/inj.) | (d 10/d 28) | RTV d 28 | (at day) |
| Saline | 8 | 10 | | 7 | 11.7 +/− 4.6 | |
| Doxorubicin | 8 | 10 | 8 | 2 | 4.3 +/− 3.4 | 30 |
| | | | | | | (21) |
| CDx1 | 8 | 10 | 20 | 0 | 3.3 +/1.13 | 26.6 |
| | | | | | | (17) |
| CDx1* | 8 | 10, 17 | 20 | −10 | 3.3 +/− 1.37 | 24.5 |
| | | | | | | (10) |
| CDx15 | 8 | 10 | 8 | 7 | 9.5 +/− 5.1 | 55.3 |
| | | | | | | (21) |

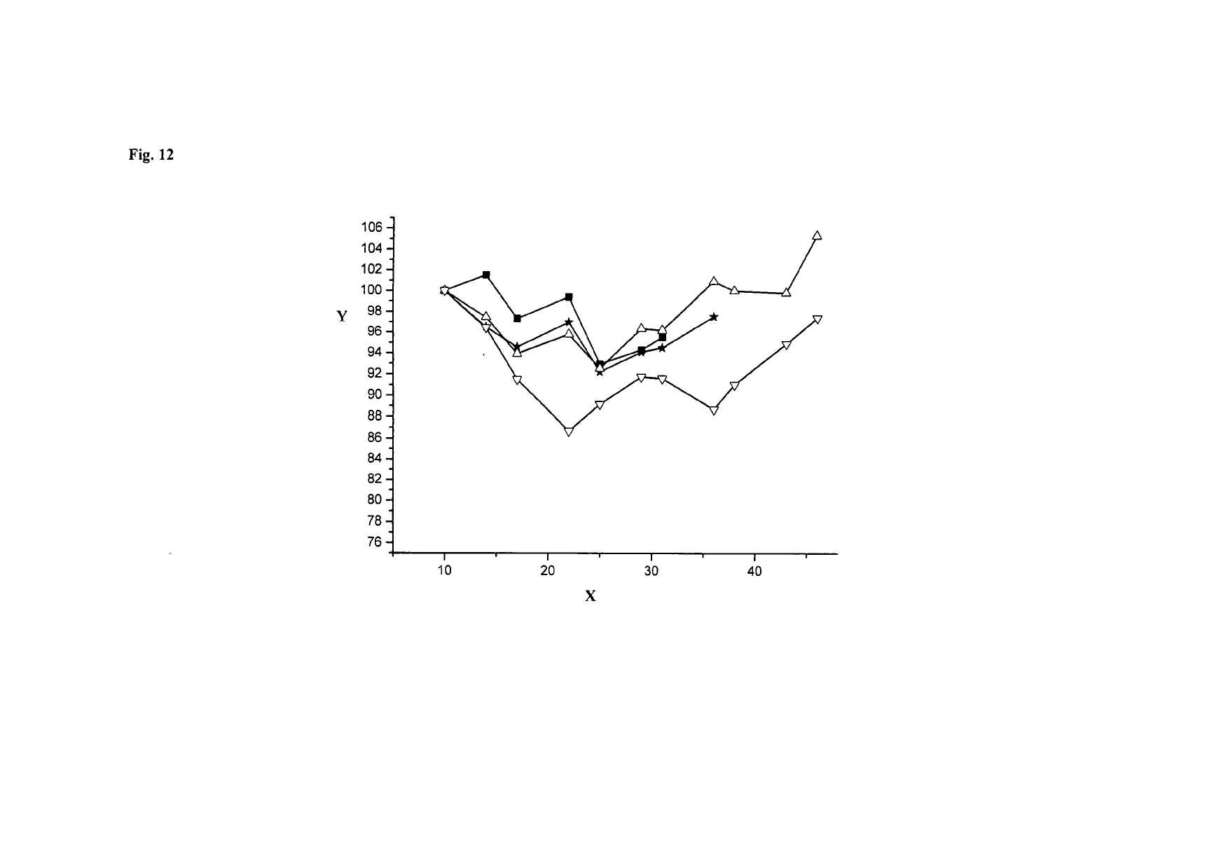

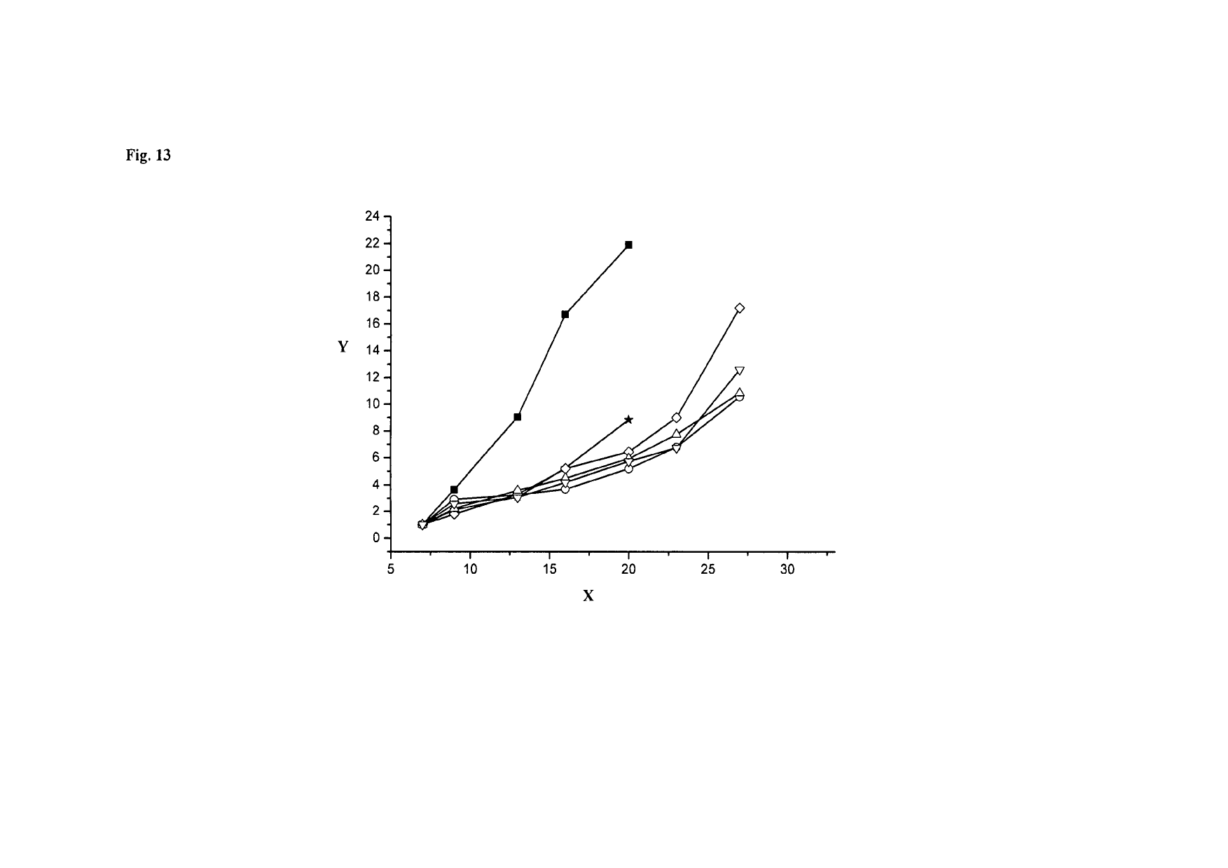

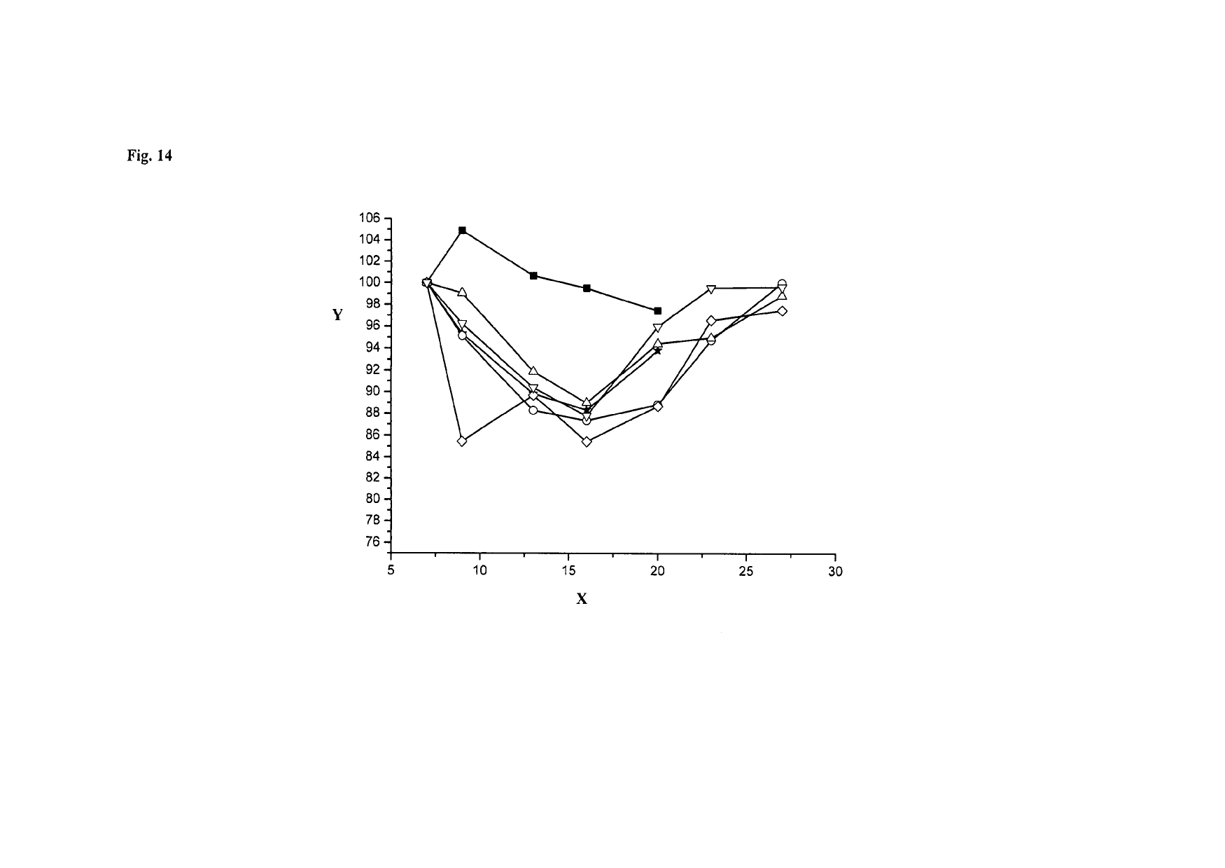

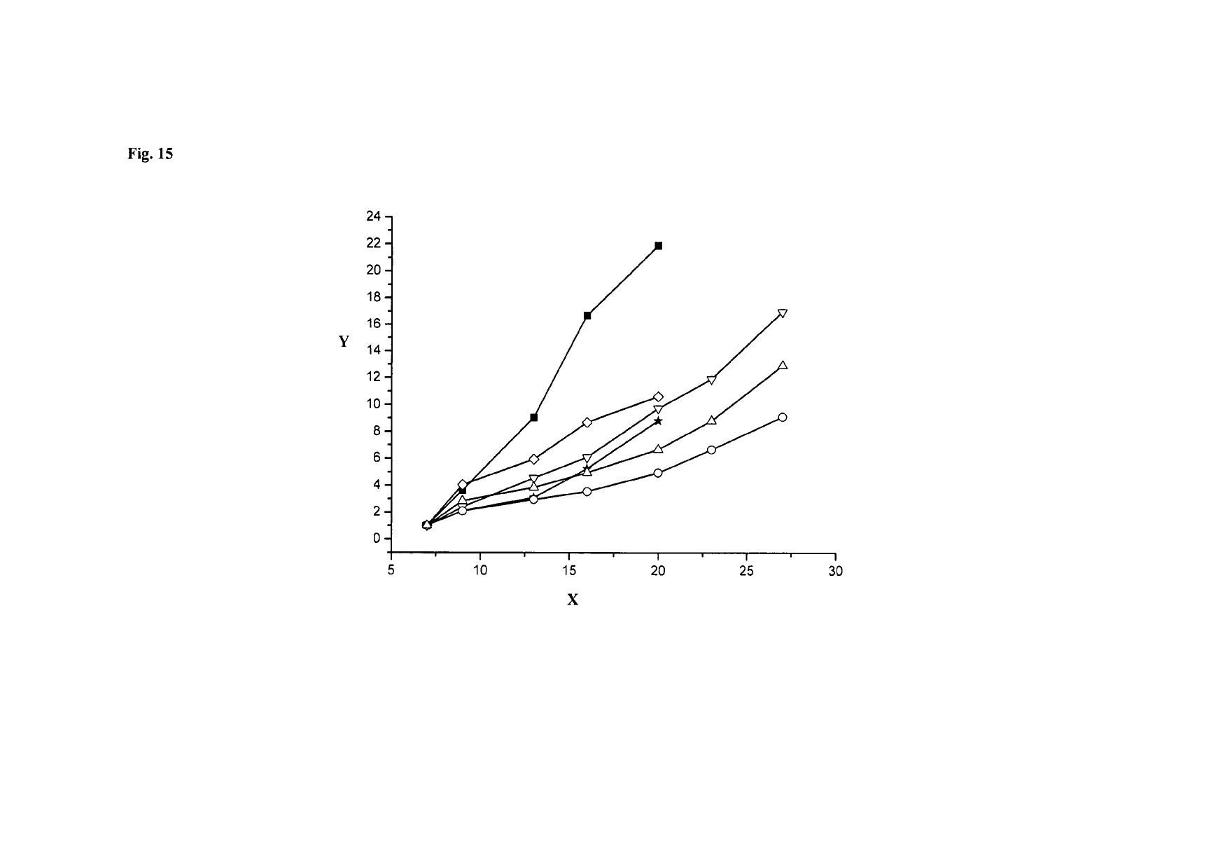

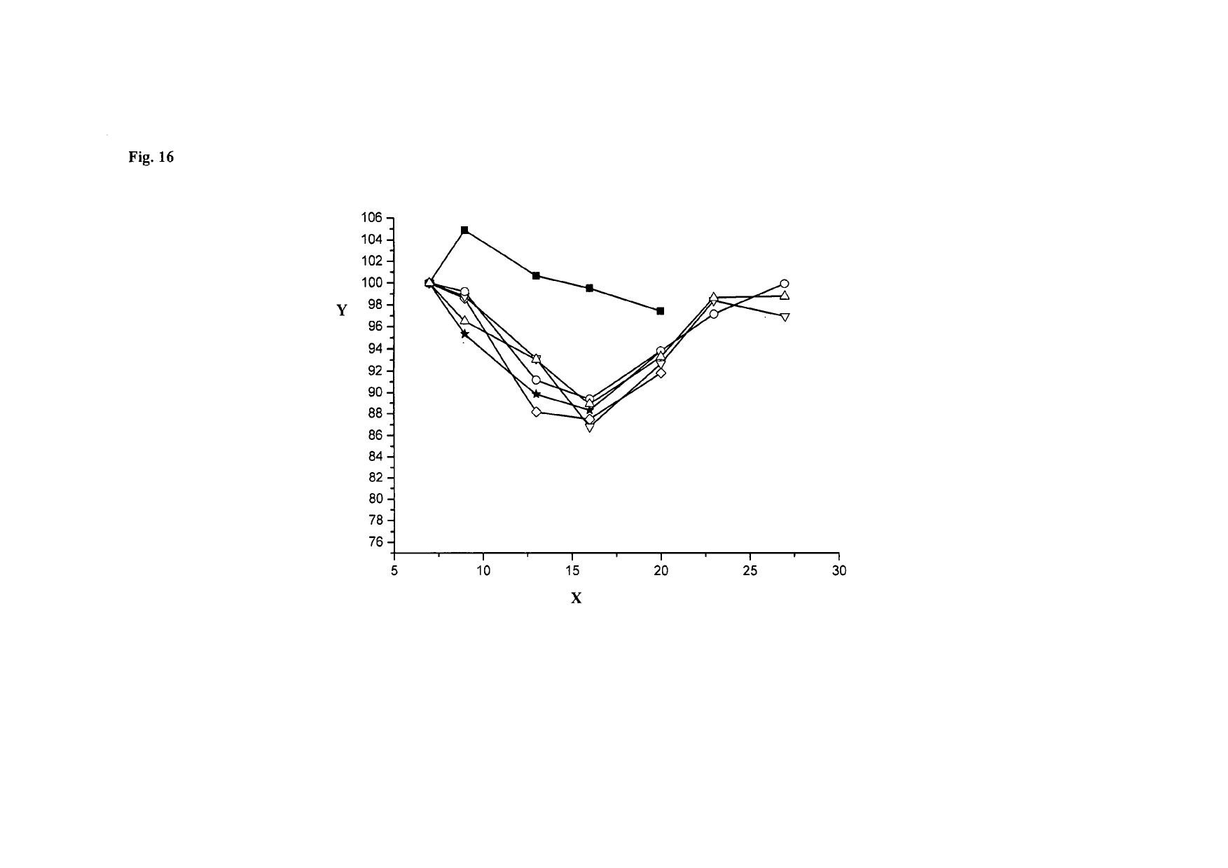

Group treated with double dosing shown as CDx1* in FIG. 3 and 4.

TABLE 12
Summary of the results for the tested doxorubicin conjugates (tumor model MT-3)

| | | | | Toxic | BWC | Tumor | RTV T/C (%) |
|---|---|---|---|---|---|---|---|
| | Mice | Treatment | Dose | deaths | [%] | growth | Optimum |
| Group | n | (d) | (mg/kg/inj.) | (at day) | (at day) | RTV d 33 | (at day) |
| Saline | 8 | 7 | | | | 14.30 +/− 5.90 | |
| Doxorubicin | 8 | 7 | 8 | | −2 | 5.59 +/− 0.78* | 39.2 |
| | | | | | (19) | | (29) |
| CDx6 | 8 | 7 | 20 | | −8 | 3.96 +/1.53* | 27.3 |
| | | | | | (22) | | (33) |
| CDc8 | 8 | 7 | 20 | | −8 | 5.59 +/− 2.22* | 35.8 |
| | | | | | (19-22) | | (29) |
| CDx9 | 8 | 7 | 20 | | −5 | 5.50 +/− 3.39* | 35.8 |
| | | | | | (22) | | (29) |
| CDx10 | 8 | 7 | 20 | 1 | −13 | 3.66 +/− 1.53* | 23.4 |
| | | | | (26) | (22) | | (29) |
| CDx11 | 8 | 7 | 20 | | −4 | 6.39 +/2.57* | 38.0 |
| | | | | | (19) | | (33) |
| CDx12 | 8 | 7 | 20 | | −4 | 4.00 +/− 1.57* | 26.3 |
| | | | | | (16-19) | | (29) |
| CDx13 | 8 | 7 | 20 | | −3 | 5.24 +/− 2.37* | 38.8 |
| | | | | | (16-19) | | (33) |
| CDx14 | 8 | 7 | 20 | | −7 | 4.71 +/1.06* | 34.2 |
| | | | | | (19-22) | | (33) |

*significantly different to saline

TABLE 13
Summary of the results for the tested doxorubicin conjugates (tumor model ovcar-3)

| | | | | Toxic | BWC | Tumor | RTV T/C (%) |
|---|---|---|---|---|---|---|---|
| | Mice | Treatment | Dose | deaths | [%] | growth | Optimum |
| Group | n | (d) | (mg/kg/inj.) | (at day) | (at day) | RTV d 39 | (at day) |
| Saline | 7 | 5 | | 0 | | 12.7 +/− 5.4 | |
| Doxorubicin | 7 | 5 | 8 | 0 | −4 | 9.86 +/− 7.98 | 37.1 |
| | | | | | (7) | | (19) |
| CDx6 | 7 | 5 | 20 | 1 | −17 | 0.44 +/− 0.26*+ | 2.2 |
| | | | | (22) | (15) | | (35) |
| CDx8 | 7 | 5 | 20 | 0 | −11 | 2.41 +/− 3.17*# | 8.2 |
| | | | | | (15) | | (35) |

TABLE 14
Summary of the results for the tested doxorubicin conjugates (tumor model MT3-ADR)

| | | | | Toxic | BWC | Tumor | RTV T/C (%) |
|---|---|---|---|---|---|---|---|
| | Mice | Treatment | Dose | deaths | [%] | growth | Optimum |
| Group | n | (d) | (mg/kg/inj.) | (at day) | (at day) | RTV d 31 | (at day) |
| Saline | 7 | 10 | | 0 | −7 | 24.9 +/− 10.7 | |
| | | | | | (25) | | |
| Doxorubicin | 7 | 10 | 8 | 0 | −8 | 15.3 +/− 5.9* | 58.4 |
| | | | | | (25) | | (29) |
| CDx6 | 7 | 10 | 20 | 0 | −7 | 11.8 +/− 7.7* | 38.5 |
| | | | | | (25) | | (31) |
| CDx8 | 7 | 10 | 20 | 0 | −13 | 10.0 +/− 4.6* | 37.9 |
| | | | | | (22) | | (29) |

TABLE 15
Summary of the results for the tested doxorubicin conjugates (tumor model MT-3)

| | | | | | Tumor | T/C (%) |
|---|---|---|---|---|---|---|
| | Mice | Treatment | Dose | BWC (%) | growth | Optimum |
| Group | n | (d) | (mg/kg/inj.) | (d 7/d 20) | RTV d 20 | (at day) |
| Saline | 8 | 7 | | −3 | 20.0 +/− 9.4 | |
| Doxorubicin | 8 | 7 | 8 | −6 | 8.1 +/− 2.1* | 37.8 |
| | | | | | | (13) |
| CDx1 | 8 | 7 | 20 | −8 | 11.9 +/4.1** | 52.6 |
| | | | | | | (20) |
| CDx2 | 8 | 7 | 20 | −7 | 9.0 +/2.2* | 31.8 |
| | | | | | | (16) |
| CDx3 | 8 | 7 | 20 | −11 | 4.7 +/1.6** | 17.1 |
| | | | | | | (20) |
| CDx4 | 8 | 7 | 20 | −6 | 7.3 +/− 1.8* | 32.7 |
| | | | | | | (16) |
| CDx5 | 8 | 7 | 20 | −4 | 5.7 +/2.7* | 30.0 |
| | | | | | | (20) |
| CDx6 | 8 | 7 | 20 | −11 | 5.1 +/− 2.4* | 29.0 |
| | | | | | | (16) |
| CDx7 | 8 | 7 | 20 | −6 | 8.6 +/− 5.1* | 39.31 |
| | | | | | | (16) |
| CDx8 | 8 | 7 | 20 | −7 | 5.0 +/− 1.9** | 27.6 |
| | | | | | | (20) |

TABLE 16
Summary of the results for the tested epirubicin
and idarubicin conjugates (tumor model MT-3)

| | | | | | | Mortality | BWC % | Min. T/C % |
|---|---|---|---|---|---|---|---|---|
| Group | Treatment | Dose level | | Schedule | Route | n | (Day) | (Day) |
| 1 | Saline | 10 | ml/kg | once on day 10 | i.v. | 0/8 | −1.5 (16) | — |
| 2 | Idarubicin | 3 | mg/kg | once on day 10 | i.v. | 0/8 | −5.1 (16) | 51.4 (19) |
| 3 | CId3 | 0.5 | mg/kg | once on day 10 | i.v. | 0/8 | 0 (16) | 57.5 (19) |
| 4 | Epirubicin | 6 | mg/kg | once on day 10 | i.v. | 0/8 | −8.5 (16) | 77.3 (16) |
| 5 | CEp1 | 24 | mg/kg | once on day 10 | i.v. | 0/8 | −10.2 (19) | 62.8 (19) |
| 6 | CEp3 | 24 | mg/kg | once on day 10 | i.v. | 0/8 | −8.0 (16) | 48.4 (19) |

1-35. (canceled)

36. A hydroxyalkyl starch (HAS) conjugate comprising a hydroxyalkyl starch derivative and a cytotoxic agent, said conjugate having a structure according to the following formula
HAS′(-M)ₙ
wherein
M is a residue of a cytotoxic agent, the cytotoxic agent comprising a carbonyl group, HAS′ is a residue of the hydroxyalkyl starch derivative comprising at least one functional group X,
n is greater than or equal to 1,
and wherein the cytotoxic agent is linked via the carbonyl function present in the cytotoxic agent to the functional group X comprised in the hydroxyalkyl starch derivative, wherein the linkage via the carbonyl function is a cleavable linkage, which is capable of being cleaved in vivo so as to release the cytotoxic agent.

37. The conjugate according to claim 36, wherein the cytotoxic agent is an anthracycline.

38. The conjugate according to claim 36, wherein the at least one functional group X comprised in HAS′ has the structure
-G′-NH—N═,
and wherein G′ is selected from the group consisting of —Y^{G}—C(=G)-, —SO₂—, aryl, and heteroaryl,
wherein G is O or S,
and wherein Y^{G} is —O—, —NH— or —NH—NH—.

39. The conjugate according to claim 38, wherein X has the structure —NH—NH—C(=G)-NH—N═, wherein G is O or S.

40. The conjugate according to claim 36, wherein the hydroxyalkyl starch derivative comprises at least one structural unit according to the following formula (I) 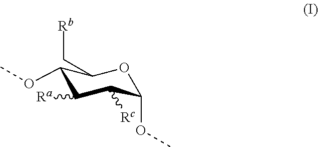
wherein R^{a}, R^{b} and R^{c} are, independently of each other, selected from the group consisting of —O-HAS″, —[O—(CR^{w}R^{x})—(CR^{y}R^{x})]ₓ—OH, and —[O—(CR^{w}R^{x})—(CR^{y}R^{z})]_{y}—[F¹]ₚ-[L¹]_{q}—[F²]ᵣ-[L²]ᵥ-X—,
and wherein R^{w}, R^{x}, R^{y} and R^{z} are independently of each other selected from the group consisting of hydrogen and alkyl,
y is an integer in the range of from 0 to 20,
x is an integer in the range of from 0 to 20,
and wherein at least one of R^{a}, R^{b} and R^{c} is —[O—(CR^{w}R^{x})—(CR^{y}R^{z})]_{y}—[F¹]ₚ-[L¹]_{q}—[F²]ᵣ[L²]ᵥ-X—,
and wherein X has the structure -G′—NH—N═, and wherein G′ is selected from the group consisting of —Y^{G}—C(=G)-, —SO₂—, aryl, and heteroaryl, wherein G is O or S, and wherein Y^{G} is —O—, —NH— or —NH—NH—,
F¹ is selected from the group consisting of —O—, —S—, —NR^{Y7} and —O—(C═Y⁶)—,
wherein Y⁶ is selected from the group consisting of NR^{Y6}, O and S, and wherein R^{Y6} is H or alkyl, and wherein R^{Y7} is H or alkyl,
p is 0 or 1,
and wherein L¹ is a linking moiety,
q is 0 or 1, with the proviso that in case p is 0, q is 0,
F² is a functional group selected from the group consisting of —S—, —NH— and —NH—NH—
T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or —NH—NH—,
r is 0 or 1,
L² is a linking moiety,
v is 0 or 1,
and wherein HAS″ is a remainder of HAS′.

41. The conjugate according to claim 40, wherein
p is 1 and wherein F¹ has the structure —O—C(═O)—, and wherein q is 0, and wherein
(i) r and v are 0 and X has the structure 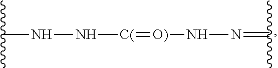
or
(ii) r and v are both 1, and wherein X has the structure -G′—NH—N═, and wherein G′ is selected from the group consisting of —Y^{G}—C(=G)-, —SO₂—, aryl, and heteroaryl, wherein G is O or S, and wherein Y^{G} is —O— or —NH—, and wherein F² has the structure —NH—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH—,
or
wherein p is 1 and q is 1 and wherein F¹ is —O— and wherein L¹ has a structure selected from the group consisting of —CH₂—CHOH—CH₂—, —CH₂—CH(CH₂OH)—, —CH₂—CHOH—CH₂—O—CH₂—CHOH—CH₂—, —CH₂—CH₂—CHOH—CH₂—, —CH₂—CH₂—CH₂—CHOH—CH₂—, and wherein
(i) r and v are 0, and X has the structure 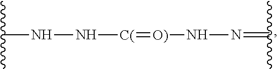
or
(ii) r and v are both 1, and wherein X has the structure -G′—NH—N═, and wherein G′ is selected from the group consisting of —Y^{G}—C(=G)-, —SO₂—, aryl, and heteroaryl, wherein G is O or S, and wherein Y^{G} is —O— or —NH—, and wherein F² has the structure —NH—NH-T′-, wherein T′ is selected from the group consisting of —C(=T)-Y^{T}—, —SO₂—, aryl, and heteroaryl, and wherein T is O or S, and wherein Y^{T} is —CH₂—, —O— or —NH—,
or
wherein p and q are 0,
(i) r and v are 0, and X has the structure 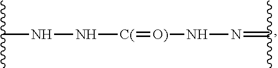
or
(ii) r and v are both 1, and wherein X has the structure -G′—NH—N═, and wherein G′ is selected from the group consisting of —Y^{G}—C(=G)-, —SO₂—, aryl, and heteroaryl, wherein G is O or S, and wherein Y^{G} is —O— or —NH—, and wherein F² has the structure —NH—NH-T′-, wherein T′ is selected from the group consisting of —C(=T)-Y^{T}—, —SO₂—, aryl, and heteroaryl, and wherein T is O or S, and wherein Y^{T} is —CH₂—, —O— or —NH—.

42. The conjugate according to claim 36, wherein the hydroxyalkyl starch derivative comprises at least one structural unit according to the following formula (I) 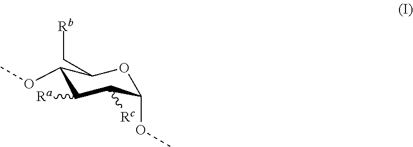
wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}—[F¹]ₚ-[L¹]_{q}—[F²]ᵣ-[L²]ᵥ-X—,
and wherein R^{w}, R^{x}, R^{y} and R^{z} are, independently of each other, selected from the group consisting of hydrogen and alkyl,
y is an integer in the range of from 0 to 20,
x is an integer in the range of from 0 to 20,
and wherein at least one of R^{a}, R^{b} and R^{c} is —[O—(CR^{w}R^{x})—(CR^{y}R_{z})]_{y}—[F¹]ₚ-[L¹]_{q}—[F²]ᵣ-[L²]ᵥ-X—,
and wherein X has the structure -G′—NH—N═, and wherein G′ is selected from the group consisting of —Y^{G}—C(=G)-, —SO₂—, aryl, and heteroaryl, wherein G is O or S,
and wherein Y^{G} is —O—, —CH₂—, —NH— or —NH—NH—,
p is 1, and F¹ is —O—,
q is 1 and L¹ is a linking moiety selected from the group consisting of —CH₂—CHOH—CH₂—, —CH₂—CH(CH₂OH)—, —CH₂—CHOH—CH₂—O—CH₂—CHOH—CH₂—, —CH₂—CH₂—CHOH—CH₂—, —CH₂—CH₂—CH₂—CHOH—CH₂—,
r is 1, and F² is a functional group having a structure selected from the group consisting of —S—, —NH— and —NH—NH-T′-, wherein T′ is selected from the group consisting of
—C(=T)-Y^{T}-, —SO₂—, aryl, and heteroaryl, and wherein T is O or S, and wherein Y^{T} is —CH₂—, —O—, —NH— or —NH—NH—,
and v is 1, and L² is selected from the group consisting of alkyl, alkenyl, alkylaryl, arylalkyl, aryl, heteroaryl, alkylheteroaryl and heteroarylalkyl,
and wherein HAS″ is a remainder of HAS′.

43. The conjugate according to claim 36, wherein the hydroxyalkyl starch derivative comprises at least one structural unit according to the following formula (I) 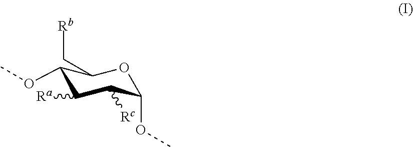
wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}—[F¹]ₚ-[L¹]_{q}—[F²]ᵣ-[L²]ᵥ-X—,
and wherein R^{w}, R^{x}, R^{y} and R^{z} are, independently of each other, selected from the group consisting of hydrogen and alkyl,
y is an integer in the range of from 0 to 20,
x is an integer in the range of from 0 to 20,
and wherein at least one of R^{a}, R^{b} and R^{c} is —[O—(CR^{w}R^{x})—(CR^{y}R^{z})]_{y}—[F¹]ₚ-[L¹]_{q}—[F²]ᵣ-[L²]ᵥ-X—,
and wherein X has the structure -G′—NH—N═, and wherein G′ is selected from the group consisting of —Y^{G}—C(=G)-, —SO₂—, aryl, and heteroaryl, wherein G is O or S,
and wherein Y^{G} is —O—, —CH₂—, —NH— or —NH—NH—,
p is 0,
q is 0,
r is 1, and F² is a functional group having the structure —NH—NH-T′-, wherein T′ is selected from the group consisting of —C(=T)-Y^{T}—, —SO₂—, aryl and heteroaryl, and wherein T is O or S, and wherein Y^{T} is —CH₂—, —O—, —NH— or —NH—NH—,
and v is 1, and L² is a linking moiety selected from the group consisting of alkyl, alkenyl, alkylaryl, arylalkyl, aryl, heteroaryl, alkylheteroaryl and heteroarylalkyl, and wherein HAS″ is a remainder of HAS.

44. The conjugate according to claim 36, wherein the conjugate has a structure according to the following formula

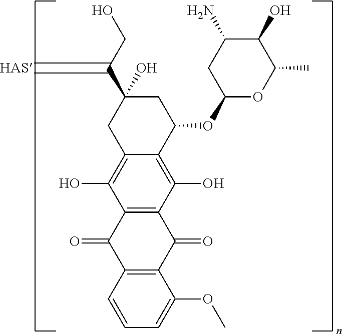

45. A method for preparing a hydroxyalkyl starch (HAS) conjugate comprising a hydroxyalkyl starch derivative and a cytotoxic agent, said conjugate having a structure according to the following formula
HAS′(-M)ₙ
wherein M is a residue of a cytotoxic agent, wherein the cytotoxic agent comprises a carbonyl group,
HAS′ is a residue of the hydroxyalkyl starch derivative comprising at least one functional group X,
n is greater than or equal to 1,
and wherein the cytotoxic agent is linked via the carbonyl function present in the cytotoxic agent to a functional group X comprised in the hydroxyalkyl starch derivative, wherein the linkage via the carbonyl function is a cleavable linkage, which is capable of being cleaved in vivo so as to release the cytotoxic agent, said method comprising
(a) providing a hydroxyalkyl starch (HAS) derivative, said HAS derivative comprising a functional group Z¹; and providing a cytotoxic agent comprising a carbonyl group;
(b) coupling the HAS derivative to the cytotoxic agent, wherein the functional group Z¹ comprised in the hydroxyalkyl starch derivative is coupled directly with the carbonyl group of the cytotoxic agent thereby forming the functional group —X—.

46. The method according to claim 45, wherein the functional group Z¹ has the structure
-G′-NH—NH₂, and wherein G′ is selected from the group consisting of —Y^{G}—C(=G)-, —SO₂—, aryl, and heteroaryl, wherein G is O or S, and wherein Y^{G} is —O—, —NH— or —NH—NH—.

47. The method according to claim 45, wherein the hydroxyalkyl starch derivative provided in step (a) comprises at least one structural unit, preferably 3 to 200 structural units, according to the following formula (I) 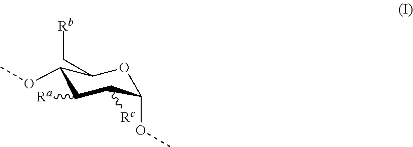
wherein R^{a}, R^{b} and R^{c} are, independently of each other, selected from the group consisting of —O-HAS″, —[O—(CR^{w}R^{x})—(CR^{y}R^{z})]ₓ—OH, and —[O—(CR^{w}R^{x})—(CR^{y}R^{z})]_{y}—[F¹]ₚ-[L¹]_{q}—[F²]ᵣ-[L²]ᵥ-Z¹,
and wherein R^{w}, R^{x}, R^{y} and R^{z}, are independently of each other, selected from the group consisting of hydrogen and alkyl,
y is an integer in the range of from 0 to 20,
x is an integer in the range of from 0 to 20,
and wherein at least one of R^{a}, R^{b} and R^{c} is —[O—(CR^{w}R^{x})—(CR^{y}R^{z})]_{y}—[F¹]ₚ-[L¹]_{q}—[F²]ᵣ-[L²]ᵥ-Z¹,
wherein F¹ is selected from the group consisting of —O—, —S—, —NR^{Y7}— and —O—(C═Y⁶)—, wherein Y⁶ is selected from the group consisting of NR^{Y6}, O and S, more preferably Y⁶ is O, and wherein R^{Y6} is H or alkyl, and wherein R^{Y7} is H or alkyl,
p is 0 or 1,
and wherein L¹ is a linking moiety selected from the group consisting of alkyl, alkenyl, alkylaryl, arylalkyl, aryl, heteroaryl, alkylheteroaryl and heteroarylalkyl,
q is 0 or 1, with the proviso that in case p is 0, q is 0,
F² is a functional group selected from the group consisting of —S—, —NH— and —NH—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or
—NH—NH—,
r is 0 or 1,
L² is a linking moiety selected from the group consisting of alkyl, alkenyl, alkylaryl, arylalkyl, aryl, heteroaryl, alkylheteroaryl and heteroarylalkyl,
v is 0 or 1,
and wherein HAS″ is a remainder of HAS′,
and wherein step (a) comprises
(a1) providing a hydroxyalkyl starch comprising the structural unit according to the following formula (II) 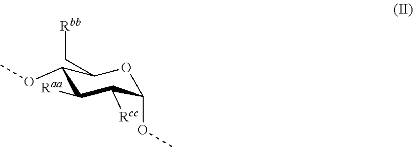
wherein R^{aa}, R^{bb} and R^{cc} are, independently of each other, selected from the group consisting of —O-HAS″ and —[O—(CR^{w}R^{x})—(CR^{y}R^{z})]ₓ—OH,
and wherein R^{w}, R^{x}, R^{y} and R^{z} are, independently of each other, selected from the group consisting of hydrogen and alkyl, and wherein
x is an integer in the range of from 0 to 20,
(a2) introducing at least one functional group Z¹ into HAS by
(i) coupling the hydroxyalkyl starch via at least one hydroxyl group comprised in HAS to at least one suitable linker comprising the functional group Z¹ or a precursor of the functional group Z¹, or
(ii) displacing at least one hydroxyl group comprised in HAS in a substitution reaction with a suitable linker comprising the functional group Z¹ or a precursor thereof.

48. The method according to claim 47, wherein step (a2)(i) comprises
(aa) activating at least one hydroxyl group comprised in the hydroxyalkyl starch with a reactive carbonyl compound having the structure R**—(C═O)—R*, wherein R* and R** may be the same or different, and wherein R* and R** are both leaving groups, wherein upon activation a hydroxyalkyl starch derivative comprising at least one structural unit according to the following formula (Ib) 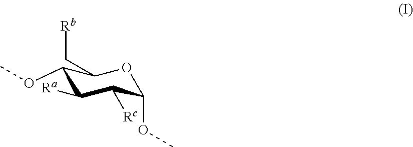
is formed, in which R^{a}, R^{b} and R^{c} are independently of each other selected from the group consisting of —O-HAS″, —[O—CH₂—CH₂]ₛ—OH, and —[O—CH₂—CH₂]ₜ—O—C(═O)—R*,
wherein s is in the range of from 0 to 4,
and wherein t is in the range of from 0 to 4,
wherein at least one of R^{a}, R^{b} and R^{c} comprises the group —[O—CH₂—CH₂]ₜ—O—C(═O)—R*, and
(bb) reacting the activated hydroxyalkyl starch according to step (aa) with the suitable linker comprising the functional group Z¹ or a precursor of the functional group Z¹, preferably wherein the activated hydroxyalkyl starch derivative is reacted with a linker having the structure Z²-[L²]ᵥ-Z¹, wherein
v is 1, and Z² has a structure according to the formula H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or —NH—NH—,
and wherein upon reaction of Z²-[L²]ᵥ-Z¹ with the group —O—C(═O)R^{*} comprised in the hydroxyalkyl starch derivative, the structural unit —[F¹]ₚ-[L¹]_{q}—[F²]ᵣ-[L²]ᵥ-Z¹ is formed, with q being 1, and with F¹ being —O—C(═O)— and with F² being —NH—NH-T′-, or wherein
v is 0, Z² is H, and Z¹ has the structure 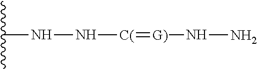
and wherein upon reaction of Z²-[L²]ᵥ-Z¹ with the group —O—C(═O)R* comprised in the hydroxyalkyl starch derivative, the structural unit —[F¹]ₚ-[L¹]_{q}—[F²]ᵣ-[L²]ᵥ-Z¹ is formed, with q, r and v being 0.

49. The method according to claim 47, wherein (a2)(i) comprises
(I) coupling the hydroxyalkyl starch via at least one hydroxyl group comprised in the hydroxyalkyl starch with a first linker comprising a functional group K², K² being capable of being reacted with a hydroxyl group of the hydroxyalkyl starch, thereby forming a covalent linkage, the first linker further comprising a functional group W, with W being a precursor of the functional group Z¹, and wherein the functional group W is an epoxide or a group which is transformed in a further step to give an epoxide,
preferably wherein the first linker has a structure according to the formula K²-L^{W}-W, wherein
K² is a functional group capable of being reacted with a hydroxyl group of the hydroxyalkyl starch,
L^{W} is a linking moiety,
wherein upon reaction of the hydroxyalkyl starch with the first linker, a hydroxyalkyl starch derivative is formed comprising at least one structural unit according to the following formula (I) 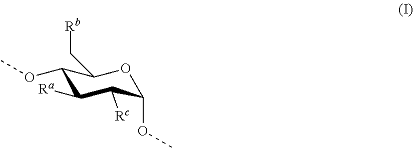
wherein R^{a}, R^{b} and R^{c} are independently of each other selected from the group consisting of —O-HAS″, —[O—CH₂—CH₂]ₛ—OH and —[O—CH₂—CH₂]ₜ[F¹]ₚ-L^{W}-W,
and wherein at least one of R^{a}, R^{b} and R^{c} is —[O—CH₂—CH₂]—[F¹]ₚ-L^{W}-W,
s is in the range of from 0 to 4,
and t is in the range of from 0 to 4,
p is 1,
and wherein F¹ is a functional group which is formed upon reaction of K² with a hydroxyl group of the hydroxyalkyl starch, wherein F¹ is preferably —O—,
and wherein HAS″ is a remainder of HAS′.

50. The method according to claim 49, wherein W is an alkenyl group and the method further comprises
(II) oxidizing the alkenyl group W to give the epoxide,
and wherein in step (II) a hydroxyalkyl starch derivative comprising at least one structural unit according to the following formula (I) 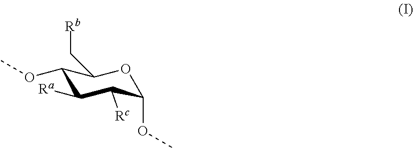
is formed, wherein R^{a}, R^{b} and R^{c}, are independently of each other, selected from the group consisting of —O-HAS″, —[O—CH₂—CH₂]ₛ—OH and 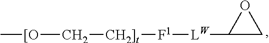
and wherein at least one of R^{a}, R^{b} and R^{c} is 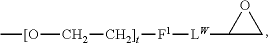
(III)reacting the epoxide with a linker having the structure Z²-[L²]ᵥ-Z¹ or Z²-[L²]ᵥ-Z¹* —PG, wherein PG is a suitable protecting group, and Z¹* being the protected form of the functional group Z¹, and wherein
v is 1, and Z² has a structure selected from the group consisting of HS—, H₂N— and H₂N—NH-T′-, wherein T′ is selected from the group consisting of —C(=T)-Y^{T}—,
—SO₂—, aryl, and heteroaryl, and wherein T is O or S, and wherein Y^{T} is —CH₂—, —O—, —NH— or —NH—NH—,
and wherein upon reaction the linker with the structural unit 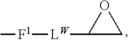
comprised in the hydroxyalkyl starch derivative, the structural unit —[F¹]ₚ-[L¹]_{q}—[F²]ᵣ-[L²]ᵥ-Z¹ is formed, with q being 1, with p being 1 and with F¹ being —O—, and with r being 1, and F² is selected from the group consisting of —S—, —HN— and —NH—NH-T′, or wherein
v is 0, and Z² is H and Z¹ has the structure 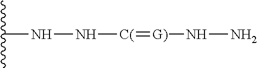
and wherein upon reaction of Z²-[L²]ᵥ-Z¹ with the structural unit 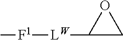
comprised in the hydroxyalkyl starch derivative, the structural unit —[F¹]ₚ-[L¹]_{q}—[F²]ᵣ-[L²]ᵥ-Z¹ is formed, with q being 1, and with r being 0.

51. The method according to claim 47, wherein in step (a2)(ii), prior to the displacement of the hydroxyl group, a group R^{L} is added to at least one hydroxyl group, thereby generating a group —O—R^{L}, wherein —O—R^{L} is a leaving group, in particular an —O-Mesyl (-OMs) or an —O-Tosyl (—O-Ts) group.

52. The method according to claim 47, wherein the suitable linker according to step (a2)(ii) has the structure Z²-[L²]ᵥ-Z¹, wherein
v is 1, and Z² has a structure according to the formula H₂N—NH-T′-, wherein T′ is selected from the group consisting of —C(=T)-Y^{T}—, —SO₂—, aryl, and heteroaryl, and wherein T is O or S, and wherein Y^{T} is —CH₂—, —O—, —NH— or —NH—NH—,
and wherein upon reaction the structural unit —[F¹]ₚ-[L¹]_{q}—[F²]ᵣ-[L²]ᵥ-Z¹ is formed, with p and q being 0, with r being 1 and with F² being selected from the group consisting of —S—, —NH— and —NH—NH-T′-, or wherein
v is 0 and Z² is H and Z¹ has the structure 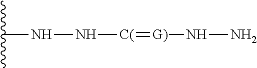
and wherein upon reaction the structural unit —[F¹]ₚ-[L¹]_{q}—[F²]ᵣ-[L²]ᵥ-Z¹ is formed, with p, q, r and v being 0.

53. The method according to claim 45, wherein the cytotoxic agent is an anthracycline.

54. A hydroxyalkyl starch conjugate obtained by a method according to claim 45.

55. A pharmaceutical composition comprising a conjugate according to claim 36 and a pharmaceutically acceptable carrier.

56. A hydroxyalkyl starch conjugate according to any of claim 36 for use as a medicament.

57. A hydroxyalkyl starch conjugate according to claim 36 for use in treating cancer.

58. Use of a hydroxyalkyl starch conjugate according to claim 36 for the manufacture of a medicament for the treatment of cancer.
